(19) United States
(12) Patent Application Publication
(10) Pub. No.: US 2024/0295486 A1
(43) Pub. Date: Sep. 5, 2024

(54) SYSTEMS AND METHODS FOR ANALYZING CYTOMETRY DATA

(71) Applicant: BostonGene Corporation, Waltham, MA (US)

(72) Inventors: Aleksandr Zaitsev, Yerevan (AM); Dmitrii Fastovets, Dilijan (AM); Roman Kruglikov, Yerevan (AM); Anatoly Bobe, Dilijan (AM); Anna Kamysheva, Yerevan (AM); Ilya Krauz, Yerevan (AM); Anastasiia Kilina, Yerevan (AM); Pavel Ovcharov, Yerevan (AM); Alexander Ryabyhk, Yerevan (AM)

(21) Appl. No.: 18/592,812

(22) Filed: Mar. 1, 2024

Related U.S. Application Data
(60) Provisional application No. 63/487,851, filed on Mar. 1, 2023.

(51) Int. Cl. G01N 15/1429; G01N 15/10; G01N 15/14

(52) U.S. Cl. CPC G01N 15/1429; G01N 15/1456; G01N 2015/1006; G01N 2015/1461

(57) ABSTRACT

Techniques for identifying types of cells present in a biological sample using flow cytometry performed using a panel of markers and multiple machine learning models. The techniques include: obtaining flow cytometry data for the biological sample, the biological sample previously-obtained from a subject and comprising a plurality of cells, the flow cytometry data including flow cytometry measurements obtained during respective flow cytometry events, the flow cytometry events including a subset of events corresponding to cells in the biological sample being measured by the flow cytometry platform; and identifying types of cells of the plurality of cells using the multiple machine learning models to obtain a respective plurality of cell types.

### CROSS-REFERENCE TO RELATED APPLICATIONS

This application claims the benefit under 35 U.S.C. § 119(e) of the filing date of U.S. Provisional Application No. 63/487,851, filed Mar. 1, 2023, entitled “SYSTEMS AND METHODS FOR ANALYZING CYTOMETRY DATA”, the entire contents of which are incorporated by reference herein.

### BACKGROUND

Cytometry is a laboratory technique used for analyzing single cells or particles in a biological sample. Cytometry is used in a variety of applications such as immunology and molecular biology. Cytometry may be used to measure characteristics of individual cells or particles. Types of cytometry include flow cytometry and mass cytometry.

Flow cytometry measures the intensity produced by fluorescent markers that are used to label cells in the biological sample. For example, a cell labelled with one or more markers may be processed by a flow cytometry platform, which measures the fluorescence intensities of the marker(s). The measured fluorescence intensities may be termed “marker values” and may be used for various applications such as cell counting, cell sorting, and/or determining various cell characteristics. Other types of cytometry (e.g., mass cytometry) may also be used for such applications.

When obtaining flow cytometry data for a biological sample, the biological sample may be partitioned into multiple sub-samples. Each sub-sample may be processed using a different set of markers, which may be termed a “panel” of markers. A panel of markers is the set of markers used to label cells in the biological sample or sub-sample. Since different markers bind to different cell types or subtypes, using different panels of markers to obtain cytometry data allows for the identification of different cell types in the biological sample.

### SUMMARY

Some embodiments provide for a method for identifying types of cells present in a biological sample using flow cytometry performed using a panel of markers and a set of machine learning models each of which corresponds to a respective marker in the panel of markers. In some embodiments, the method comprises: using at least one computer hardware processor to perform: obtaining flow cytometry data for the biological sample, the biological sample previously obtained from a subject and comprising a plurality of cells including a first cell, the flow cytometry data including flow cytometry measurements of cells in the plurality of cells that were obtained by a flow cytometry platform; and identifying cell types of at least a subset of the plurality of cells using the set of machine learning models to obtain a plurality of cell types, the set of machine learning models including a first machine learning model corresponding to a first marker in the panel of markers and a second machine learning model corresponding to a second marker in the panel of markers, the identifying comprising: obtaining, from the flow cytometry data, first flow cytometry measurements of the first cell obtained by the flow cytometry platform; processing the first flow cytometry measurements using the first machine learning model to obtain first output indicating a degree to which the first marker is expressed in the first cell; processing the first flow cytometry measurements using the second machine learning model to obtain second output indicating a degree to which the second marker is expressed in the first cell; and identifying a cell type for the first cell using the first output indicating the degree to which the first marker is expressed in the first cell and the second output indicating the degree to which the second marker is expressed in the first cell.

Some embodiments provide for at least one non-transitory computer-readable storage medium storing processor-executable instructions that, when executed by at least one computer hardware processor, cause the at least one computer hardware processor to perform a method for identifying types of cells present in a biological sample using flow cytometry performed using a panel of markers and a set of machine learning models each of which corresponds to a respective marker in the panel of markers. In some embodiments, the method comprises: obtaining flow cytometry data for the biological sample, the biological sample previously obtained from a subject and comprising a plurality of cells including a first cell, the flow cytometry data including flow cytometry measurements of cells in the plurality of cells that were obtained by a flow cytometry platform; and identifying cell types of at least a subset of the plurality of cells using the set of machine learning models to obtain a plurality of cell types, the set of machine learning models including a first machine learning model corresponding to a first marker in the panel of markers and a second machine learning model corresponding to a second marker in the panel of markers, the identifying comprising: obtaining, from the flow cytometry data, first flow cytometry measurements of the first cell obtained by the flow cytometry platform; processing the first flow cytometry measurements using the first machine learning model to obtain first output indicating a degree to which the first marker is expressed in the first cell; processing the first flow cytometry measurements using the second machine learning model to obtain second output indicating a degree to which the second marker is expressed in the first cell; and identifying a cell type for the first cell using the first output indicating the degree to which the first marker is expressed in the first cell and the second output indicating the degree to which the second marker is expressed in the first cell.

Some embodiments provide for a system comprising: at least one computer hardware processor; and at least one non-transitory computer-readable storage medium storing processor-executable instructions that, when executed by the at least one computer hardware processor, cause the at least one computer hardware process to perform a method for identifying types of cells present in a biological sample using flow cytometry performed using a panel of markers and a set of machine learning models each of which corresponds to a respective marker in the panel of markers. In some embodiments, the method comprises: obtaining flow cytometry data for the biological sample, the biological sample previously obtained from a subject and comprising a plurality of cells including a first cell, the flow cytometry data including flow cytometry measurements of cells in the plurality of cells that were obtained by a flow cytometry platform; and identifying cell types of at least a subset of the plurality of cells using the set of machine learning models to obtain a plurality of cell types, the set of machine learning models including a first machine learning model corresponding to a first marker in the panel of markers and a second machine learning model corresponding to a second marker in the panel of markers, the identifying comprising: obtaining, from the flow cytometry data, first flow cytometry measurements of the first cell obtained by the flow cytometry platform; processing the first flow cytometry measurements using the first machine learning model to obtain first output indicating a degree to which the first marker is expressed in the first cell; processing the first flow cytometry measurements using the second machine learning model to obtain second output indicating a degree to which the second marker is expressed in the first cell; and identifying a cell type for the first cell using the first output indicating the degree to which the first marker is expressed in the first cell and the second output indicating the degree to which the second marker is expressed in the first cell.

In some embodiments, the subset of the plurality of cells comprises at least 100 cells, at least 500 cells, at least 1,000 cells, at least 2,500 cells, at least 5,000 cells, at least 10,000 cells, at least 25,000 cells, at least 75,000 cells, at least 100,000 cells, at least 150,000 cells, at least 200,000 cells, or at least 500,000 cells.

In some embodiments, identifying cell types of at least the subset of the plurality of cells using the set of machine learning models further comprises, for a second cell in the plurality of cells, obtaining, from the flow cytometry data, second flow cytometry measurements of the second cell obtained by the flow cytometry platform; processing the second flow cytometry measurements using the first machine learning model to obtain a third output indicating a degree to which the first marker is expressed in the second cell; processing the second flow cytometry measurements using the second machine learning model to obtain fourth output indicating a degree to which the second marker is expressed in the second cell; and identifying a cell type for the second cell using the third output indicating the degree to which the first marker is expressed in the second cell and the fourth output indicating the degree to which the second marker is expressed in the second cell.

In some embodiments, the first output indicates whether expression of the first marker in the first cell exceeds a first threshold.

In some embodiments, the first output indicates whether the first marker is expressed in the first cell.

In some embodiments, the first machine learning model is a binary classifier.

In some embodiments, the first machine learning model comprises a decision tree classifier or a gradient-boosted decision tree classifier.

In some embodiments, the first machine learning model comprises a gradient-boosted decision tree classifier.

In some embodiments, the gradient-boosted decision tree classifier comprises an ensemble of decision tree classifiers.

In some embodiments, the gradient-boosted decision tree classifier is implemented using LightGBM, XGBoost, or CatBoost.

In some embodiments, the panel of markers comprises at least five markers, and the set of machine learning models comprises at least five machine learning models corresponding to the at least five markers.

In some embodiments, the panel of markers comprises at least ten markers, and the set of machine learning models comprises at least ten machine learning models corresponding to the at least ten markers.

In some embodiments, the set of machine learning models further comprises a third machine learning model corresponding to a third marker in the panel of markers, and identifying the types of cells of the plurality of cells using the set of machine learning models further comprises: processing the first flow cytometry measurements using the third machine learning model to obtain a third output indicating a degree to which the third marker is expressed in the first cell; and identifying the cell type for the first cell using at least the first output indicating the degree to which the first marker is expressed in the first cell, the second output indicating the degree to which the second marker is expressed in the first cell, and the third output indicating the degree to which the third marker is expressed in the first cell.

Some embodiments further comprise: processing the first flow cytometry measurements using each of the set of machine learning models to obtain multiple outputs each indicating a degree to which a respective marker in the panel of markers is expressed in the first cell, the multiple outputs including the first output and the second output; and identifying the cell type for the first cell using the multiple outputs.

In some embodiments, identifying the cell type for the first cell using the first output and the second output comprises identifying a cell type associated with the indicated degree to which the first marker is expressed in the first cell and the indicated degree to which the second marker is expressed in the first cell.

In some embodiments, identifying the cell type associated with the indicated degree to which the first marker is expressed in the first cell and the indicated degree to which the second marker is expressed in the first cell comprises identifying the cell type from the group of cell types listed in Table 1B, Table 2C, Table 7B, Table 8C, or Table 10C.

In some embodiments, the panel of markers comprises: CD45, CD66b, and CD193 CCR3, and the set of machine learning models comprises: a machine learning model trained to predict, from flow cytometry measurements of a cell, whether CD45 is expressed by the cell, the flow cytometry measurements of the cell including expression levels for a plurality of markers including CD45, CD66b, and CD193 CCR3, a machine learning model trained to predict, from the flow cytometry measurements of the cell, whether CD66b is expressed by the cell, and a machine learning model trained to predict, from the flow cytometry measurements of the cell, whether CD193 CCR3 is expressed by the cell.

In some embodiments, identifying cell types of at least the subset of the plurality of cells using the set of machine learning models further comprises, for a second cell in the plurality of cells: obtaining, from the flow cytometry data, second flow cytometry measurements of the second cell obtained by the flow cytometry platform; processing the second flow cytometry measurements using the machine learning model trained to predict whether CD45 is expressed by the cell to obtain output indicating whether CD45 is expressed in the second cell; processing the second flow cytometry measurements using the machine learning model trained to predict whether CD66b is expressed by the cell to obtain output indicating whether CD66b is expressed in the second cell; processing the second flow cytometry measurements using the machine learning model trained to predict whether CD193 CCR3 is expressed by the cell to obtain output indicating whether CD193 CCR3 is expressed in the second cell; and identifying a type for the second cell using the output indicating whether CD45 is expressed in the second cell, the output indicating whether CD66b is expressed in the second cell, and the output indicating whether CD193 CCR3 is expressed in the second cell.

In some embodiments, identifying the type for the second cell comprises: identifying eosinophil as the type for the second cell when CD45, CD66b, and CD193 CCR3 are each expressed in the second cell; identifying neutrophil as the type for the second cell when CD45 and CD66b are expressed in the second cell, and when CD193 CCR3 is not expressed in the second cell; and identifying basophil as the type for the second cell when CD45 and CD193 CCR3 are expressed in the second cell, and when CD66b is not expressed in the second cell.

Some embodiments further comprise: determining cell composition percentages of different types of cells in the biological sample based on the identified cell types.

In some embodiments, determining the cell composition percentages comprises: determining a first cell composition percentage for a first type of cell by determining a ratio between a number of cells in the plurality of cells identified as being of the first type and a total number of the cells in the plurality of cells.

In some embodiments, the subject has, is suspected of having, or is at risk of having cancer, and the method further comprises: identifying a treatment for the subject based on the determined cell composition percentages.

Some embodiments further comprise: administering the identified treatment to the subject.

In some embodiments, identifying the treatment for subject based on the determined cell composition percentages comprises: identifying an antibody anti-cancer agent for the subject when a cell composition percentage of peripheral blood mononuclear cells (PBMCs) is below a threshold.

In some embodiments, the antibody anti-cancer agent comprises ipilimumab.

In some embodiments, identifying the treatment for the subject based on the determined cell composition percentages comprises: determining a ratio between a cell composition percentage of CD8+PD-1+ cells and a cell composition percentage of CD4+PD-1; and identifying immune checkpoint blockade therapy for the subject when the determined ratio is above a threshold.

Some embodiments further comprise: comparing a cell composition percentage of the determined cell composition percentages to a range of cell composition percentages associated with a patient cohort; and identifying the subject as a member of the patient cohort based on a result of the comparing.

In some embodiments, the patient cohort comprises a healthy cohort, a cohort of patients with a disease, or a cohort of patients who have received a treatment.

Some embodiments further comprise: comparing a cell composition percentage of the determined cell composition percentages to a range of cell composition percentages associated with a study, wherein the study evaluates effectiveness of one or more treatments in treating a disease; and identifying a treatment for the subject based on a result of the comparing.

Some embodiments further comprise: identifying a first plurality of cell types present in a first subsample of the biological sample using flow cytometry performed using a first panel of markers and a first set of machine learning models, the first plurality of cell types including a first cell type; identifying a second plurality of cell types present in a second subsample of the biological sample using flow cytometry performed using a second panel of markers and a second set of machine learning models, the second plurality of cell types including the first cell type; determining, for each particular cell type of at least some of the cell types included in the first plurality of cell types and the second plurality of cell types, a cell composition percentage for the particular cell type, the determining comprising: determining a first number of cells of the particular cell type in the first plurality of cell types; determining a second number of cells of the particular cell type in the second plurality of cell types; normalizing the second number of cells of the particular cell type with respect to a number of cells of the first cell type included in the first plurality of cell types and a number of cells of the first cell type included in the second plurality of cell types; and determining the cell composition percentage for the particular cell type based on the first number of cells of the particular cell type and the normalized second number of cells of the particular cell type.

In some embodiments, the panel of markers comprises a first panel of markers, the plurality of cells includes a first plurality of cells and a second plurality of cells, the flow cytometry data includes first flow cytometry data for the first plurality of cells and second flow cytometry data for the second plurality of cells, the first flow cytometry data comprising flow cytometry measurements for markers in the first panel for each of at least some of the first plurality of cells and the second flow cytometry data comprising flow cytometry measurements for markers in a second panel of markers for each of at least some of the second plurality of cells, wherein the first panel of markers and the second panel of markers are different.

In some embodiments, identifying the types of cells of the plurality of cells using the set of machine learning models to obtain the respective plurality of cell types comprises: identifying types of cells of the first plurality of cells by processing the first flow cytometry data using a first set of machine learning models to obtain a first plurality of cell types, each machine learning model in the first set of machine learning models corresponding to a respective marker in the first panel of markers; and identifying types of cells of the second plurality of cells by processing the second flow cytometry data using a second set of machine learning models to obtain a second plurality of cell types, each machine learning model in the second set of machine learning models corresponding to a respective marker in the second panel of markers.

Some embodiments further comprise: determining, using the first plurality of cell types, a first plurality of cell counts at least in part by determining a respective number of cells of each of at least some of the first plurality of cell types; and determining, using the second plurality of cell types, a second plurality of cell counts at least in part by determining a respective number of cells of each of at least some of the second plurality of cell types.

Some embodiments further comprise determining a respective cell composition percentage for each of at least some cell types included in both the first plurality of cell types and the second plurality of cell types, the determining comprising combining at least some of the first plurality of cell counts and at least some of the second plurality of cell counts.

Some embodiments further comprise: determining a respective cell composition percentage for each of at least some cell types include in the first plurality of cell types and/or the second plurality of cell types, the determining comprising combining at least some of the first plurality of cell counts and at least some of the second plurality of cell counts based on flow cytometry data obtained by using a flow cytometry platform to measure beads in the biological sample.

In some embodiments, the flow cytometry measurements comprise fluorescent intensity values for at least some of the markers included in the panel of markers.

In some embodiments, the first flow cytometry measurements of the first cell comprise measurements for at least five markers in the panel of markers, processing the first flow cytometry measurements using the first machine learning model comprises providing the measurements for the at least five markers in the panel of markers as input to the first machine learning model, and processing the first flow cytometry measurements using the second machine learning model comprises providing the measurements for the at least five markers in the panel of markers as input to the second machine learning model.

In some embodiments, a compensation matrix was applied to the flow cytometry data, and some embodiments further comprise: processing at least a portion of the cytometry data using a trained neural network model to obtain an output indicative of a quality of the compensation matrix; and determining, based on the output indicative of the quality of the compensation matrix, whether to discard the cytometry data.

Some embodiments provide for a method for identifying types of cells present in a biological sample using flow cytometry performed using a panel of markers and a set of machine learning models each of which corresponds to a respective marker in the panel of markers. In some embodiments, the method comprises: using at least one computer hardware processor to perform: obtaining flow cytometry data for the biological sample, the biological sample previously obtained from a subject and comprising a plurality of cells, the flow cytometry data including flow cytometry measurements of cells in the plurality of cells that were obtained by a flow cytometry platform; and identifying cell types of at least 10,000 cells in the plurality of cells using the set of machine learning models to obtain a plurality of cell types, the set of machine learning models including at least five machine learning models corresponding respectively to at least five markers in the panel of markers, the identifying comprising, for each particular cell of the at least 10,000 cells, obtaining, from the flow cytometry data, respective flow cytometry measurements of the particular cell obtained by the flow cytometry platform, the respective flow cytometry measurements including measurements for the at least five markers; processing the respective flow cytometry measurements using each of the set of machine learning models to obtain multiple outputs each indicating a degree to which a respective marker of the at least five markers is expressed in the particular cell; and identifying the cell type for the particular cell using the multiple outputs.

Some embodiments provide for at least one non-transitory computer-readable storage medium storing processor-executable instructions that, when executed by at least one computer hardware processor, cause the at least one computer-hardware processor to perform a method for identifying types of cells present in a biological sample using flow cytometry performed using a panel of markers and a set of machine learning models each of which corresponds to a respective marker in the panel of markers, the method comprising: obtaining flow cytometry data for the biological sample, the biological sample previously obtained from a subject and comprising a plurality of cells, the flow cytometry data including flow cytometry measurements of cells in the plurality of cells that were obtained by a flow cytometry platform; and identifying cell types of at least 10,000 cells in the plurality of cells using the set of machine learning models to obtain a plurality of cell types, the set of machine learning models including at least five machine learning models corresponding respectively to at least five markers in the panel of markers, the identifying comprising, for each particular cell of the at least 10,000 cells, obtaining, from the flow cytometry data, respective flow cytometry measurements of the particular cell obtained by the flow cytometry platform, the respective flow cytometry measurements including measurements for the at least five markers; processing the respective flow cytometry measurements using each of the set of machine learning models to obtain multiple outputs each indicating a degree to which a respective marker of the at least five markers is expressed in the particular cell; and identifying the cell type for the particular cell using the multiple outputs.

Some embodiments provide for a system comprising: at least one computer hardware processor; and at least one non-transitory computer-readable storage medium storing processor-executable instructions that, when executed by the at least one computer hardware processor, cause the at least one computer hardware processor to perform a method for identifying types of cells present in a biological sample using flow cytometry performed using a panel of markers and a set of machine learning models each of which corresponds to a respective marker in the panel of markers, the method comprising: obtaining flow cytometry data for the biological sample, the biological sample previously obtained from a subject and comprising a plurality of cells, the flow cytometry data including flow cytometry measurements of cells in the plurality of cells that were obtained by a flow cytometry platform; and identifying cell types of at least 10,000 cells in the plurality of cells using the set of machine learning models to obtain a plurality of cell types, the set of machine learning models including at least five machine learning models corresponding respectively to at least five markers in the panel of markers, the identifying comprising, for each particular cell of the at least 10,000 cells, obtaining, from the flow cytometry data, respective flow cytometry measurements of the particular cell obtained by the flow cytometry platform, the respective flow cytometry measurements including measurements for the at least five markers; processing the respective flow cytometry measurements using each of the set of machine learning models to obtain multiple outputs each indicating a degree to which a respective marker of the at least five markers is expressed in the particular cell; and identifying the cell type for the particular cell using the multiple outputs.

Some embodiments provide for a method for identifying types of cells present in a biological sample using flow cytometry performed using a panel of markers and multiple machine learning models, the method comprising: using at least one computer hardware processor to perform: obtaining flow cytometry data for the biological sample, the biological sample previously-obtained from a subject and comprising a plurality of cells, the flow cytometry data including flow cytometry measurements obtained during respective flow cytometry events, the flow cytometry events corresponding to particular objects in the biological sample being measured by a flow cytometry platform, the flow cytometry events including a subset of events corresponding to cells in the biological sample being measured by the flow cytometry platform; and identifying types of cells of the plurality of cells using the multiple machine learning models to obtain a respective plurality of cell types, the multiple machine learning models including at least one first machine learning model and at least one second machine learning model different from the at least one first machine learning model, the identifying comprising, for each particular event in the subset of events, obtaining, from the flow cytometry data, flow cytometry measurements corresponding to the particular event; identifying an event type for the particular event by processing the flow cytometry measurements corresponding to the particular event using the at least one first machine learning model, the event type indicating whether the particular event corresponds to a cell being measured by the flow cytometry platform, or a bead being measured by the flow cytometry platform; when the identified event type indicates that the particular event corresponds to the cell being measured by the flow cytometry platform, identifying a type of the cell by processing the flow cytometry measurements corresponding to the particular event using the at least one second machine learning model.

Some embodiments provide for at least one non-transitory computer-readable storage medium storing processor-executable instructions that, when executed by at least one computer hardware processor, cause the at least one computer hardware processor to perform a method for identifying types of cells present in a biological sample using flow cytometry performed using a panel of markers and multiple machine learning models, the method comprising: obtaining flow cytometry data for the biological sample, the biological sample previously-obtained from a subject and comprising a plurality of cells, the flow cytometry data including flow cytometry measurements obtained during respective flow cytometry events, the flow cytometry events corresponding to particular objects in the biological sample being measured by a flow cytometry platform, the flow cytometry events including a subset of events corresponding to cells in the biological sample being measured by the flow cytometry platform; and identifying types of cells of the plurality of cells using the multiple machine learning models to obtain a respective plurality of cell types, the multiple machine learning models including at least one first machine learning model and at least one second machine learning model different from the at least one first machine learning model, the identifying comprising, for each particular event in the subset of events, obtaining, from the flow cytometry data, flow cytometry measurements corresponding to the particular event; identifying an event type for the particular event by processing the flow cytometry measurements corresponding to the particular event using the at least one first machine learning model, the event type indicating whether the particular event corresponds to a cell being measured by the flow cytometry platform, or a bead being measured by the flow cytometry platform; when the identified event type indicates that the particular event corresponds to the cell being measured by the flow cytometry platform, identifying a type of the cell by processing the flow cytometry measurements corresponding to the particular event using the at least one second machine learning model.

Some embodiments provide for a system comprising: at least one computer hardware processor; and at least one non-transitory computer-readable storage medium storing processor-executable instructions that, when executed by the at least one computer hardware processor, cause the at least one computer hardware processor to perform a method for identifying types of cells present in a biological sample using flow cytometry performed using a panel of markers and multiple machine learning models, the method comprising: obtaining flow cytometry data for the biological sample, the biological sample previously-obtained from a subject and comprising a plurality of cells, the flow cytometry data including flow cytometry measurements obtained during respective flow cytometry events, the flow cytometry events corresponding to particular objects in the biological sample being measured by a flow cytometry platform, the flow cytometry events including a subset of events corresponding to cells in the biological sample being measured by the flow cytometry platform; and identifying types of cells of the plurality of cells using the multiple machine learning models to obtain a respective plurality of cell types, the multiple machine learning models including at least one first machine learning model and at least one second machine learning model different from the at least one first machine learning model, the identifying comprising, for each particular event in the subset of events, obtaining, from the flow cytometry data, flow cytometry measurements corresponding to the particular event; identifying an event type for the particular event by processing the flow cytometry measurements corresponding to the particular event using the at least one first machine learning model, the event type indicating whether the particular event corresponds to a cell being measured by the flow cytometry platform, or a bead being measured by the flow cytometry platform; when the identified event type indicates that the particular event corresponds to the cell being measured by the flow cytometry platform, identifying a type of the cell by processing the flow cytometry measurements corresponding to the particular event using the at least one second machine learning model.

Some embodiments further comprise: selecting the at least one second machine learning model based on data indicative of the panel of markers.

In some embodiments, selecting the at least one second machine learning model from the group of machine learning models listed in Tables 1A, 2A, 3-7A, 8A, and 9-10A, wherein the machine learning models listed in Tables 1A, 2A, 3-7A, 8A, and 9-10A each correspond to a respective panel of markers.

In some embodiments, selecting the at least one second machine learning model comprises selecting a machine learning model trained to predict, based on flow cytometry measurements, a type for a cell.

In some embodiments, the machine learning model trained to predict the type for a cell is a multiclass classifier.

In some embodiments, selecting the at least one second machine learning model comprises selecting a set of machine learning models, each machine learning model in the set of machine learning models trained to predict a degree to which a marker in the panel of markers is expressed in a cell.

In some embodiments, a machine learning model in the set of machine learning models is a binary classifier.

In some embodiments, the at least one first machine learning model is a multiclass classifier, and the at least one second machine learning model comprises a binary classifier and/or a multiclass classifier.

In some embodiments, the at least one first machine learning model comprises a decision tree classifier or a gradient boosted decision tree classifier.

In some embodiments, the at least one first machine learning model comprises a gradient-boosted decision tree classifier.

In some embodiments, the gradient-boosted decision tree classifier comprises an ensemble of decision tree classifiers.

In some embodiments, the gradient-boosted decision tree classifier is implemented using LightGBM, XGBoost, or CatBoost.

In some embodiments, identifying the type for the cell by processing the flow cytometry measurements using the at least one second machine learning model comprises: processing the flow cytometry measurements using at least some machine learning models in the set of machine learning models to obtain outputs indicating a degree to which each of at least some markers in the panel of markers are expressed in the cell; and identifying the type for the cell based on the outputs indicating the degree to which each of the at least some markers in the panel of markers are expressed in the cell.

In some embodiments, the subset of events comprises at least 100 events, at least 500 events, at least 1,000 events, at least 2,500 events, at least 5,000 events, at least 10,000 events, at least 25,000 events, at least 75,000 events, at least 100,000 events, at least 150,000 events, at least 200,000 events, or at least 500,000 events.

Some embodiments further comprise: determining cell composition percentages of different types of cells in the biological sample based on the identified plurality of cell types.

In some embodiments, determining the cell composition percentages comprises: determining a first cell composition percentage for a first type of cell by determining a ratio between a number of cells in the plurality of cells identified as being of the first type and a total number of the cells in the plurality of cells.

In some embodiments, the subject has, is suspected of having, or is at risk of having cancer, and wherein the method further comprises: identifying a treatment for the subject based on the determined cell composition percentages.

Some embodiments further comprise: administering the identified treatment to the subject.

In some embodiments, identifying the treatment for subject based on the determined cell composition percentages comprises: identifying an antibody anti-cancer agent for the subject when a cell composition percentage of peripheral blood mononuclear cells (PBMCs) is below a threshold.

In some embodiments, the antibody anti-cancer agents comprises ipilimumab.

In some embodiments, identifying the treatment for the subject based on the determined cell composition percentages comprises: determining a ratio between a cell composition percentage of CD8+PD-1+ cells and a cell composition percentage of CD4+PD-1; and identifying immune checkpoint blockade therapy for the subject when the determined ratio is above a threshold.

Some embodiments further comprise: comparing a cell composition percentage of the determined cell composition percentages to a range of cell composition percentages associated with a patient cohort; and identifying the subject as a member of the patient cohort based on a result of the comparing.

In some embodiments, the patient cohort comprises a healthy cohort, a cohort of patients with a disease, or a cohort of patients who have received a treatment.

Some embodiments further comprise: comparing a cell composition percentage of the determined cell composition percentages to a range of cell composition percentages associated with a study, wherein the study evaluates effectiveness of one or more treatments in treating a disease; and identifying a treatment for the subject based on a result of the comparing.

Some embodiments further comprise: identifying a first plurality of cell types present in a first subsample of the biological sample using flow cytometry performed using a first panel of markers, the first plurality of cell types including a first cell type; identifying a second plurality of cell types present in a second subsample of the biological sample using flow cytometry performed using a second panel of markers, the second plurality of cell types including the first cell type; and determining, for each particular cell type of at least some of the cell types included in the first plurality of cell types and the second plurality of cell types, a cell composition percentage for the particular cell type, the determining comprising: determining a first number of cells of the particular cell type in the first plurality of cells; determining a second number of cells of the particular cell type in the second plurality of cells; normalizing the second number of cells of the particular type with respect to a number of cells of the first cell type included in the first plurality of cell types and a number of cells of the first cell type included in the second plurality of cell types; and determining the cell composition percentage for the particular cell type based on the first number of cells of the particular cell type and the normalized second number of cells of the particular cell type.

In some embodiments, the panel of markers comprises a first panel of markers, the plurality of cells includes a first plurality of cells and a second plurality of cells, the flow cytometry data includes first flow cytometry data for the first plurality of cells and second flow cytometry data for the second plurality of cells, the first flow cytometry data comprising flow cytometry measurements for markers in the first panel for each of at least some of the first plurality of cells and the second flow cytometry data comprising flow cytometry measurements for markers in a second panel of markers for each of at least some of the second plurality of cells, wherein the first panel of markers and the second panel of markers are different.

In some embodiments, identifying the types of cells of the plurality of cells using multiple machine learning models to obtain the respective plurality of cell types comprises: identifying types of cells of the first plurality of cells by processing the first flow cytometry data using a first subset of the multiple machine learning models to obtain a first plurality of cell types; and identifying types of cells of the second plurality of cells by processing the second flow cytometry data using a second subset of the multiple machine learning models to obtain a second plurality of cell types.

Some embodiments further comprise: determining, using the first plurality of cell types, a first plurality of cell counts at least in part by determining a respective number of cells of each of at least some of the first plurality of cell types; and determining, using the second plurality of cell types, a second plurality of cell counts at least in part by determining a respective number of cells of each of at least some of the second plurality of cell types.

Some embodiments further comprise: determining a respective cell composition percentage for each of at least some cell types included in both the first plurality of cell types and the second plurality of cell types, the determining comprising combining at least some of the first plurality of cell counts and at least some of the second plurality of cell counts.

Some embodiments further comprise: determining a respective cell composition percentage for each of at least some cell types include in the first plurality of cell types and/or the second plurality of cell types, the determining comprising combining at least some of the first plurality of cell counts and at least some of the second plurality of cell counts based on flow cytometry data obtained by using a flow cytometry platform to measure beads in the biological sample.

In some embodiments, the flow cytometry measurements comprise fluorescent intensity values for at least some of the markers included in the panel of markers.

In some embodiments, the flow cytometry measurements corresponding to the particular event comprise measurements for at least five markers in the panel of markers, processing the flow cytometry measurements corresponding to the particular event using the at least one first machine learning model comprises providing the measurements for the at least five markers in the panel of markers as input to the at least one first machine learning model, and processing the flow cytometry measurements corresponding to the particular event using at least the second machine learning model comprises providing the measurements for the at least five markers in the panel of markers as input to the second machine learning model.

### BRIEF DESCRIPTION OF DRAWINGS

FIG. 1A is a diagram depicting an illustrative technique for determining a respective type for one or more events based on cytometry data, according to some embodiments of the technology described herein.

FIG. 1B is a table showing example cytometry data for multiple events corresponding to cells and/or particles in a biological sample, according to some embodiments of the technology described herein.

FIG. 1C is a table showing example cytometry data, including example marker values for multiple events, according to some embodiments of the technology described herein.

FIG. 1D is a block diagram of a system 150 including an example computing device and software, according to some embodiments of the technology described herein.

FIG. 2A is a flowchart of an illustrative process 200 for identifying types of cells present in a biological sample using multiple machine learning models, according to some embodiments of the technology described herein.

FIG. 2B is a flowchart depicting an illustrative process 250 for identifying types of cells present in a biological sample using a set of machine learning models each of which corresponds to a respective marker in a panel of markers, according to some embodiments of the technology described herein.

FIG. 3A is a diagram of an illustrative technique for identifying types of cells present in a biological sample using multiple machine learning models, according to some embodiments of the technology described herein.

FIG. 3B is a diagram of an illustrative technique for identifying an event type for an event using one or more machine learning models, according to some embodiments of the technology described herein.

FIG. 3C is a diagram of an illustrative technique for identifying a type for a cell using one or more machine learning models, according to some embodiments of the technology described herein.

FIG. 3D is a diagram of an illustrative technique for identifying a type for a cell using a set of machine learning models each of which corresponds to a marker in a panel of markers, according to some embodiments of the technology described herein.

FIG. 4A is a flowchart of an illustrative process 400 for identifying a subject as a member of a patient cohort, according to some embodiments of the technology described herein.

FIG. 4B is a flowchart of an illustrative process 420 for determining cell composition percentages based on cell counts determined for different subsamples of a biological sample, according to some embodiments of the technology described herein.

FIG. 4C is a flowchart of an illustrative process 460 for normalizing cell composition percentages with respect to hierarchical relationships between cell types, according to some embodiments of the technology described herein.

FIG. 4D is a flowchart of an illustrative process 480 for determining cell composition percentages based on marker composition, according to some embodiments of the technology described herein.

FIG. 5A depicts an illustrative example for determining cell composition percentages based on cell types determined using cytometry data obtained by performing cytometry using a single panel of markers, according to some embodiments of the technology described herein.

FIG. 5B, FIG. 5C, FIG. 5D, and FIG. 5E each depict an illustrative example for determining cell composition percentages based on cell types determined using cytometry data obtained by performing cytometry using different panels, according to some embodiments of the technology described herein.

FIGS. 6A-6E are screenshots of an example report indicating information for multiple cell populations of particular types in a biological sample, according to some embodiments of the technology described herein.

FIG. 7 is a flowchart of an illustrative process 700 for training one or more machine learning models, according to some embodiments of the technology described herein.

FIG. 8A shows a plot used to identify duplicates in cytometry data, according to some embodiments.

FIG. 8B shows the results of clustering cytometry data that has not undergone a noise transformation, according to some embodiments of the technology described herein.

FIG. 8C shows the results of clustering cytometry data that has undergone the noise transformation, according to some embodiments of the technology described herein.

FIG. 8D shows the distribution of marker intensities resulting from cytometry data that has not undergone the noise transformation, according to some embodiments of the technology described herein.

FIG. 8E shows the distribution of marker intensities resulting from cytometry data that has undergone the noise transformation, according to some embodiments of the technology described herein.

FIG. 8F and FIG. 8G are plots used for identifying different types of events based on marker values, according to some embodiments of the technology described herein.

FIG. 8H and FIG. 8I are plots showing event clusters that were labelled to indicate the event type, according to some embodiments of the technology described herein.

FIG. 8J is a plot used to identify outliers based on marker intensity values, according to some embodiments of the technology described herein.

FIG. 8K is a plot used to distinguish between event populations based on event density, according to some embodiments of the technology described herein.

FIG. 8L is a plot showing event clusters that were labelled to indicate event type, according to some embodiments of the technology described herein.

FIG. 9 is a plot used to distinguish between two cell populations based on intensities of a combination of different markers, according to some embodiments of the technology described herein.

FIG. 10A and FIG. 10B are examples comparing uncompensated cytometry data to compensated cytometry data, according to some embodiments of the technology described herein.

FIG. 10C shows examples of cytometry data obtained using different compensation settings, according to some embodiments of the technology described herein.

FIG. 10D shows an example of compensation artifacts, according to some embodiments of the technology described herein.

FIG. 10E, FIG. 10F, FIG. 10G, and FIG. 10H show examples of results of applying different compensation techniques, according to some embodiments of the technology described herein.

FIG. 11A is a flowchart of an illustrative process for determining a quality of compensation applied to cytometry data, according to some embodiments of the technology described herein.

FIG. 11B shows examples of two-dimensional distributions of marker pairs labeled as having a compensation quality that is greater than or equal to a threshold quality, according to some embodiments of the technology described herein.

FIG. 11C shows examples of two-dimensional distributions of marker pairs labeled as having a compensation quality that is less than or equal to the threshold quality, according to some embodiments of the technology described herein.

FIG. 11D is an example of an architecture of a neural network used to determine a quality of compensation applied to cytometry data, according to some embodiments of the technology described herein.

FIG. 12 depicts an illustrative implementation of a computer system that may be used in connection with some embodiments of the technology described herein.

### DETAILED DESCRIPTION

Understanding the cellular makeup of a biological sample is important in many different applications. For example, understanding the cellular makeup of a biological sample of a tumor provides insight into the tumor microenvironment (TME), which is important for diagnostic, prognostic, and treatment purposes. For example, understanding the makeup of the TME, including the types of immune cells present in the TME, helps to guide therapeutic decision making.

The types of cells in a biological sample may be identified using a variety of techniques. For example, cellular deconvolution techniques may be used to identify the cellular makeup of a sample from RNA sequencing data. Example cellular deconvolution techniques are described in U.S. Pat. No. 11,315,658, filed Mar. 12, 2021, and entitled “SYSTEMS AND METHODS FOR DECONVOLUTION OF EXPRESSION DATA,” and U.S. Patent Publication No. 2022/0372580, filed Apr. 29, 2022, and entitled “MACHINE LEARNING TECHNIQUES FOR ESTIMATING TUMOR EXPRESSION IN COMPLEX TUMOR TISSUE,” each of which is incorporated by reference herein in its entirety.

As described above, cytometry provides another approach to characterizing the cellular composition of a sample. However, conventional techniques of using cytometry to identify cell types in a sample have drawbacks and may be improved upon.

For example, some conventional techniques of using cytometry to identify cell types in a sample involves manually analyzing the generated cytometry data. Such techniques include plotting cytometry measurements of cells in a sample in a series of two-dimensional plots and manually identifying regions of interest in each plot, which is a technique commonly referred to as “gating”. To identify such regions of interest, an operator defines boundaries around groups of plotted points. The operator then manually labels the identified regions as corresponding to particular cell types. Cells having cytometry measurements falling within a particular region of interest are identified as being of the cell type with which the particular region of interest was labelled.

One problem with such techniques is that identifying a region of interest is a subjective procedure with poor reproducibility because different operators may make (and, in practice, often do make) different decisions about where to place boundaries. For example, some operators may place more expansive boundaries, including more points, while other operators may place more restrictive boundaries, including fewer points. As a result, there is variability in the data used to determine types for individual cells, leading to variable results of such analyses. This issue becomes more pronounced when analyzing large volumes of cytometry data, since this involves generating and identifying regions of interest in a larger number of plots, leading to greater overall variation in the data used for cell type determination. As a result, different operators may classify the same cell differently (e.g., one operator may classify a cell as being of one type and another operator may classify the cell as being a different type). Consequently, different operators will produce different estimates of cell population percentages (e.g., estimates of the proportion of each of one or more cell types in the overall cell population). Moreover, manual analysis of data is time-consuming, expensive, and inefficient, which poses challenges for large-scale studies, such as patient cohort and drug screening studies, which generate complex, multidimensional datasets.

There have been attempts to automate the above-described manual methods. For example, some conventional techniques involve using unsupervised machine learning clustering techniques in order to automatically cluster cytometry measurements into clusters corresponding to different cell populations. After the clusters are formed, each cluster may be labelled (e.g., manually or automatically) as corresponding to a particular cell type. However, such unsupervised machine learning methods require input specifying the number of clusters into which to group the cytometry data. The specification of the number of clusters is subjective and is typically based on an operator's estimate of the number of cell populations in the sample. Such estimates are often wrong and lead to inaccurate estimates of cell populations present in the sample. If the specified number of clusters is too low, then the clustering algorithm will not accurately account for relatively small cell populations in the sample, but rather group them into other, larger populations of cells. If the specified number of clusters is too high, then the clustering algorithm will incorrectly split one population into multiple different populations. Consequently, conventional clustering algorithms may assign cells of the same type into different cell populations depending on operator input specifying the number of clusters.

Inaccurately and unreliably determining cell types for a sample, using conventional manual or automated cytometry analysis techniques, necessarily affects the accuracy and reliability of estimating the cellular composition of the sample overall. The cellular composition of a sample may be estimated based on the relative numbers of cells of each cell type in the sample. When the estimated sizes of cell populations are inaccurate, the estimated cellular composition will not accurately reflect the relative number of cells in each cell population. This is particularly problematic in some medical applications. For example, as explained above, the cellular composition may be used to diagnose a subject with a disease or predict whether the subject will respond to a particular treatment. When the estimated cellular composition is inaccurate, the subject may be diagnosed with the wrong disease, treated with an inappropriate therapy, or not treated with a therapy that would have been helpful. For example, a relatively large cell composition percentage of CD4+ cells may indicate that a patient will respond well to Rituximab. If conventional cytometry analysis techniques incorrectly determine the relative proportion of CD4+ cells in a sample, then the patient may be given Rituxmab when they may respond negatively (or not at all) or may not be given Rituxmab when, in fact, that treatment would have been helpful.

Accordingly, the inventors have developed techniques for more accurately, reliably, and efficiently identifying types for cells included in a biological sample based on cytometry data that address the above-described problems of conventional techniques. The techniques include processing cytometry data using multiple machine learning models to identify types of cells present in a biological sample. The cytometry data may include cytometry measurements (e.g., marker values) obtained during respective cytometry events (“events”), which correspond to particular types of objects (e.g., a cell, a bead, debris, etc.) in a biological sample being measured by a cytometry platform (e.g., a flow or a mass cytometry platform).

In some embodiments, using multiple machine learning models to identify types of cells present in a biological sample may be performed in two stages. In the first stage, a machine learning model may be used to identify the cytometry events during which cells (rather than debris or beads) were measured by the cytometry platform. In the second stage, one or more other machine learning models may be used to process cytometry data obtained during “cell” events to identify the types of cells measured.

In some embodiments, in the first stage, a multiclass classifier trained to predict the event type from among multiple event types may be used to assign event types to individual cytometry events. For example, the event type may indicate whether the event corresponds to a cell, a bead, or debris being measured by the cytometry platform. The multi-class classifier may be a gradient-boosted decision tree classifier, in some embodiments.

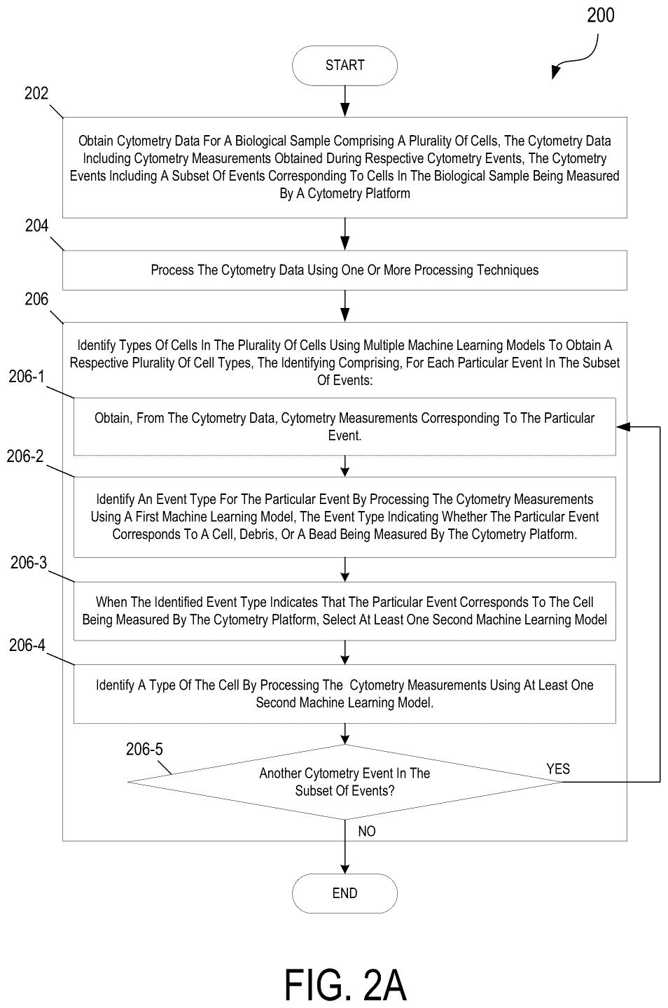

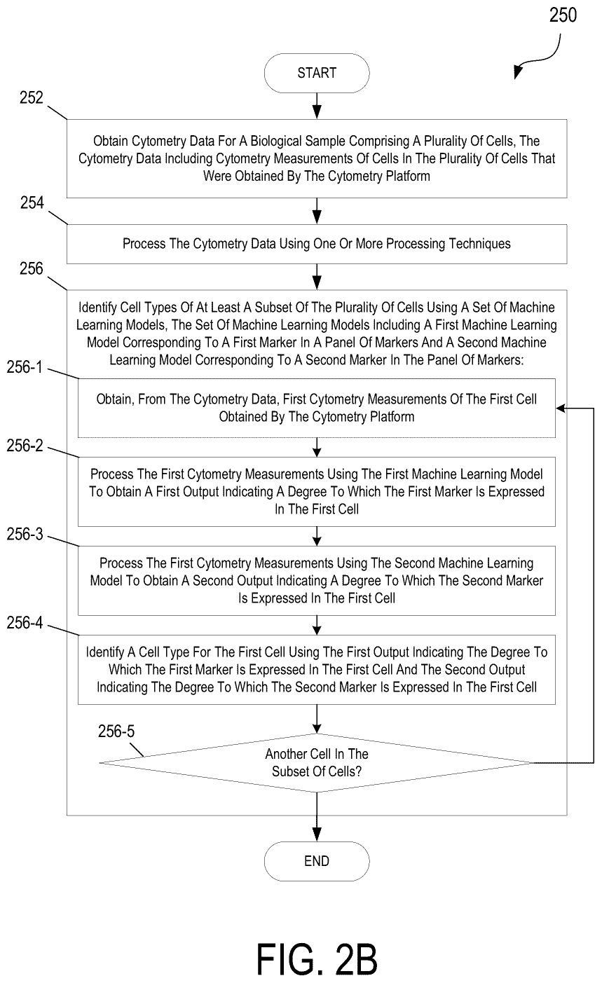

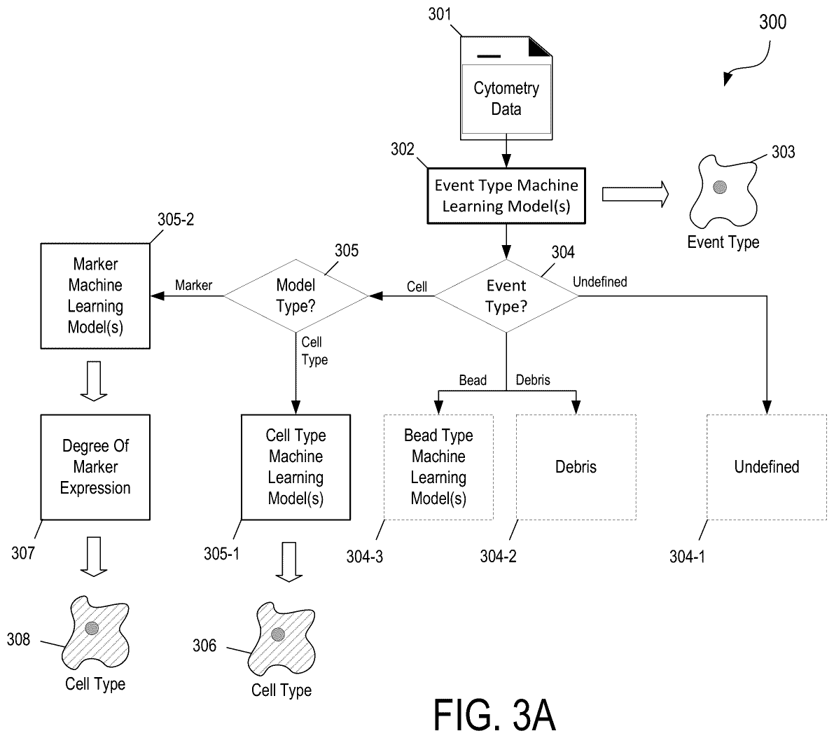

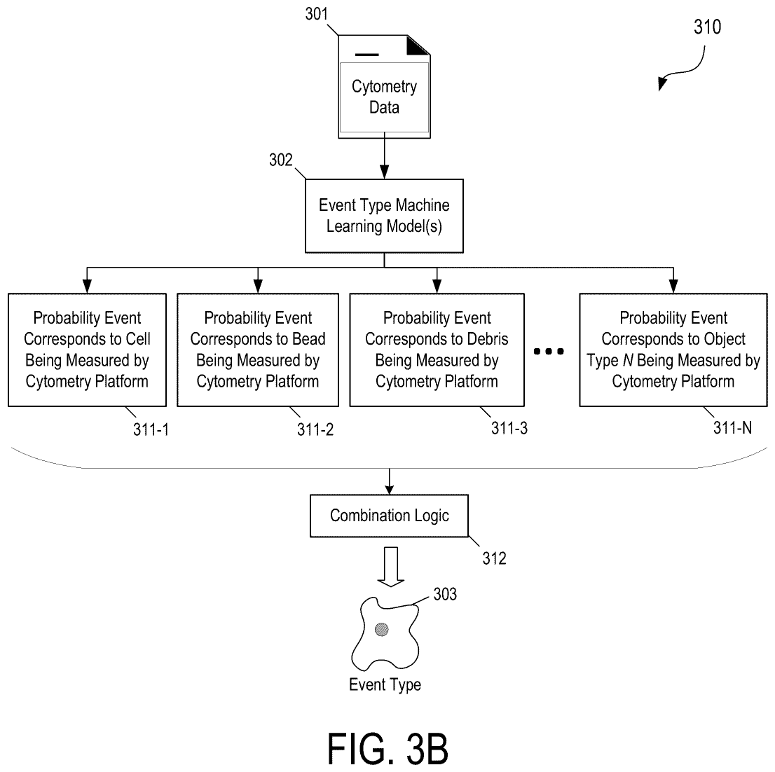

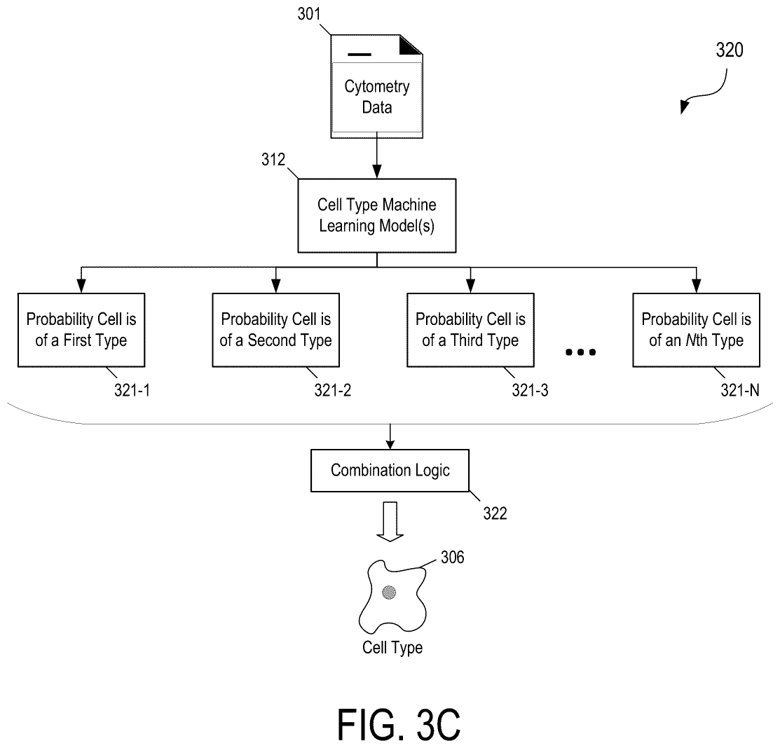

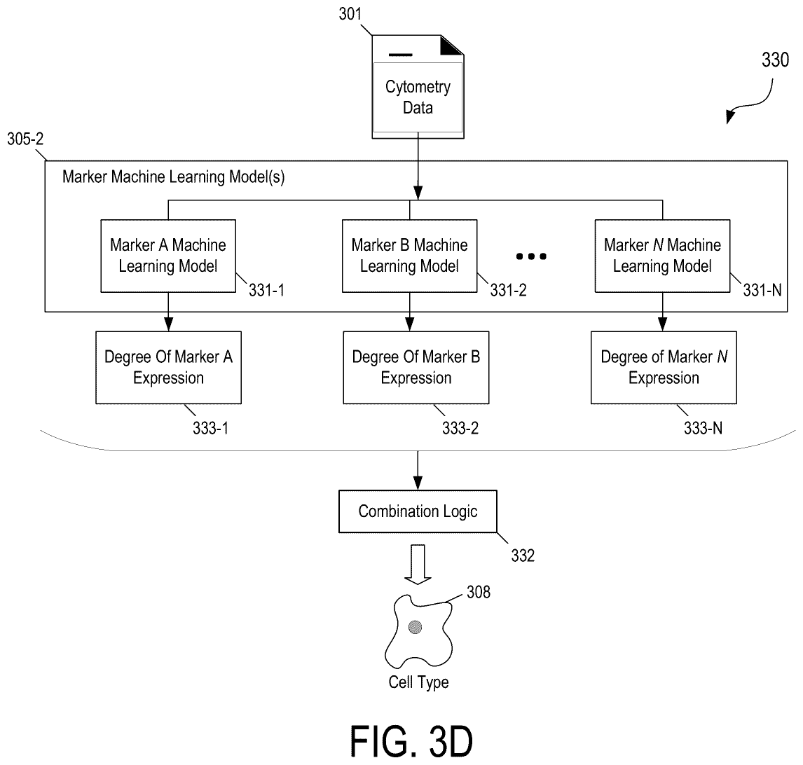

Different machine learning approaches may be taken in the second stage. For example, in some embodiments, cytometry data obtained during a “cell” event may be processed by a single multi-class classification model (e.g., a random forest model or a gradient-boosted machine learning model) to predict a type for the cell measured during the event. Aspects of this type of approach are described herein including at least with respect to FIGS. 2A, 3A, and 3C.

Alternatively, in some embodiments, identifying the cell type includes predicting the degree to which certain markers are expressed in the cell, and identifying the cell type based on the predicted degrees of marker expression. In some embodiments, each of multiple machine learning models (e.g., binary classifiers) may be used to process the cytometry data to obtain an output indicative of the degree to which a particular marker is expressed. For example, the output may indicate whether or not the particular marker is expressed. In some embodiments, identifying the cell type for the cell includes identifying a cell type associated with the predicted marker expression. Aspects of this type of approach are described herein including at least with respect to FIGS. 2A-2B, 3A, and 3D.

Furthermore, the inventors have developed techniques for more accurately estimating cell composition percentages of multiple cell types in the biological sample. In some embodiments, this includes splitting cells in the same biological sample into two or more subgroups, termed “subsamples,” and fluorescently labelling them with different panels of markers. Due to the limited number of fluorochromes with distinct emission spectra, the markers are split into the different panels, rather than labeling the markers using fluorochromes having overlapping emission spectra.

Obtaining cytometry measurements for different markers in different panels allows for the identification of different cell types. For example, one panel of markers may be used to identify cells of a first type in a first subsample, while a different panel of markers may be used to identify cells of a different type in a different subsample. Accordingly, in some embodiments, cytometry data obtained for a particular subsample may be used to estimate cell counts for the cell types identified in that particular subsample. For example, for cells identified as T cells in a first subsample, a cell count for T cells may be estimated. However, due to variability among the different subsamples, the percentage of T cells in the first subsample may not (and likely does not) accurately reflect the percentage of T cells in the overall biological sample. Accordingly, the inventors have developed techniques for normalizing cell counts determined for different subsamples, such that they are invariant to the different subsamples. In some embodiments, the normalized cell counts may then be used to determine cell composition percentages for cell types in the biological sample.

In some embodiments, the cell counts are normalized based on cell counts determined using a “leader” panel of markers. In some embodiments, a leader panel may include a panel of markers used to obtain cytometry measurements that can be used to distinguish among particular cell types. For example, the particular cell types may include one or more cell types that are common between the leader panel and a non-leader panel. For example, the leader panel and a first non-leader panel may each be used to identify T cells, while the leader panel and a second non-leader panel may each be used to identify neutrophils. In some embodiments, cell counts determined using non-leader panels are normalized based on cell counts determined for the common cell type. For example, the cell counts may be normalized based on a ratio between the cell count determined for the common cell type using the leader panel and the cell count determined for the common cell type using the non-leader panel.

Additionally, or alternatively, in some embodiments, the techniques developed by the inventors include normalizing the cell counts based on beads included in the subsamples. For example, in some embodiments, a known concentration of beads is added to the biological sample before it is divided into subsamples. When a subsample is processed using cytometry, cytometry measurements may be obtained for the beads and can used to estimate a number of beads in the particular subsample. In some embodiments, the cell counts determined for that particular subsample may be normalized based on the number of beads and the known concentration of beads.

In some embodiments, once normalized, the cell counts can be directly compared and combined to more accurately determine cell composition percentages for different cell types in the biological sample. This is an improvement over conventional cytometry techniques, which do not involve normalizing and/or combining cell counts determined using different panels of markers, or determining cell composition percentages based on such normalized and/or combined cell counts. As a result, the cell composition percentages determined using the techniques described herein improve upon conventional machine learning techniques for processing cytometry data because such conventional techniques (e.g., unsupervised clustering) do not fuse data from multiple different panels of markers.

The techniques developed by the inventors offer an improvement over conventional cell type identification techniques by improving the accuracy and reproducibility of the cell type identification results. In particular, in some embodiments, one or more machine learning models are used to identify a type for a cell in a biological sample based on cytometry data. Using one or more machine learning models in this way eliminates the subjective processes of conventional techniques, including the processes that rely on a human operator to estimate the number of cell populations in a sample and to identify cell types of resulting cell populations. As described above, such conventional techniques can produce overinclusive or underinclusive results, leading to inaccurate, inconsistent identification of cell types. By contrast, the systems and methods described herein, through the use of the one or more machine learning models, produce more accurate and reproducible results, resulting in an improvement over conventional techniques for identifying cell types. Such an improvement is important to applications of cytometry where cell count and/or cell composition percentages are used to inform diagnosis and/or have treatment implications.

Following below are descriptions of various concepts related to, and embodiments of, techniques for determining types of one or more cells in a biological sample. It should be appreciated that various aspects described herein may be implemented in any of numerous ways, as the techniques are not limited in any particular manner of implementation. Example details of implementations are provided herein solely for illustrative purposes. Furthermore, the techniques disclosed herein may be used individually or in any suitable combination, as aspects of the technology described herein are not limited to the use of any particular technique or combination of techniques.

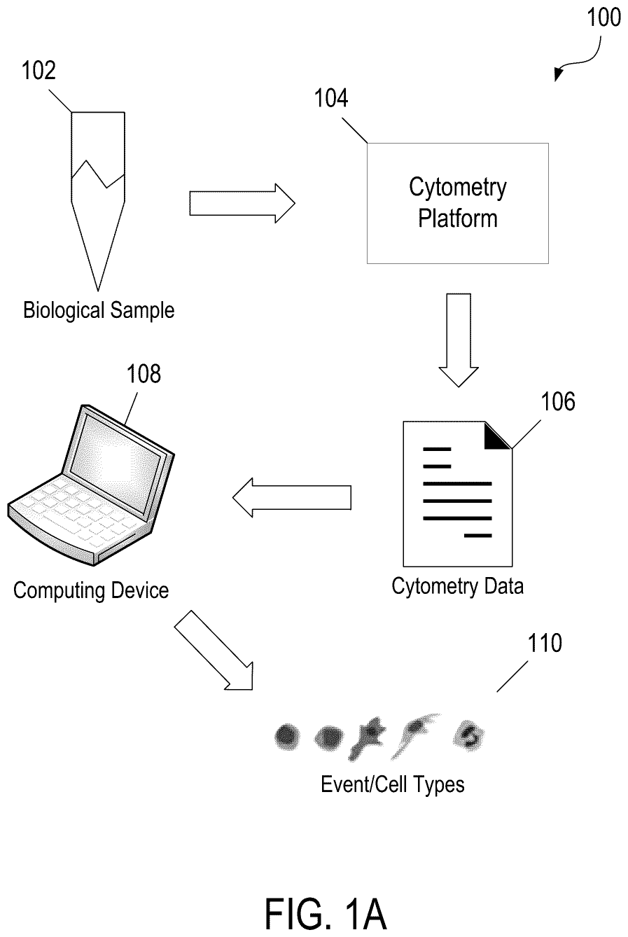

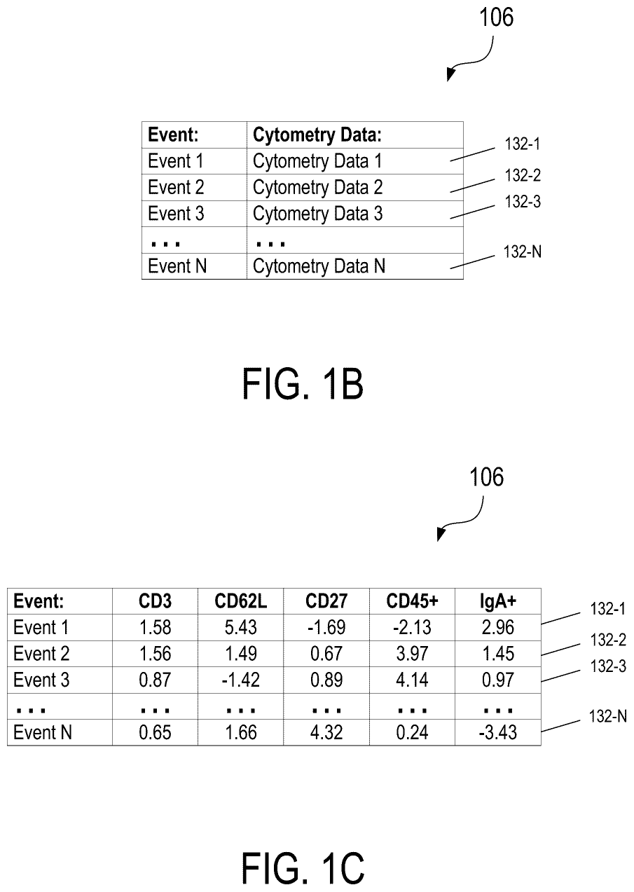

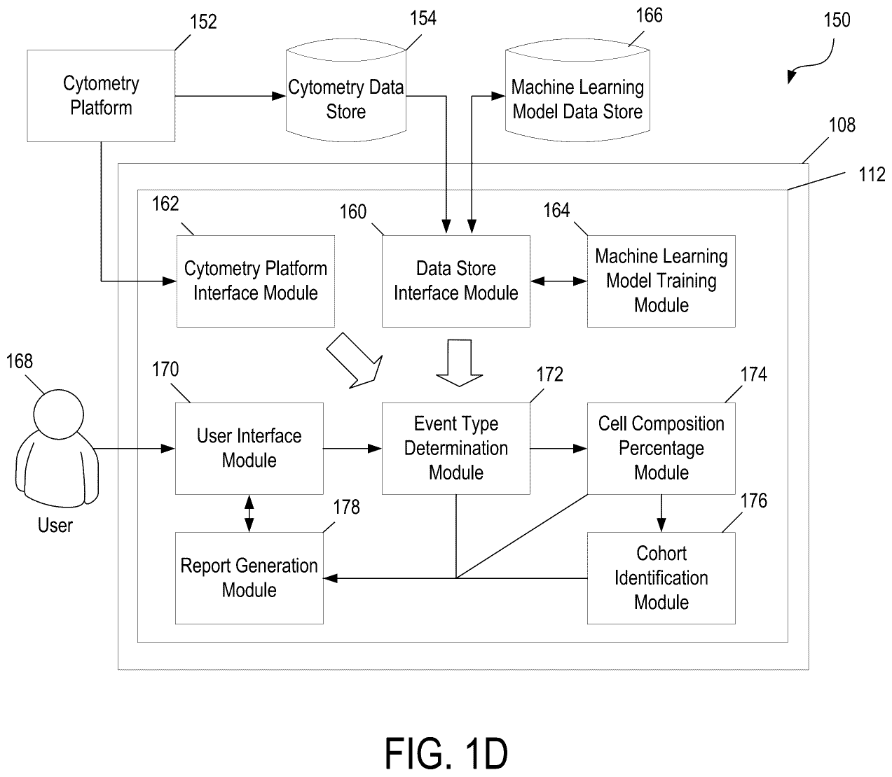

FIG. 1A depicts an illustrative technique 100 for determining a respective type 110 for each of one or more events. As described herein, in some embodiments, an event corresponds to obtaining measurements for an object in a biological sample 102. Obtaining measurements for an object, in some embodiments, includes obtaining cytometry data, such as cytometry data 106. In some embodiments, the cytometry data 106 is obtained by processing the object using a cytometry platform 104. Additionally, or alternatively, the cytometry data 106 may have been previously-obtained using a cytometry platform 104. A respective type 110 for each of one or more events is determined by processing the cytometry data 106 using computing device 108. In some embodiments, the computing device 108 may be part of cytometry platform 104. In other embodiments, the computing device 108 may be separate from the cytometry platform 104 and may receive cytometry data 106, directly or indirectly, from the cytometry platform 104.

In some embodiments, aspects of the illustrative technique 100 may be implemented in a clinical or laboratory setting. For example, aspects of the illustrative technique 100 may be implemented on a computing device 108 that is located within the clinical or laboratory setting. In some embodiments, the computing device 108 may directly obtain cytometry data 106 from a cytometry platform 104 and/or from a user (e.g., by the user uploading the cytometry data 106 and/or by interacting with computing device 108) within the clinical or laboratory setting. For example, a computing device 108 included within the cytometry platform 104 may directly obtain cytometry data 106 from the cytometry platform 104. In some embodiments, the computing device 108 may indirectly obtain cytometry data 106 from another device (e.g., cytometry platform 104 and/or another computing device) within the clinical or laboratory setting. For example, the computing device 108 may obtain cytometry data 106 via a communication network, such as Internet or any other suitable network, as aspects of the technology described herein are not limited to any particular communication network.

In some embodiments, aspects of the illustrative technique 100 may be implemented in a setting that is located external to a clinical or laboratory setting. In this case, the computing device 108 may indirectly obtain cytometry data 106 from the cytometry platform 104 and/or another computing device located within or externally to a clinical or laboratory setting. For example, the cytometry data 106 may be provided to the computing device 108 via a communication network, such as Internet or any other suitable network, as aspects of the technology described herein are not limited to any particular communication network.

As shown in FIG. 1A, the technique 100 involves processing a biological sample 102 using a cytometry platform 104, which produces cytometry data 106. The biological sample 102 may be obtained from a subject having, suspected of having, or at risk of having cancer and/or an immune-related disease and/or condition. The biological sample 102 may be obtained by performing a biopsy or by obtaining a blood sample, a salivary sample, or any other suitable biological sample from the subject. The biological sample 102 may include diseased tissue (e.g., cancerous), and/or healthy tissue. In some embodiments, the origin or preparation methods of the biological sample may include any of the embodiments described herein including at least in the “Biological Samples” section.

In some embodiments, the cytometry platform 104 is configured to process the biological sample 102 to produce cytometry data 106. In some embodiments, the cytometry platform 104 is a flow cytometry platform, a mass cytometry platform, or any other suitable platform configured to perform cytometry, as aspects of the technology described herein are not limited in this respect. In some embodiments, flow cytometry techniques may include any of the embodiments described herein including with respect to the “Flow Cytometry” section. In some embodiments, mass cytometry techniques may include any of the embodiments described herein including with respect to the “Mass Cytometry” section.

In some embodiments, the cytometry data 106 includes cytometry data for each of one or more events. Each event may correspond to obtaining cytometry measurements for an object in the biological sample using a cytometry platform. In some embodiments, the objects include cells, particles, and/or undefined objects. In some embodiments, the particles include beads, debris, and/or doublets. “Beads,” or calibration beads, are particles of a known concentration that can be mixed with a known volume of a biological sample, prior to being processed by a flow cytometer or a mass cytometer. The proportion of beads detected and identified in cytometry data for a subsample can be used to determine the number of cells in the subsample and/or the number of cells of a particular type in the subsample. A “doublet” is a pair of two independent particles or cells that are processed and classified by the cytometry platform as a single particle. This occurs when two cells or particles pass through the cytometry platform very close to one another. Cytometry data is further described herein including at least with respect to FIGS. 1B-1C.

In some embodiments, the cytometry data 106 is processed using computing device 108. In some embodiments, computing device 108 can be one or multiple computing devices of any suitable type. For example, the computing device 108 may be a portable computing device (e.g., a laptop, a smartphone) or a fixed computing device (e.g., a desktop computer, a server). When computing device 108 includes multiple computing devices, the device(s) may be physically co-located (e.g., in a single room) or distributed across multiple physical locations. In some embodiments, the computing device 108 may be part of a cloud computing infrastructure. In some embodiments, one or more computer(s) 108 may be co-located in a facility operated by an entity (e.g., a hospital, a research institution). In some embodiments, the one or more computing device(s) 108 may be physically co-located with a medical device, such as a cytometry platform 104. For example, a cytometry platform 104 may include computing device 108.

In some embodiments, the computing device 108 may be operated by a user such as a doctor, clinician, researcher, patient, or other individual. For example, the user may provide the cytometry data 106 as input to the computing device 108 (e.g., by uploading a file), and/or may provide user input specifying processing or other methods to be performed using the cytometry data 106.

In some embodiments, computing device 108 includes software configured to perform various functions with respect to the cytometry data 106. An example of computing device 108 including such software is described herein including at least with respect to FIG. 1D. In some embodiments, software on computing device 108 is configured to process the cytometry data to identify a respective cell or particle type 110 for each of the one or more events. Example techniques for processing the cytometry data 106 to determine cell and/or event types are described herein including at least with respect to FIGS. 1D, 2A-2B, and 3A-3D.

In some embodiments, technique 100 additionally includes processing the cytometry data and/or the identified cell or particle types using computing device 108 to determine one or more cell composition percentages for cell types in the biological sample. A cell composition percentage indicates the proportion of a particular cell type in the biological sample 102.

In some embodiments, a cell composition percentage for the biological sample 102 is compared to a cell composition percentage associated with a cohort to predict a diagnosis for the subject, to predict how the subject is likely to respond to a particular treatment, to select a treatment for the subject, and/or for any other suitable application, as aspects of the technology described herein are not limited in this respect. For example, if the cell composition percentage determined for the biological sample 102 for the subject is comparable to the cell composition percentage associated with a cohort of patients who responded well to a particular treatment, then this may indicate that the subject is likely to respond well to that treatment. Additionally, or alternatively, if the cell composition percentage determined for the biological sample 102 for the subject is comparable to the cell composition percentage associated with a cohort of patients diagnosed with a particular disease, then it may be likely that the subject has the disease.

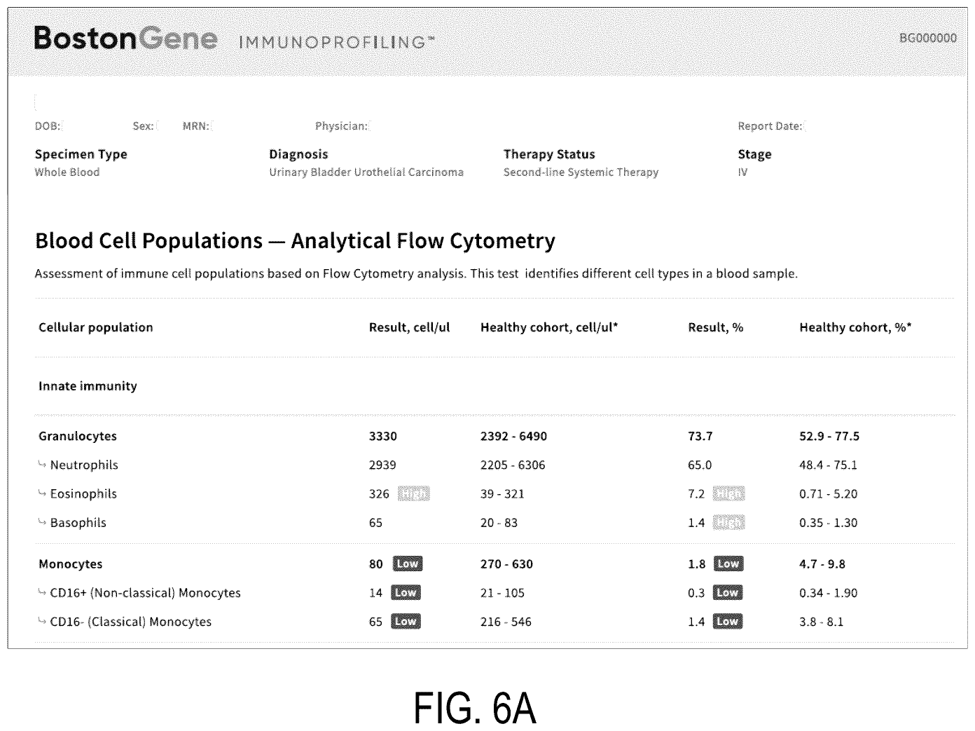

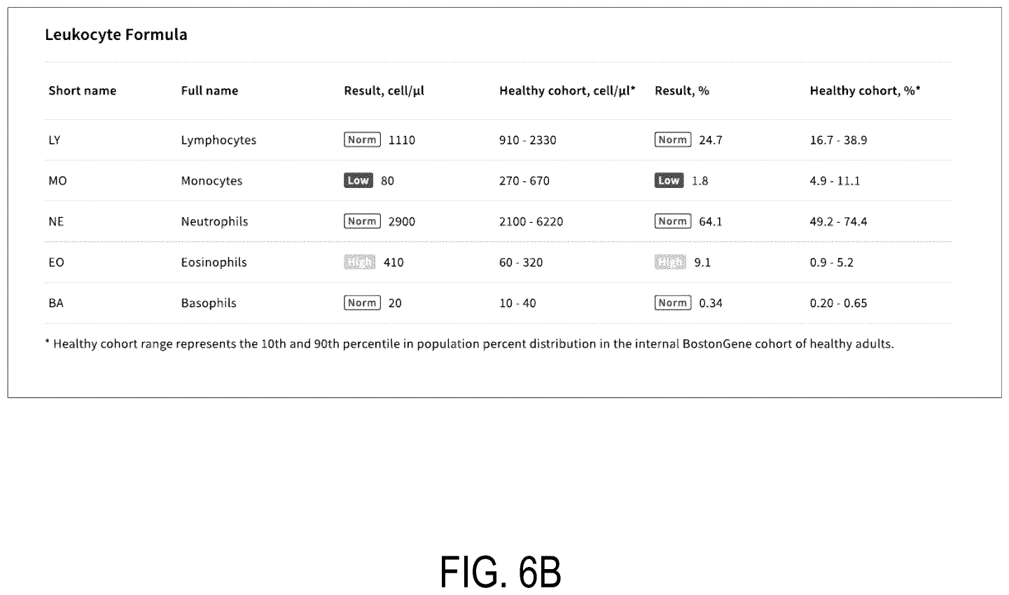

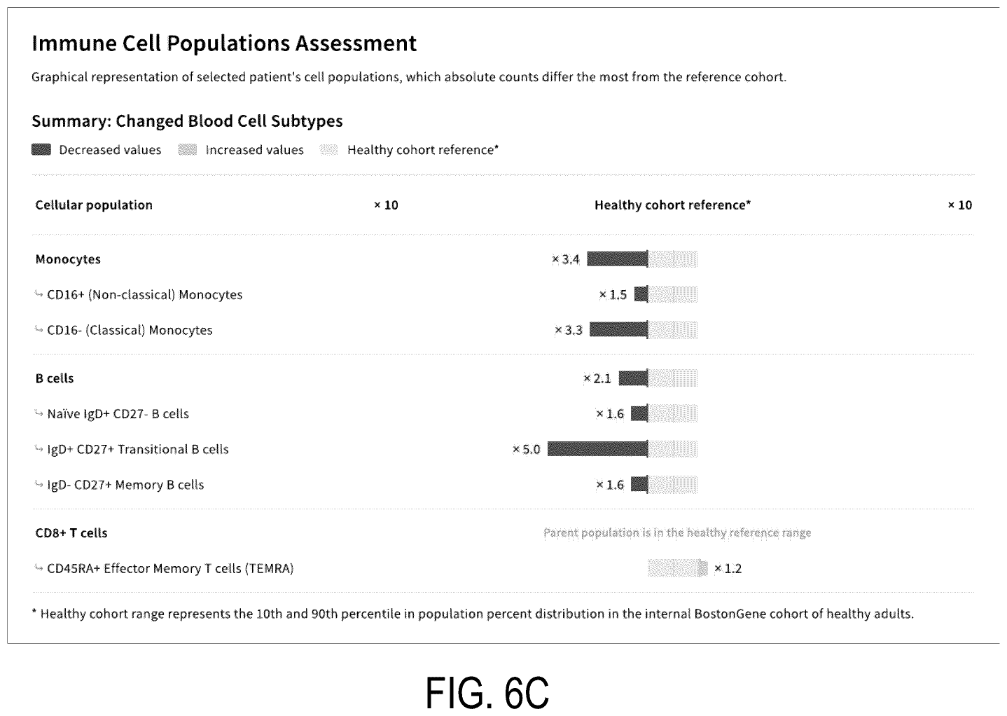

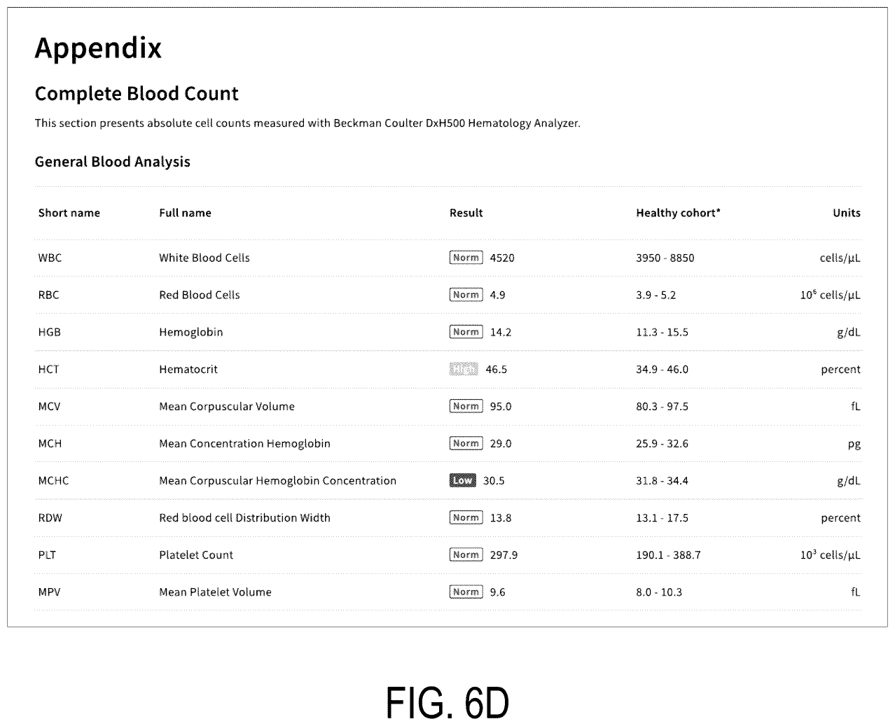

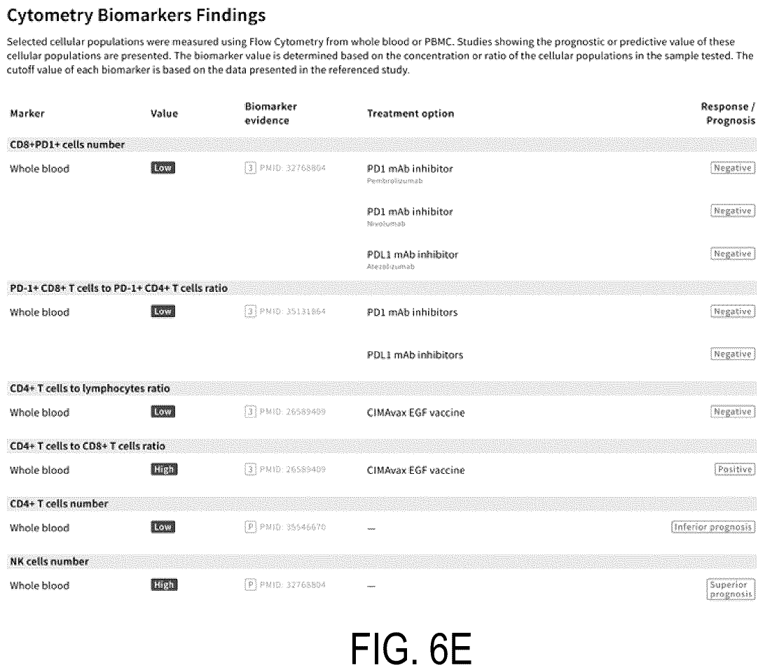

In some embodiments, technique 100 may include generating a report indicating the determined cell and/or particle types 110, cell composition percentages, predicted cohort, predicted treatments, predicted diagnoses, and/or any other suitable data resulting from technique 100. In some embodiments, the report may include graphics and/or text. In some embodiments, the report may be stored to memory or displayed via a user interface (e.g., a graphical user interface (GUI)) of a computing device (e.g., computing device 108). Example techniques for generating reports are described herein including at least with respect to FIGS. 1D and FIGS. 6A-6D.

As a nonlimiting example, technique 100 may be performed to determine cell composition percentages of different cell types in a subject suspected of having leukemia. A blood sample may be obtained by a physician and processed using a cytometry platform to obtain cytometry data. The cytometry data may be processed using a computing device to determine a respective cell or particle type for each event and to determine cell composition percentages for the determined cell and particle types. The cell composition percentages indicate the proportion of different cell populations (e.g., populations of different cell types) in the blood sample. For example, this could include determining the percentage of T cells in the blood sample. In some embodiments, the cell composition percentages are compared to those associated with different patient cohorts. For example, the estimate percentage of the subject's T cells may be compared to the percentage of T cells associated with a cohort of patients diagnosed with a particular disease. If the subject has a comparable T cell composition percentage, this may indicate that the subject is a member of the cohort (e.g., has the disease). A report may be generated that indicates the cohort identified for the subject and a visualization of the cell populations in the blood sample.

As shown in FIG. 1B, cytometry data 106 includes cytometry data for each of multiple events—event 1 to event N, in this example. The cytometry data 106 includes cytometry data 132-1 for the first event, cytometry data 132-2 for the second event, cytometry data 132-3 for the third event, cytometry data 132-2 for an Nth event, etc. In some embodiments, all of the events 1 to N correspond to cells. In some embodiments, a portion of the events 1 to N correspond to cells, while a portion of the events 1 to N correspond to particles (e.g., beads).

In some embodiments, the cytometry data 106 indicates one or more values for one or more markers used to obtain the cytometry data. A “marker” may include a protein found in a particular cell type or cell types. A “marker value” may be indicative of the expression of such a protein.

In some embodiments, the marker may be fluorescently labelled, and a flow cytometry platform may measure the intensity of the fluorescent light emitted from a particular cell as it is processed. Cells which express the marker at a greater expression level will result in higher marker values (e.g., a higher intensity measurement).

In some embodiments, different markers are labelled with differently-colored fluorescent proteins. This helps to distinguish between the expression of the different markers. In some embodiments, fluorescence intensity is measured for each color of fluorescence emitted from a cell, each of which may be associated with a particular respective marker. For example, if a cell emits green, red, and blue, fluorescent light, this may indicate that the cell expresses three different markers.

Additionally, or alternatively, in some embodiments, the marker may be labelled using a heavy metal ion tag, and a mass cytometry platform may measure the relative intensity (or abundance) of the heavy metal ion tag. The relative intensity of a tag quantifies the amount of the ion produced in relation to the amount of the most abundant ion. In some embodiments, relative intensity is measured for each heavy metal ion tag detected from a cell, each of which may be associated with a particular respective marker.

FIG. 1C shows example marker values included in the cytometry data 106 for example markers (e.g., CD3, CD62L, CD27, CD45+, and IgA+).

According to some embodiments, each of the example markers is labelled with a particular color fluorescent protein, and the corresponding marker value indicates the intensity of the fluorescence of that color emitted from the cell. For example, consider a marker CD3 that is labelled with a green fluorescent protein. The marker values for CD3 for the multiple cells represents the intensity of green fluorescence emitted from those cells and measured by a flow cytometry platform.

According to some embodiments, each of the example markers is labelled with a particular heavy metal ion tag, and the corresponding marker value indicates the intensity of the tag relative to the most abundant ion, as measured by a mass cytometry platform.

It should be appreciated that the markers shown in FIG. 1C are nonlimiting examples and any suitable marker or combination of markers may be used in conjunction with some aspects of the technology described herein.

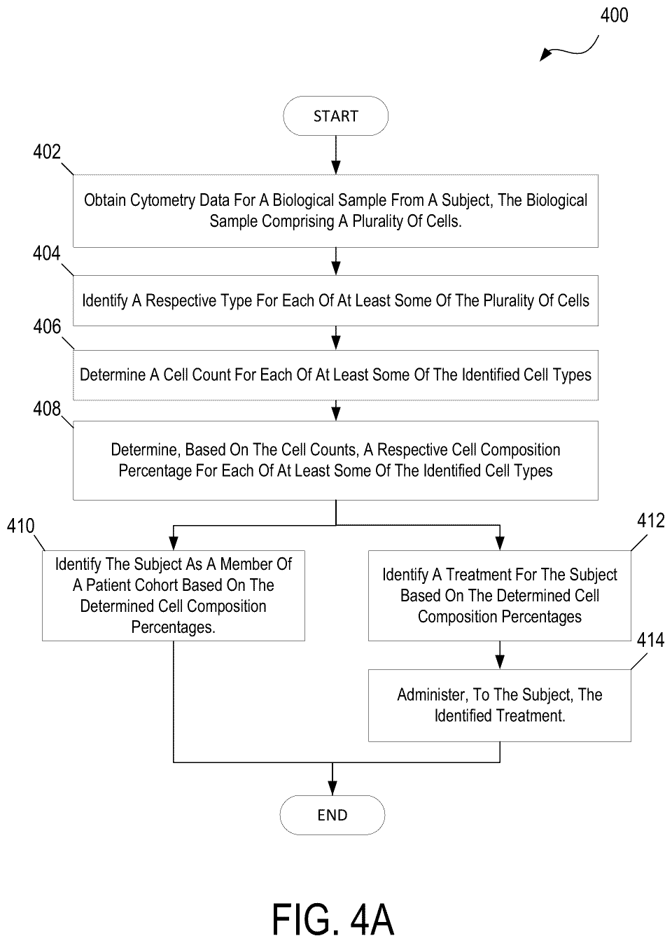

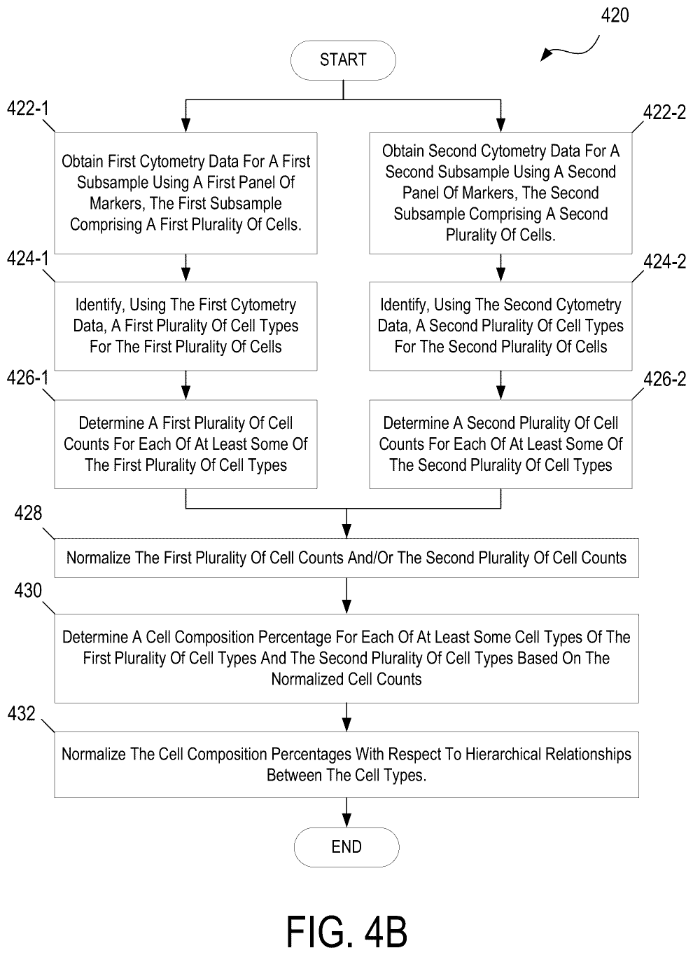

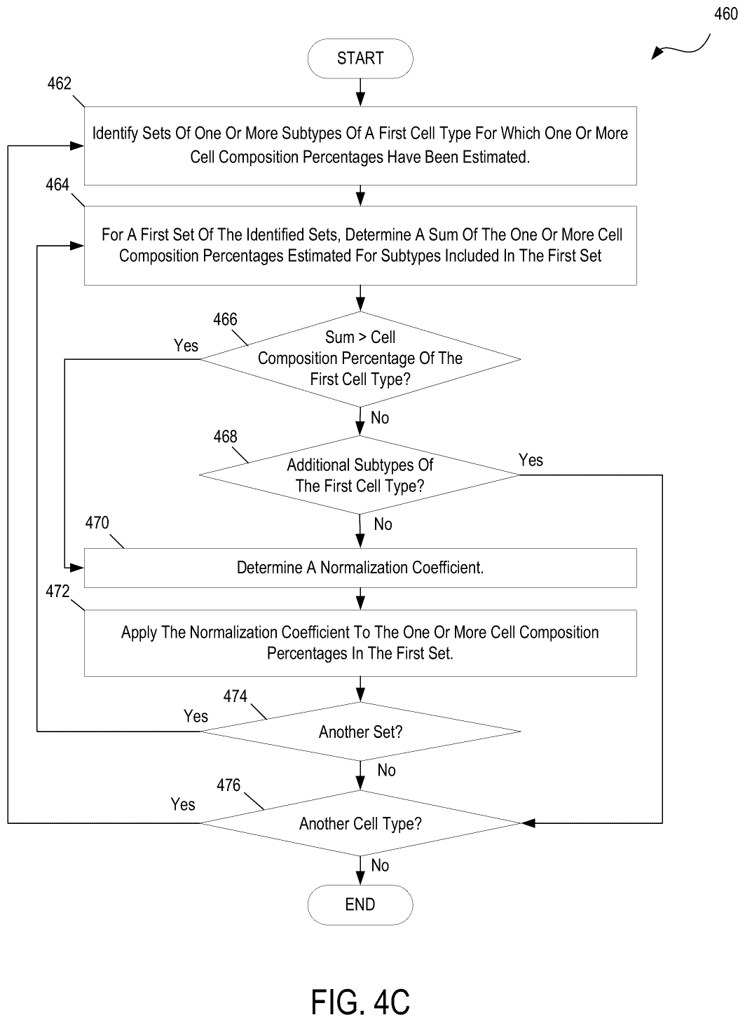

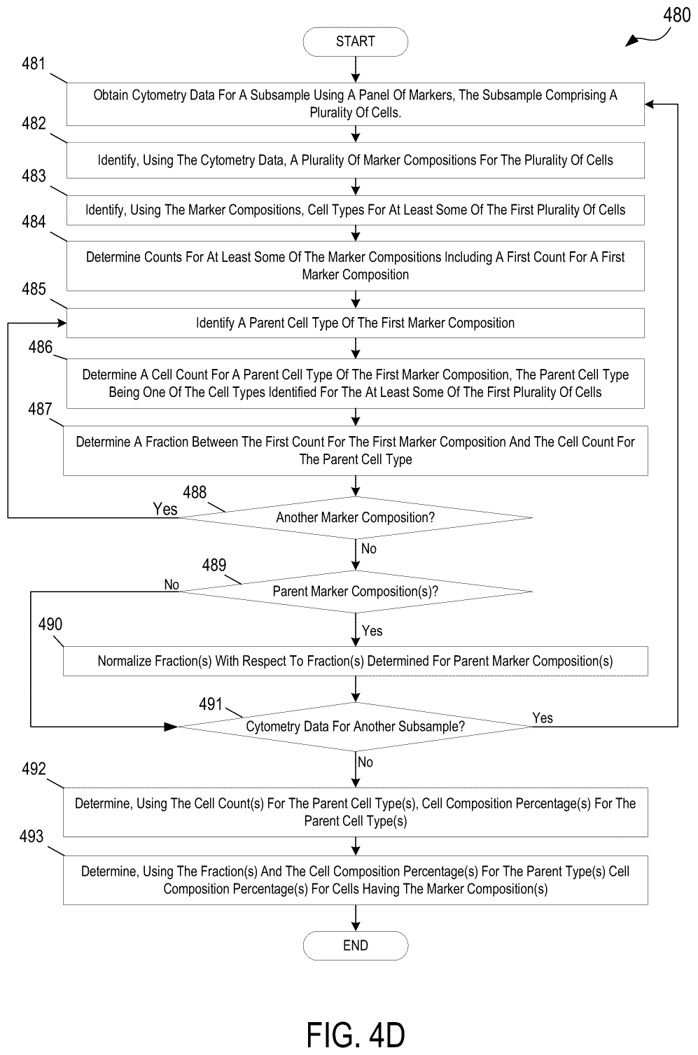

In some embodiments, computing device 108, shown in FIG. 1A, includes software 112 configured to perform various functions with respect to the cytometry data 106. In some embodiments, software 112 includes a plurality of modules. A module may include processor-executable instructions that, when executed by at least one computer hardware processor, cause the at least one computer hardware processor to perform the function(s) of the module. Such modules are sometimes referred to herein as “software modules.” each of which includes processor executable instructions configured to perform one or more processes, such as the processes described herein including at least with respect to FIGS. 2A-2B and FIGS. 4A-4C.

FIG. 1D is a block diagram of a system 150 including example computing device 108 and software 112, according to some embodiments of the technology described herein. Software 112 includes one or more software modules for processing cytometry data, such as an event type determination module 172, a cell composition percentage module 174, a cohort identification module 176, and a report generation module 178. In some embodiments, the software 112 additionally includes a user interface module 170, a cytometry platform interface module 162, and/or a data store interface module 160 for obtaining data (e.g., user input, cytometry data, one or more machine learning models). In some embodiments, data is obtained from cytometry platform 152, cytometry data store 154, and/or machine learning model data store 166. In some embodiments, the software 112 further includes a machine learning model training module 164 for training one or more machine learning models (e.g., stored in machine learning model data store 166.)

In some embodiments, data obtained from the cytometry data store 154 and/or the cytometry platform 152 and machine learning models obtained from the machine learning model data store 166 are used by the event type determination module 172 to determine types for one or more events. In some embodiments, the obtained data includes cytometry data for a biological sample from a subject. For example, the cytometry data may include cytometry measurements for each of multiple events in the biological sample, such as first cytometry measurements for a first event.

In some embodiments, the event type determination module 172 determines a respective type for each of at least some of the events in the biological sample using at least one trained machine learning model. For example, in some embodiments, this includes processing cytometry measurements corresponding to a particular event using one or more machine learning models to determine an event type for the event. For example, the machine learning model(s) may include a multiclass classifier trained to predict an event type from among multiple event types. Additionally, or alternatively, the machine learning model(s) may include one or more binary classifiers each trained to predict whether the event is of a particular event type. When the event type indicates that the particular event corresponds to a cell being processed with a cytometry platform, then the event type determination module 172, in some embodiments, processes the cytometry measurements corresponding to the particular event using a second machine learning model to determine a type of cell for the event. For example, the second machine learning model may include one or more multiclass classifiers trained to predict a type of cell for the event from among multiple cell types. Additionally, or alternatively, the second machine learning model may include one or more binary class classifiers. In some embodiments, the binary classifiers are each trained to predict whether the event corresponds to obtaining measurements for a cell of a particular type. In some embodiments, the binary classifiers are each trained to predict a degree of expression of a particular marker in a cell. For example, a binary classifier may be trained to predict whether a particular marker is expressed or not (e.g., whether expression of the marker exceeds a threshold.) In some embodiments, degrees of markers expression may be used to identify a type for a cell. In some embodiments, the event type determination module 172 processes the cytometry data according to the techniques described herein, including at least with respect to FIGS. 2A-C, to determine types for the events included in the biological sample.

In some embodiments, the event type determination module 172 obtains the cytometry data and/or the machine learning models via one or more interface modules. In some embodiments, the interface modules include cytometry platform interface module 162 and data store interface module 160. The cytometry platform interface module 162 may be configured to obtain (either pull or be provided) cytometry data from the cytometry platform 152. The data store interface module 160 may be configured to obtain (either pull or be provided) cytometry data and/or machine learning models from the cytometry data store 154 and/or the machine learning model data store 166, respectively. The data and/or machine learning models may be provided via a communication network (not shown), such as Internet or any other suitable network, as aspects of the technology described herein are not limited to any particular communication network.

In some embodiments, cytometry data store 154 includes any suitable data store, such as a flat file, a data store, a multi-file, or data storage of any suitable type, as aspects of the technology described herein are not limited to any particular type of data store. The cytometry data store 154 may be part of software 112 (not shown) or excluded from software 112, as shown in FIG. 1D.

In some embodiments, cytometry data store 154 stores cytometry data obtained from biological sample(s) of one or more subjects. In some embodiments, the cytometry data may be cytometry data from cytometry platform 152 and/or cytometry data obtained from one or more public data stores and/or studies. In some embodiments, a portion of the cytometry data may be processed with the event type determination module 172 to determine types for events associated with the cytometry data. In some embodiments, a portion of the cytometry may be used to train one or more machine learning models (e.g., with the machine learning model training module 164). In some embodiments, a portion of the cytometry data may include additional data (e.g., event types and/or cell composition percentages) and may be associated with one or more patients in a cohort. This portion of cytometry may be used, for example, by the cohort identification module 176 to identify a subject as a member of a cohort.

In some embodiments, machine learning model data store 166 includes any suitable data store, such as a flat file, a data store, a multi-file, or data storage of any suitable type, as aspects of the technology described herein are not limited to any particular type of data store. The machine learning model data store 166 may be part of software 112 (not shown) or excluded from software 112, as shown in FIG. 1D.

In some embodiments, machine learning model data store 166 stores one or more machine learning models. For example, the machine learning model data store 166 may store a machine learning model trained to predict an event type for an event. Additionally, or alternatively, the machine learning model data store 166 may store one or more machine learning models trained to predict a type for a cell. Additionally, or alternatively, the machine learning model data store 166 may store one or more machine learning models trained to predict a degree to which a marker is expressed in cell. In some embodiments, the machine learning model data store 166 stores relationships between at least some of the machine learning models. For example, the machine learning model data store 166 may store one or more sets of machine learning models. A set of machine learning models may include multiple machine learning models, the outputs of which may be used in combination to determine a cell type or event type. As another example, the machine learning model data store 166 may store hierarchical relationships between machine learning models. For example, a parent machine learning model may have one or more child machine learning models, one or more of which may be accessed based on an output of the parent machine learning model.

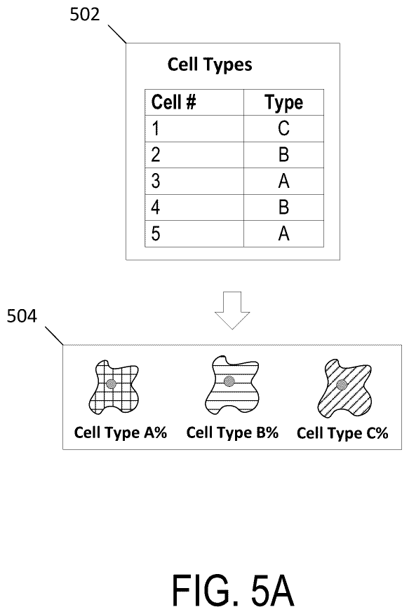

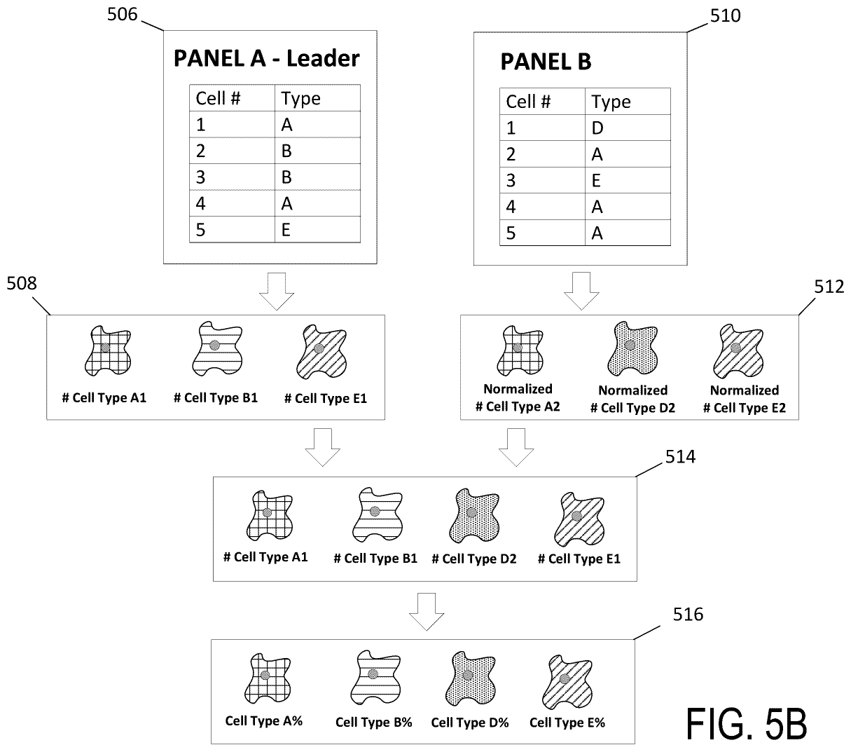

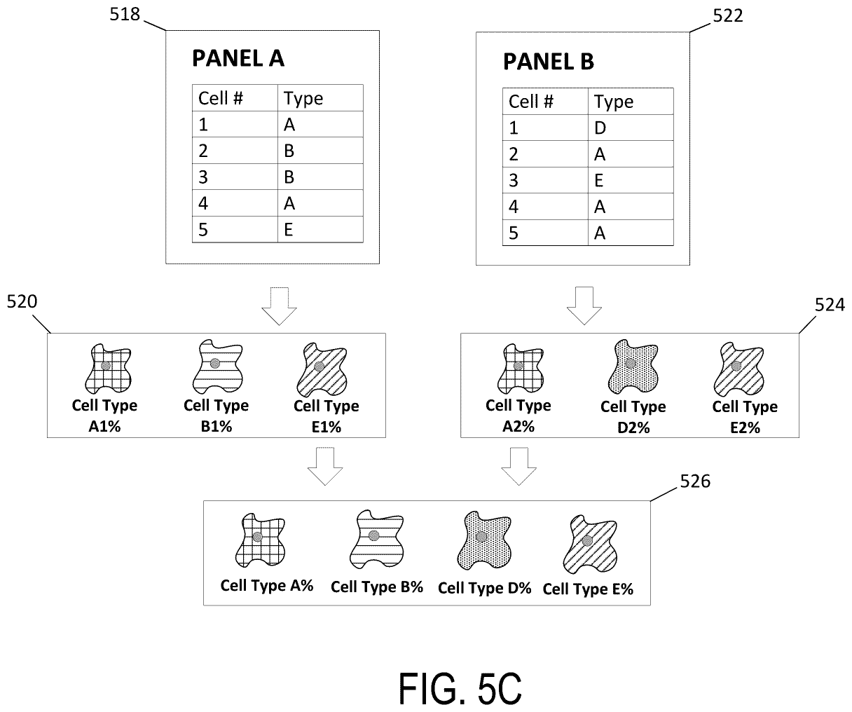

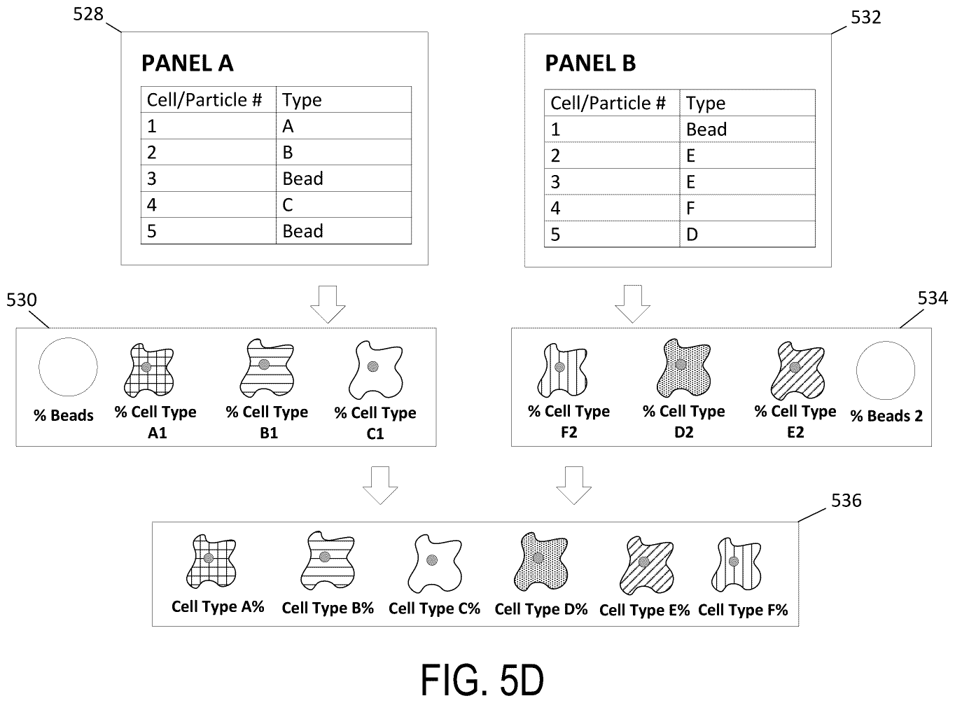

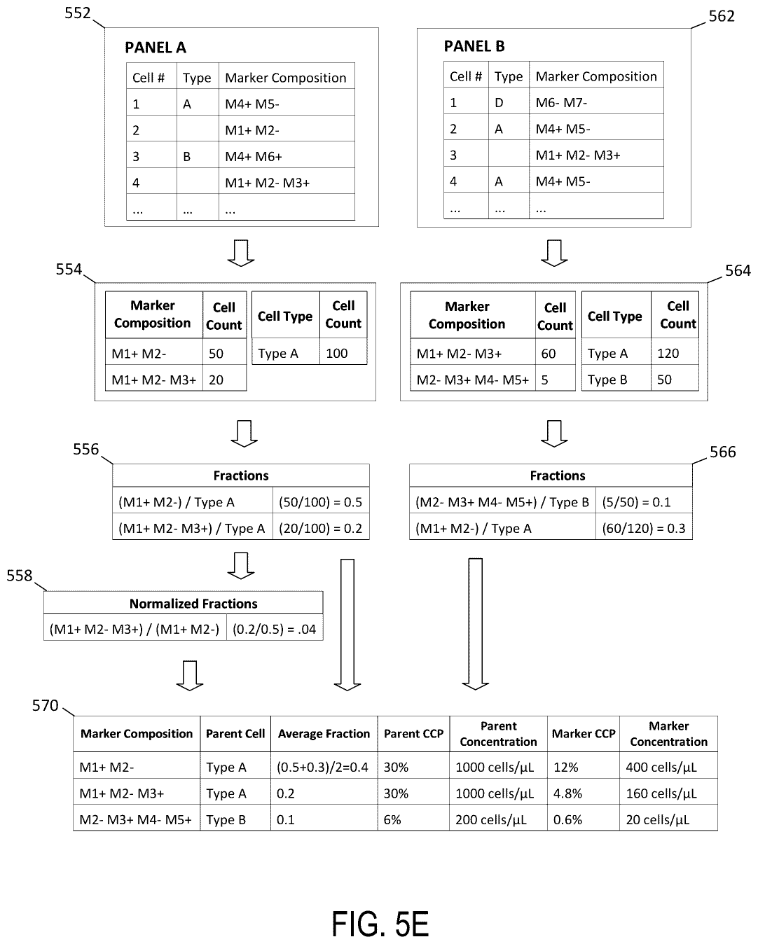

In some embodiments, cell composition percentage module 174 estimates cell composition percentages for cell populations of different types in a biological sample. In doing so, the cell composition percentage module 174 may use labelled cytometry data indicating the event types of events for which cytometry data has been obtained. For example, the labelled cytometry data may include cytometry data for a first event that is labelled with the type for the first event. In some embodiments, the labels may be determined by the event type determination module 172 and/or determined through alternative means. In some embodiments, the cell composition percentage module 174 estimates the cell composition percentages for cell types included in the biological sample. This includes, in some embodiments, estimating cell composition percentages for cell types included in subsamples of the biological sample. Example techniques for estimating cell composition percentages are described herein including at least with respect to FIGS. 4A-4D and 5A-5E.

In some embodiments, cohort identification module 176 identifies one or more patient cohorts to which the subject (e.g., from whom the biological sample was obtained) belongs. This may include comparing the cell composition percentages determined for the subject to those associated with one or more patient cohorts. In some embodiments, the cohort identification module 176 may obtain data associated with the one or more patient cohorts from the data store interface module 160. Additionally, or alternatively, the cohort identification module 176 may obtain input from user 168 via user interface module 170 indicating one or more cohorts (and their associated cell composition percentages) to which the cell composition percentages of the subject should be compared. Example techniques for identifying a subject as a member of a cohort are described herein including at least with respect to FIG. 4A.

In some embodiments, report generation module 178 processes results obtained from the event type determination module 172, the cell composition percentage module 174, and/or the cohort identification module 176 to generate one or more reports. For example, the one or more reports may indicate the event types included in the biological sample, the proportions of cell populations (e.g., cell composition percentages) in the biological sample, and/or one or more cohorts to which the subject belongs. In some embodiments, the one or more reports may additionally, or alternatively, indicate results from a hematology analyzer. Additionally, or alternatively, the one or more reports may indicate any other suitable information, such as, for example, a diagnosis for the subject, a suggested treatment, and/or relationships between cell populations. In some embodiments, the reports may include visualizations such as charts, graphs, tables, and/or any other suitable visualization for displaying the data. Example reports are described herein including at least with respect to FIGS. 6A-6D.

User interface 170 may be a graphical user interface (GUI), a text-based user interface, and/or any other suitable type of interface through which a user may provide input. For example, in some embodiments, the user interface may be a webpage or web application accessible through an Internet browser. In some embodiments, the user interface may be a GUI of an app executing on the user's mobile device. In some embodiments, the user interface may include a number of selectable elements through which a user may interact. For example, the user interface may include dropdown lists, checkboxes, text fields, or any other suitable element.

In some embodiments, machine learning model training module 164, referred to herein as training module 164, is configured to train the one or more machine learning models used to determine a type for an event, a type for a cell, and/or a degree to which a marker is expressed in a cell. In some embodiments, the training module 164 trains a machine learning model using a training set of cytometry data. For example, the training module 164 may obtain training data via data store interface module 160. In some embodiments, the training module 164 may provide trained machine learning models to the machine learning model data store 166 via data store interface module 160.

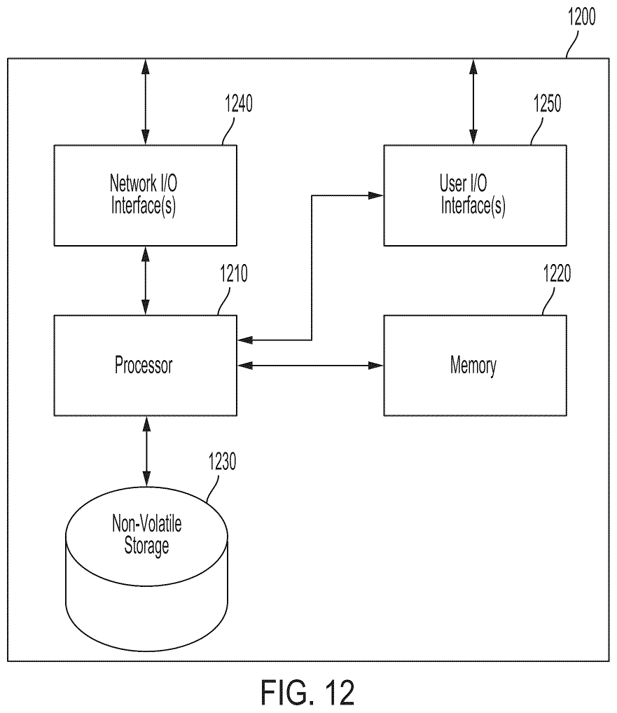

FIG. 2A is a flowchart of an illustrative process 200 for identifying types of cells present in a biological sample using multiple machine learning models, according to some embodiments of the technology described herein. Process 200 may be performed by a laptop computer, a desktop computer, one or more servers, in a cloud computing environment, computing device 108 as described herein with respect to FIG. 1A and FIG. 1D, computing device 1200 as described herein with respect to FIG. 12, or any other suitable computing device(s), as aspects of the technology described herein are not limited in this respect.

At act 202, cytometry data is obtained for a biological sample previously-obtained from a subject. In some embodiments, obtaining the cytometry data includes obtaining cytometry data using a cytometry platform, from a user, and/or from a data store storing such information. In some embodiments, the biological sample includes a plurality of objects including a plurality of cells. In some embodiments, the plurality of objects may additionally include particles such as debris and/or beads. For example, beads may be added to the biological sample in order to determine cell composition percentages of different cell types in the sample, as described herein including at least with respect to FIGS. 4B and 5D.

In some embodiments, the cytometry data includes cytometry measurements obtained during respective cytometry events. As described herein, a cytometry event corresponds to an object (e.g., a cell, debris, a bead, a doublet, or an undefined object) being measured by a cytometry platform (e.g., a flow cytometry platform or a mass cytometry platform). In some embodiments, the cytometry events include a subset of events corresponding to cells in the biological sample being measured by the cytometry platform. For example, the subset of events may include one, some, or all of the cytometry events. The number of cells measured using the cytometry platform may include any suitable number of cells, as aspects of the technology described herein are not limited in this respect. For example, the number of cells measured by the cytometry platform may include at least 5,000 cells, at least 10,000 cells, at least 20,000 cells, at least 50,000 cells, at least 100,000 cells, at least 500,000 cells, at least 600,000 cells, at least 900,000 cells, between 500 cells and 1 million cells, between 5,000 cells and 900,000 cells, or between 20,000 cells and 700,000 cells. In some embodiments, the measurements obtained during each event of the subset of events is included in the cytometry data obtained at act 202. For example, measurements obtained during a first event of the subsets of events may be included in the cytometry data, where the first event corresponds to a cell in the biological sample being measured by the cytometry platform. Examples of cytometry data are described herein including at least in the “Flow Cytometry” and “Mass Cytometry” sections.

In some embodiments, the cytometry data includes measurements for markers used to label the cells in the biological sample. In some embodiments, the measurements include fluorescent intensity values. For example, as described herein, a marker used to label the cells may be fluorescently labelled, and a flow cytometry platform may measure the intensity of the fluorescent light emitted from a particular cell as it is processed. Cells which express the marker at a greater expression level will result in higher marker values (e.g., a higher intensity measurement). Additionally, or alternatively, the measurements include relative intensities (or abundances) of heavy metal ion tags. As described herein, the markers used to label the cells may themselves be labeled using heavy metal ion tags. A mass cytometry platform may measure the relative intensity (or abundance) of the heavy metal ion tag. The relative intensity of a tag quantifies the amount of the ion produced in relation to the amount of the most abundant ion. In some embodiments, relative intensity is measured for each heavy metal ion tag detected from a cell, each of which may be associated with a particular respective marker.

In some embodiments, cells in the same biological sample may be split into two or more subgroups, termed “subsamples,” and labeled with different panels of markers. The different panels of markers may have some markers in common but differ with respect to at least one or more markers. A panel of markers may include any suitable number of markers such as, for example, at least 2 markers, at least 5 markers, at least 7 markers, at least 10 markers, at least 12 markers, at least 15 markers, at least 30 markers, between 2 and 40 markers, between 5 and 15 markers, or any other suitable number of markers. As a nonlimiting example, a panel of ten markers may be used to obtain cytometry measurements during a first event. The cytometry measurements obtained during the first event may include a measurement for each of at least some (e.g., some or all) of the ten markers). As described herein, including at least with respect to FIGS. 1B-1C, in some embodiments, a measurement for a marker may include a fluorescent intensity or relative intensity of a heavy metal ion tag measured for the marker.

In some embodiments, act 204 includes one or more optional steps for pre-processing the cytometry data. It should be appreciated that one, some, all, or none of the pre-processing techniques described herein may be used to pre-process the cytometry data, as embodiments of the technology described herein are not limited in this respect. One example of an optional pre-processing step is compensation. Compensation may be performed to remove the signal of a fluorochrome from detectors other than the one devoted to measuring that fluorochrome. Another example of an optional pre-processing step includes applying a transformation to the cytometry data. Since fluorescent intensity values included in the cytometry data follow an approximately log-normal distribution, transforming such data may bring the data closer to a normal distribution. Another example of an optional pre-processing step includes normalizing at least some of the measurements included in the cytometry data. Normalizing the cytometry data helps to reduce variations across cytometry data obtained using different cytometry platforms and/or obtained from different biological samples.

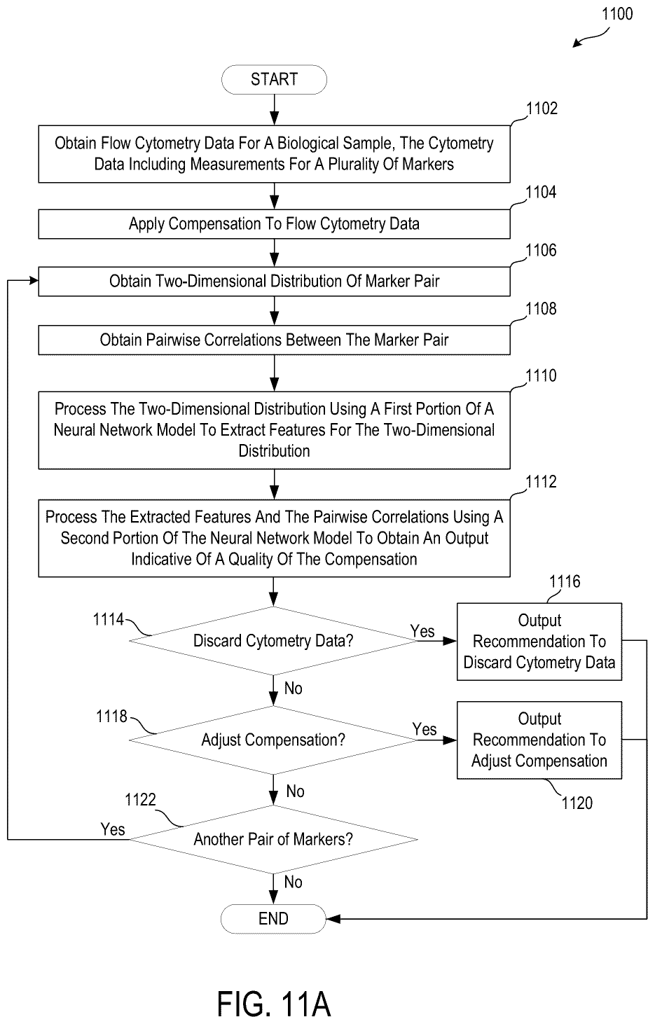

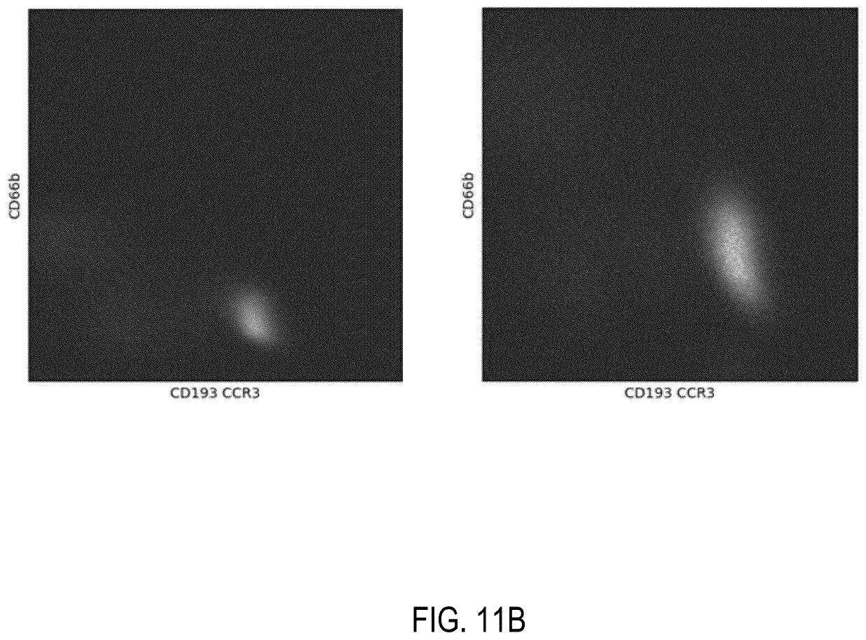

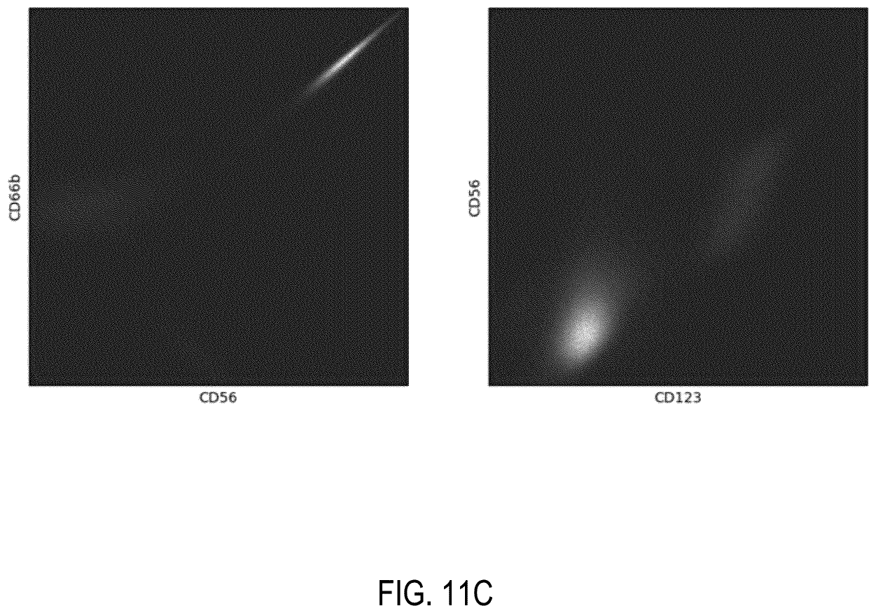

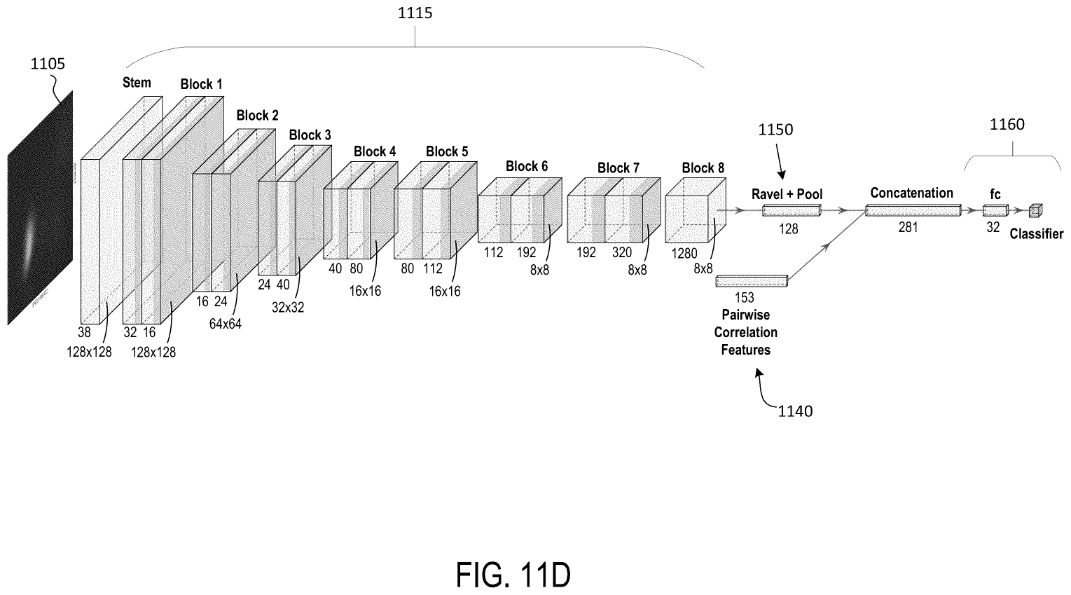

Accordingly, in some embodiments, at act 204, at least some of the cytometry data is compensated using any suitable compensation techniques, as embodiments of the technology described herein are not limited in this respect. In some embodiments, for example, a compensation matrix may be included in the metadata of a file (e.g., a flow cytometry standard (FCS) file) including the cytometry data. As another example, software, such as FlowJo™ (FlowJo™ Software. Ashland, OR: Beckton, Dickinson and Company; 2021), may be used to generate a compensation matrix. In some embodiments, such a compensation matrix may be applied to at least some cytometry measurements included in the cytometry data to obtain compensated cytometry measurements. Example techniques for compensating cytometry data and determining a quality of the applied compensation are described herein including at least with respect to FIGS. 11A-11D.

Additionally, or alternatively, at act 204, a transformation may be applied to at least some of the cytometry data. For example, this may include applying an inverse hyperbolic sine function to at least some of the measurements included in the cytometry data. For example, applying an inverse hyperbolic sine function may include applying Equation 1:

$$f ( x ) = arcsin h ( \frac{x}{c} ) \qquad ( Equation 1 )$$

where x is a marker value and c is a cofactor that influences the quality of clustering the cytometry data. According to some embodiments, c is determined experimentally and is selected to produce the highest quality of clustering. For example, the cofactor, c, may equal 190.

Additional, or alternative, examples of a transformation that may be applied to the cytometry data include the “Logicle” transformation described by Parks, D., et. al. (A new ‘Logicle’ display method a avoids deceptive effects of logarithmic scaling for low signals and compensated data. Cytometry Part A, Volume 69A, Issue 6, pp. 541-551), incorporated by reference herein in its entirety, and the “Hyperlog” transformation described by C. Bruce Bagwell (Hyperlog—a flexible log-like transform for negative, zero, and positive valued data. Cytometry Part A, Volume 64A, Issue 1, pp. 34-42), incorporated by reference herein in its entirety. In some embodiments, the transformation is applied to one or both of the compensated cytometry data and the uncompensated cytometry data.

Additionally, or alternatively, at act 204, at least some of the cytometry data may be normalized using any suitable normalization techniques, as embodiments of the technology described herein are not limited in this respect. In some embodiments, normalizing the cytometry data includes normalizing measurements for each of at least some markers. For example, the cytometry data may include multiple measurements for a particular marker. The multiple measurements may include a measurement for the marker obtained during each of at least some of the cytometry events. In some embodiments, normalizing the measurements for the marker may include normalizing the measurements with respect to a particular measurement included in the measurements for the marker. For example, this may include dividing measurements for the markers by a maximum or minimum measurement obtained for the marker. In some embodiments, normalizing the measurements for the marker may include normalizing the measurements with respect to a quantile value of measurements for the marker. For example, this may include dividing measurements obtained for the marker by the quantile value. A quantile value indicates a boundary value for which a certain percentage of cells is lower. The quantile value may include any suitable quantile value determined using any suitable techniques, as aspects of the technology are not limited in this respect. As a nonlimiting example, the quantile value may be at least 0.5 quantile, at least 0.6 quantile, at least 0.7 quantile, at least 0.75 quantile, at least 0.8 quantile, at least 0.9 quantile, at least 0.95 quantile, at least 0.98 quantile, at least 0.99 quantile, between 0.5 quantile and 1 quantile, or any other suitable quantile within the range of 0.5 to 1.

While various examples of techniques for processing cytometry data have been described, it should be appreciated that any other suitable data processing techniques may be used to process the cytometry data, as aspects of the technology described herein are not limited in this respect.

At act 206, types are identified for cells in the plurality of cells using multiple machine learning models to obtain a respective plurality of cell types. In some embodiments, the identification may be performed in two stages (e.g., a first stage corresponding to act 206-2 and a second stage corresponding to act 206-4), each involving different machine learning techniques. In the first stage, one or more machine learning models are used to identify which events correspond to cells and which correspond to one or more other objects (e.g., debris and beads). Then, in the second stage, for events corresponding to cells, one or more machine learning models are used to identify types for the cells.

At act 206-1, cytometry measurements corresponding to the particular event are obtained. This includes, in some embodiments, obtaining the cytometry measurements from the cytometry data obtained at act 202. For example, the cytometry measurements may include marker values (e.g., fluorescence intensity values, intensity of heavy metal ion tags, etc.) obtained during the particular event for markers in a panel of markers.

At act 206-2, an event type is identified for the particular event by processing the cytometry measurements obtained for the particular event using the machine learning model(s). In some embodiments, an event type indicates whether the particular event corresponds to a cell being measured by the cytometry platform, debris being measured by the cytometry platform, or a bead being measured by the cytometry platform. Additionally, or alternatively, the event type may indicate whether the particular event corresponds to multiple cells (e.g., a doublet) being measured by the cytometry platform. Additionally, or alternatively, the event type may indicate whether the particular event corresponds to an undefined object in the biological sample.

The machine learning model(s) may include any suitable machine learning model configured to predict an event type for an event. For example, the machine learning model(s) may include a multiclass classifier trained to predict an event type from among multiple event types. Additionally, or alternatively, the machine learning model(s) may include one or more binary classifiers, each trained to predict whether the event is of a particular event type. The machine learning model(s) may be trained to predict a probability that the event is of a particular event type. The machine learning model(s) may include any suitable machine learning model such as, for example, a decision tree classifier, a gradient boosted decision tree classifier, a neural network, or any other suitable type of machine learning model, as aspects of the technology described herein are not limited in this respect. The machine learning model(s) may be implemented using any suitable machine learning techniques including, for example, those described in the “Machine Learning” section.

In some embodiments, the machine learning model(s) are used to process cytometry measurements obtained for markers in a particular panel of markers. Accordingly, in some embodiments, a different machine learning model may be used to process cytometry measurements obtained for markers in different panels of markers. For example, the machine learning models may differ in that they are trained using different training data. For example, one machine learning model may be trained using cytometry measurements for markers in a first panel, while another machine learning model may be trained using cytometry measurements for markers in a second panel.

At act 206-3, when the identified event type indicates that the particular event corresponds to a cell being measured by the cytometry platform, one or more machine learning model(s) for identifying cell types may be selected. The selection may be done manually or automatically. In some embodiments, the selection may be made based on the specific type of panel of markers used to label cells in the biological sample. For example, when the panel may be used to identify fewer than a threshold number of cell types, then a single multi-class classifier may be selected at act 206-3. As another example, when the panel may be used to identify more than a threshold number of types, then a set of binary machine learning classifiers may be selected and subsequently used for identifying cell types. Examples of selecting machine learning model(s) based on panel types are described herein including in the “Example Cell Type Identification” section.

In some embodiments, machine learning model(s) selected at act 206-3 include any suitable number of machine learning models such as, for example, 1, at least 2, at least 5, at least 7, at least 10, at least 15, at least 20, at least 30, at least 40, at least 50, between 1 and 75, between 1 and 25, or any other suitable number of machine learning models, as embodiments of the technology described herein are not limited in this respect.

In some embodiments, each of the one or more machine learning models may be implemented using any suitable machine learning technique. For example, a machine learning model of the one or more machine learning model(s) may be a decision tree classifier, a gradient boosted decision tree classifier, a neural network, or any other suitable type of machine learning model, as aspects of the technology described herein are not limited in this respect. Additionally, or alternatively, the machine learning model(s) may be implemented using the machine learning techniques described in the “Machine Learning” section.

In some embodiments, the machine learning model(s) include at least one machine learning model trained to predict, based on cytometry measurements for a cell, a cell type for the cell. For example, the machine learning model(s) may include at least one multiclass classifier trained to predict a cell type for the cell from among multiple cell types. Additionally, or alternatively, in some embodiments, the machine learning model(s) may include one or more binary classifiers each trained to predict whether the cell is of a particular cell type. For example, the machine learning model(s) may be trained to predict a probability that the event is of a particular cell type.

In some embodiments, the machine learning model(s) include at least one machine learning model trained to predict, based on cytometry measurements for a cell, a degree to which a respective marker in the panel of markers is expressed in the cell. For example, the machine learning model(s) may include one or more binary classifiers, each of which corresponds to a marker in the panel of markers. Additionally, or alternatively, the machine learning model(s) may include one or more binary classifiers, each of which corresponds to an “artificial marker” that represents a combination of two or more markers in the panel of markers. In some embodiments, a binary classifier corresponding to a particular marker (e.g., a panel marker or an artificial marker) is trained to predict the degree to which the particular marker is expressed in the cell. Additionally, or alternatively, in some embodiments, the machine learning model(s) include a multiclass classifier trained to predict, for each of multiple markers included in the panel of markers and/or for each of the artificial markers, the degree to which the multiple markers are expressed in the cell.

In some embodiments, the output of a machine learning model trained to predict a degree to which a marker is expressed in a cell may include any suitable output. For example, in some embodiments, the output may indicate the expression level of at least one marker. In some embodiments, the output may indicate whether the expression level of at least one marker exceeds a threshold value. For example, the output may include a likelihood and/or a binary indication that the expression level exceeds a threshold value. The threshold may include any suitable threshold value such as, for example, a value of expression of a negative control. In some embodiments, the output may indicate whether at least one marker is expressed in the cell. For example, the output may indicate a likelihood (e.g., a probability) that the marker is expressed in the cell. In some embodiments, when the machine learning model is trained to predict a degree to which an artificial marker is expressed in a cell, the output of said machine learning model may be indicative of whether or not the markers represented by the artificial marker are expressed in a cell. For example, the output may include a binary output indicating whether or not the represented markers are expressed, a likelihood that represented markers are expressed, and/or an expression level of the combination of represented markers. Examples of artificial markers are described herein including at least with respect to the “Cell Type Identification” section.

At act 206-4, a type is identified for the cell by processing the cytometry data using the one or more machine learning model(s) selected at act 206-3. As described with respect to act 206-3, the selected machine learning model(s) may include (a) at least one machine learning model trained to predict a cell type for the cell or (b) at least one machine learning model trained to predict a degree to which a respective marker is expressed in the cell.

When the machine learning model(s) include the latter type of machine learning model(s), the cell type may be identified based on the predicted degree(s) to which respective marker(s) are expressed. For example, in some embodiments a particular cell type is associated with particular degrees of expression of particular markers. For example, CD45 may typically be expressed in neutrophils, while CD193 CCR3 may not typically be expressed in neutrophils. In this example, if the machine learning model(s) predict that CD45 (among other markers) is expressed in a cell and that CD193 CCR3 (among other markers) is not expressed in the cell, then neutrophil may be identified as the type for the cell. Tables 1B, 2C, 7B, 8C, and 10C list examples of cell types and degrees of marker expression with which the cell types are associated. In some embodiments, if the machine learning model(s) output a particular combination of degrees of marker expression that is not associated with any cell type, then the cell type associated with the closest combination of degrees of marker expression is identified for the cell.

At act 206-5, after identifying the cell type for the particular event, process 200 includes determining whether the subset of events includes another event. If the subset of events includes another event, one or more of acts 206-1, 206-2, 206-3, and 206-4 may be repeated for the event. For example, cytometry measurements corresponding to the next event may be used to determine an event type and/or a cell type for the event. If, at act 206-5, it is determined that the subset of events does not include another event, then process 200 ends.

It should be appreciated that process 200 may include one or more additional or alternative acts, which are not shown in FIG. 2A. For example, process 200 may include all of acts 202, 204, 206, 206-1, 206-2, 206-3, 206-4, and 206-5; only acts 202, 206, 206-1, 206-2, 206-3, and 206-4; only acts 202 and 206-1, 206-2, and 206-4; only acts 202, 204, 206, 206-1, 206-2, and 206-3, or any other suitable combination of acts. It should be appreciated that one or more of the acts may be performed in any order. For example, act 206-3 may be performed prior to act 206-2, prior to act 206, or prior to act 204.

In some embodiments, as described herein in more detail, the cell types identified as a result of process 200 are used to determine cell composition percentages of different types of cells in the biological sample. For example, determining a first cell composition percentage for a first type of cell may include determining a ratio between a number of cells in the identified as being of the first type and a total number of cells. Example techniques for determining cell composition percentages are described herein including at least with respect to FIGS. 4A-5E.

FIG. 2B is a flowchart depicting an illustrative process 250 for identifying types of cells present in a biological sample using a set of machine learning models each of which corresponds to a respective marker (e.g., marker in a panel of markers or artificial marker), according to some embodiments of the technology described herein. Process 250 may be performed by a laptop computer, a desktop computer, one or more servers, in a cloud computing environment, computing device 108 as described herein with respect to FIG. 1D, computing device 1200 as described herein with respect to FIG. 12, or any other suitable computing device(s), as aspects of the technology described herein are not limited in this respect.

Process 250 begins at act 252, where cytometry data is obtained for a biological sample previously-obtained from a subject. In some embodiments, obtaining the cytometry data includes obtaining cytometry data using a cytometry platform, from a user, and/or from a data store storing such information. In some embodiments, the biological sample includes a plurality of objects including a plurality of cells. In some embodiments, the plurality of objects may additionally include particles such as debris and/or beads. For example, beads may be added to the biological sample in order to determine cell composition percentages of different cell types in the sample, as described herein including at least with respect to FIGS. 4B and 5D.

In some embodiments, the cytometry data includes cytometry measurements (e.g., flow cytometry measurements, mass cytometry measurements, etc.) obtained for cells in the plurality of cells using a cytometry platform (e.g., a flow cytometry platform, a mass cytometry platform, etc.). In some embodiments, the cytometry measurements of a particular cell include measurements for markers included in a panel of markers. The panel of markers may include any suitable number of markers such as, for example, at least 2 markers, at least 5 markers, at least 7 markers, at least 10 markers, at least 12 markers, at least 15 markers, at least 30 markers, between 2 and 40 markers, between 5 and 15 markers, or any other suitable number of markers. As a nonlimiting example, a panel of ten markers may be used to obtain cytometry measurements for a first cell. The cytometry measurements obtained for the first cell may include a measurement for each of at least some (e.g., some or all) of the ten markers. As described herein, including at least with respect to FIGS. 1B-1C, in some embodiments, a measurement for a marker may include a fluorescent intensity value measured for the marker.

In some embodiments, cells in the same biological sample may be split into two or more subgroups, termed “subsamples,” and fluorescently labeled with different panels of markers. The different panels of markers may have some markers in common but differ in at least one or more markers. A panel of markers may include any suitable number of markers such as, for example, at least 2 markers, at least 5 markers, at least 7 markers, at least 10 markers, at least 12 markers, at least 15 markers, at least 30 markers, between 2 and 40 markers, between 5 and 15 markers, or any other suitable number of markers. As a nonlimiting example, a panel of ten markers may be used to obtain cytometry measurements during a first event. The cytometry measurements obtained during the first event may include a measurement for each of at least some (e.g., some or all) of the ten markers). As described herein, including at least with respect to FIGS. 1B-1C, in some embodiments, a measurement for a marker may include a fluorescent intensity value measured for the marker.

At act 254, the cytometry data is processed using one or more processing techniques. For example, the processor may process the cytometry data using any of the processing techniques described with respect to act 204 of process 200 in FIG. 2A.

At act 256, cell types are identified for at least a subset of the plurality of cells using a set of machine learning models. The subset of cells may include any suitable number of cells, as aspects of the technology described herein are not limited in this respect. As nonlimiting examples, the subset of cells may include at least 5,000 cells, at least 10,000 cells, at least 20,000 cells, at least 50,000 cells, at least 100,000 cells, at least 500,000 cells, at least 600,000 cells, at least 900,000 cells, between 500 cells and 1 million cells, between 5,000 cells and 900,000 cells, or between 20,000 cells and 700,000 cells.

In some embodiments, the set of machine learning models includes any suitable number of machine learning models. For example, the set of machine learning models may include 1, 2, 5, 8, 10, 12, 15, 20, 25, 30, 40, 50, between 1 and 50, between 2 and 25, or any other suitable number of machine learning models, as aspects of the technology are not limited in this respect.

In some embodiments, the set of machine learning models corresponds to a particular panel of markers. For example, the set of machine learning models may include a first machine learning model corresponding to a first marker in the panel of markers (e.g., trained to predict a degree of expression of the first marker) and a second machine learning model corresponding to a second marker in the panel of markers (e.g., trained to predict a degree of expression of the second marker). Additionally, or alternatively, the set of machine learning models may include a machine learning model corresponding to an “artificial marker.” An artificial marker may represent two or more markers in the panel of markers. Accordingly, a machine learning model corresponding to an artificial marker may be trained to predict a degree of expression of the particular combination of two or more markers. Example panels of markers are described herein including at least in the “Cell Type Identification Example” section.

A machine learning model in the set of machine learning models may include any suitable machine learning model such as, for example, a decision tree classifier, a gradient boosted decision tree classifier, a neural network, or any other suitable type of machine learning model, as aspects of the technology described herein are not limited in this respect. In some embodiments, the machine learning model is a binary classifier corresponding to a marker in the panel of markers. In some embodiments, a binary classifier corresponding to a particular marker is trained to predict the degree to which the particular marker is expressed in a cell. Additionally, or alternatively, in some embodiments, the machine learning model is a multiclass classifier trained to predict, for each of multiple markers included in the panel of markers, the degree to which the multiple markers are expressed in the cell. The machine learning model may be implemented using any of the machine learning techniques described in the “Machine Learning” section.

In some embodiments, identifying the types for at least the subset of the plurality of cells includes using multiple sets of machine learning models if the cytometry measurements were obtained using more than one panel of markers. Consider, for example, a first cell for which first cytometry measurements were obtained using a first panel of markers and a second cell for which second cytometry measurements were obtained using a second panel of markers. In this example, a first set of machine learning models may be used to predict a degree of expression in the first cell of each of at least some of the markers in the first panel and a second set of machine learning models may be used to predict a degree of expression in the second cell of each of at least some of the markers in the second panel. Examples of panels corresponding to sets of machine learning models are listed in Tables 1A-10C.

At act 256-1, first cytometry measurements of a first cell in the subset of the plurality of cells are obtained from the cytometry data. For example, the first cytometry measurements may include cytometry measurements obtained by the cytometry platform using a panel of markers including a first marker and a second marker. The first cytometry measurements may include marker values (e.g., fluorescence intensity values, intensity of heavy metal ion tags, etc.) obtained for markers in a panel of markers.

At act 256-2, the first cytometry measurements are processed using the first machine learning model in the set of machine learning models to obtain a first output indicating a degree to which the first marker in the panel of markers is expressed in the first cell. In some embodiments, the first output indicates the degree to which the first marker is expressed in the first cell includes any suitable output. For example, the first output may include any of the suitable outputs described herein including at least with respect to act 206-3 of process 200 in FIG. 2A.

At act 256-3, the first cytometry measurements are processed using the second machine learning model in the set of machine learning models to obtain a second output indicating a degree to which the second marker in the panel of markers is expressed in the first cell. In some embodiments, the second output indicates the degree to which the second marker is expressed in the first cell includes any suitable output. For example, the second output may include any of the suitable outputs described herein including at least with respect to act 206-3 of process 200 in FIG. 2A.

At act 256-4, a cell type is identified for the first cell using the first output indicating the degree to which the first marker is expressed in the first cell and the second output indicating the degree to which the second marker is expressed in the first cell. In some embodiments, this includes determining whether the first marker and/or the second marker is expressed in the first cell. For example, this may include determining whether the outputs of the first and second machine learning models indicate the presence or absence of the first and second markers, respectively. In some embodiments, the cell type may be identified based on the combination of markers that are deemed to be expressed (or not expressed). For example, the cell type may be identified using Tables 1B, 2C, 7B, 8C, and 10C. Tables 1B, 2C, 7B, 8C, and 10C list examples of cell types and degrees of marker expression with which the cell types are associated. A “+” symbol next to a marker in Tables 1B, 2C, 7B, 8C, and 10C indicates the presence of the marker in the cell type, while a “−” symbol next to a marker in Tables 1B, 2C, 7B, 8C, and 10C indicates absence of the marker.

At act 256-5, after identifying the cell type for the first cell, process 250 includes determining whether the subset of cells includes another cell. If the subset of cells includes another cell, one or more of acts 256-1, 256-2, 256-3, and 256-4 may be repeated for the next cell. For example, cytometry measurements corresponding to the next cell may be used to determine a cell type for the cell. If, at act 256-5, it is determined that the subset of events does not include another event, then process 250 ends.

It should be appreciated that process 250 may include one or more additional or alternative acts, which are not shown in FIG. 2B. For example, process 250 may include all of acts 252, 254, 256, 256-1, 256-2, 256-3, 256-4, and 256-5; only acts 252, 256, 256-1, 256-2, 256-3, and 256-4; only acts 252 and 256-1, 256-2, and 256-4; only acts 252, 254, 256, 256-1, 256-2, and 256-3, or any other suitable combination of acts.

In some embodiments, as described herein in more detail, the cell types identified as a result of process 250 are used to determine cell composition percentages of different types of cells in the biological sample. For example, determining a first cell composition percentage for a first type of cell may include determining a ratio between a number of cells in the identified as being of the first type and a total number of cells. Example techniques for determining cell composition percentages are described herein including at least with respect to FIGS. 4A-5E.

FIG. 3A is a diagram of an illustrative technique for identifying types of cells present in a biological sample using multiple machine learning models, according to some embodiments of the technology described herein. The illustrative technique is an example implementation of process 200 in FIG. 2A.

In some embodiments, the multiple machine learning models are used to process cytometry data 301. The cytometry data 301 may include cytometry measurements obtained during a cytometry event during which an object (e.g., a cell, debris, a bead, etc.) was measured using cytometry. In some embodiments, the cytometry measurements include a measurement for each of multiple markers in a panel of markers. For example, such a measurement may include a fluorescent intensity value for each marker.

As shown in FIG. 3A, the cytometry data 301 is processed using event type machine learning model 302 to predict an event type 303 for an event. The event type machine learning model 302 may be an implementation of the machine learning model(s) used to predict event types, described with respect to act 206-2. The model 302 may be a multi-class classifier and may be implemented as a gradient boosted decision tree classifier, for example. The model 302 may be used to assign a type to each event. For example, the model 302 may be used to assign a type from among the types: cell, debris, bead, and undefined.

At act 304, when the event type 303 indicates that the particular event is undefined, the type undefined 304-1 is assigned to the event. If the event type 303 indicates that the particular event corresponds to debris being measured by the cytometry platform, then the type debris 304-2 is assigned to the event.

At act 304, when the event type indicates that the particular event corresponds to a bead being measured by the cytometry platform, then bead type machine learning model(s) 304-3 may be used to process cytometry data 301. In some embodiments, a bead type machine learning model is trained to distinguish between beads of a first type (“Beads-1”) and beads of a second type (“Beads-2). In some embodiments, the different bead types indicate whether a single bead (“Beads-1”), or whether two, aggregated beads (“Beads-2”) were measured by the cytometry platform. Identifying aggregated beads may be useful for accurately estimating the number of beads included in the biological sample. In some embodiments, the bead type machine learning model(s) may be implemented according to any suitable machine learning techniques described herein including at least in the “Machine Learning” section. For example, the bead type machine learning model(s) may be a multi-class classifier implemented as a gradient boosted decision tree classifier.

At act 304, when the event type indicates that the particular event corresponds to a cell, a machine learning model(s) is selected, at act 305, from among cell type machine learning model(s) 305-1 and marker machine learning model(s) 305-2. In some embodiments, the machine learning model(s) are selected based on the panel of markers used to obtain cytometry data 301.

If the cell type machine learning model(s) 305-1 are selected at act 305, then the cytometry data 301 is processed using the cell type machine learning model(s) 305-1 to obtain an output indicating a type 306 for the cell. The cell type machine learning model(s) are an example implementation of the one or more machine learning model(s) trained to predict a cell type for the cell, described herein including at least with respect to act 206-3 of process 200 in FIG. 2A.

If the marker machine learning model(s) 305-2 are selected at act 305, then the cytometry data 301 is processed using the marker machine learning model(s) 305-2 to obtain one or more outputs 307 indicating the degree to which one or more markers are expressed in the cell. The marker machine learning model(s) are an example implementation of the at least one machine learning model trained to predict a degree to which a respective marker in a panel of markers is expressed in the cell, as described herein including at least with respect to act 206-3 of process 200 in FIG. 2A.

In some embodiments, the outputs 307 may be used to identify a cell type 308 for the cell. For example, as described herein, particular degrees of marker expression may be associated with particular cell types.

FIG. 3B is a diagram of an illustrative technique for identifying an event type 303 for an event using one or more machine learning models, according to some embodiments of the technology described herein.

The event type machine learning model(s) 302 may be used to predict, for each of multiple different event types, the probability that the event is of a particular type. For example, as shown in FIG. 3B, the event type machine learning model(s) 302 may be trained to predict a probability 311-1 that the event corresponds to a cell being measured by the cytometry platform, a probability 311-2 that the event corresponds to a bead being measured by the cytometry platform, and a probability 311-3 that the event corresponds to debris being measured by the cytometry platform. In some embodiments, the event type machine learning model(s) 302 may also be trained to predict probability 311-N that the event corresponds to an object type N, which may include any other suitable object type, as aspects of the technology described herein are not limited in this respect.

In some embodiments, the event type machine learning model(s) 302 may be implemented using any suitable type of machine learning model(s). For example, the event type machine learning model(s) 302 may be implemented using a single multi-class classifier or multiple binary classifiers each trained to predict the probability that the event is of the particular type.

In some embodiments, as indicated by combination logic 312, the technique 310 may predict an event type 303 based on probabilities 311-1, 311-2, 311-3, and 311-N. For example, in some embodiments, the type associated with highest probability may be selected. In some embodiments, the type may only be identified for the event if the highest probability exceeds a threshold. For example, if a probability of the particular type exceeds at least 0.5, at least 0.6, at least 0.75, at least 0.8, at least 0.85, at least 0.9, at least 0.95, at least 0.98, at least 0.99, between 0.5 and 1, or any other suitable threshold between 0.5 and 1, then the event may be identified as being of the particular type. If none of the probabilities exceed the threshold, then the type for the event may be identified as an undefined type.

FIG. 3C is a diagram of an illustrative technique for identifying a type 306 for a cell using one or more machine learning models, according to some embodiments of the technology described herein.

The cell type machine learning model(s) 312 may be used to predict, for each of multiple different cell types (e.g., N cell types), the probability that a cell is of a particular cell type. For example, as shown in FIG. 3C, the cell type machine learning model(s) 306 may be trained to predict a probability 321-1 that the cell is of a first type, a probability 321-2 that the cell is of a second type, the probability 321-3 that the cell is of the third type, and/or any probabilities that the cell is of any other suitable number of cell types, such as the probability 321-N that the cell is of the Nth type.

In some embodiments, the cell type machine learning model(s) 312 may be implemented using any suitable type of machine learning models. For example, the cell type machine learning model(s) 312 may be implemented using a single multi-class classifier or multiple binary classifiers each trained to predict the probability that the cell is of the particular type.

In some embodiments, as indicated by combination logic 322, the technique 320 may predict a cell type 306 based on probabilities 321-1, 321-2, 321-3, and 321-N. For example, in some embodiments, the type associated with highest probability may be selected. In some embodiments, the type may only be identified for the cell if the highest probability exceeds a threshold. For example, if a probability of a particular type exceeds at least 0.5, at least 0.6, at least 0.75, at least 0.8, at least 0.85, at least 0.9, at least 0.95, at least 0.98, at least 0.99, between 0.5 and 1, or any other suitable threshold between 0.5 and 1, then the cell is identified as being of the particular type. If none of the probabilities exceed the threshold, then the type for the cell may be identified as an undefined type.

FIG. 3D is a diagram of an illustrative technique for identifying a type for a cell using a set of machine learning models each of which corresponds to a marker in a panel of markers, according to some embodiments of the technology described herein.

As shown, the marker machine learning model(s) 305-2 include machine learning model 331-1, machine learning model 331-2, and any suitable number of other machine learning models, including machine learning model 331-N. In some embodiments, the machine learning model 331-1 is used to process cytometry data 301 to obtain an output 333-1 indicating a degree to which marker A is expressed by the cell. For example, output 333-1 may indicate whether marker A is expressed (A+) or not expressed (A−) by the cell. In some embodiments, the machine learning model 331-2 is used to process cytometry data 301 to obtain an output 333-2 indicating a degree to which marker B is expressed. For example, output 333-2 may indicate whether marker B is expressed (B+) or not expressed (B−) by the cell. In some embodiments, the machine learning model 331-N is used to process cytometry data 301 to obtain an output 333-N indicating a degree to which marker N is expressed. For example, output 333-N may indicate whether marker N is expressed (N+) or not expressed (N−) by the cell.

In some embodiments, as indicated by combination logic 332, the technique 330 may predict a cell type 308 based on outputs 333-1, 333-2, and 333-N. For example, as described herein, particular degrees of marker expression may be associated with particular cell type. Examples of identifying a cell type based on marker expression are described in the “Cell Type Identification” section.

### Cell Type Identification Example

As described herein including at least with respect to FIGS. 2A-3D, one or more machine learning models may be used to identify a type for a cell in a biological sample. For example, the machine learning model(s) may include machine learning model(s) trained to predict a cell type for the cell, or machine learning model(s) trained to predict the degree to which particular markers are expressed in the cell.

Regardless of whether the machine learning model(s) are trained to predict cell types or trained to predict the degree to which markers are expressed, the machine learning model(s) may be selected from among a single multiclass classifier or multiple binary classifiers. In some embodiments, the selection may be based on the type of panel used for labeling cells in the biological sample. For example, when the panel can be used to identify fewer than a threshold number of types, then a single multi-class classifier may be selected. As another example, when the panel can be used to identify more than a threshold number of types, then a set of binary machine learning classifiers may be selected act subsequently used for identifying cell types.

Example panels are listed in Tables 1A-10C. As shown in Tables 1A-10C, each panel includes a list of example markers. The markers may be labeled with fluorophores for detection during flow cytometry.

Tables 1A-10C also list one or more example machine learning models that may be used to identify a cell type for cells in a sample labeled using the panel of markers. For example, as shown in Table 1A, multiple binary classifiers may be used to identify cell types for cells labeled with markers in panel 1. In the example of Table 1A, each binary classifier is trained to predict a degree to which a particular marker is expressed. In some embodiments, the degree to which the particular markers are expressed may be used to identify a cell type for the cell. Table 1B, lists example cell types that are associated with the expression of markers predicted using the binary classifiers. For example, the absence of CD8 (CD8−) may be used to identify CD8−T as the cell type for a cell for which CD8− is predicted.

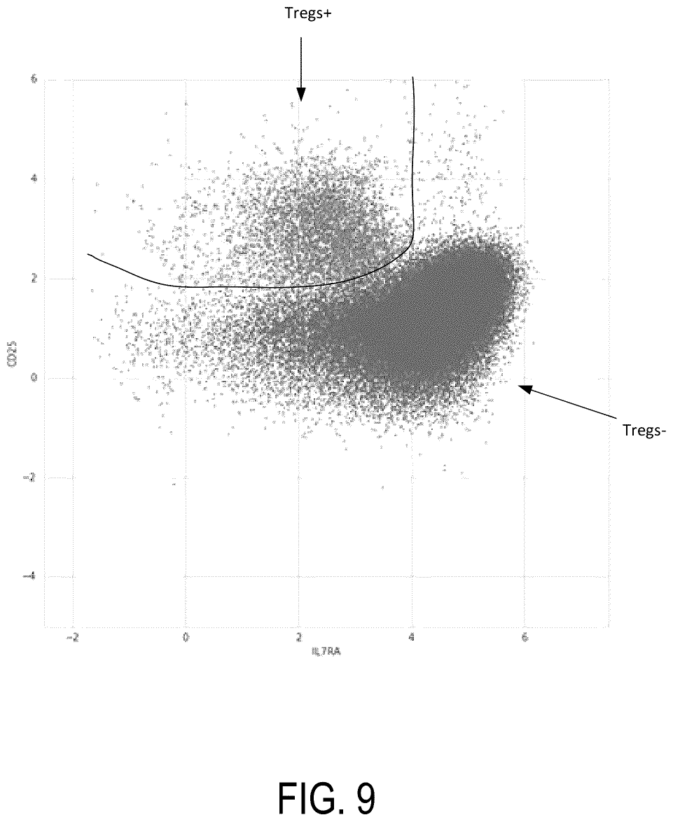

As described herein, in some embodiments, one or more of the binary classifiers may be trained to predict a degree to which a particular combination of markers (e.g., an artificial marker) is expressed. In some cases, the expression of a combination of two or more markers possesses biological significance, while the expression of the individual markers, on their own, lacks biological significance. As a result, it can be challenging to obtain an amount of training data for a particular individual marker that is sufficient to train a machine learning to predict the degree to which that particular marker is expressed. Therefore, to improve the efficiency of model training, in some embodiments, a machine learning model is trained to predict the degree to which a marker combination (e.g., an artificial marker) is expressed. For example, the regulatory T cell (Tregs) population is identified by the combination of the expression of two markers: CD25+ and ILR7A−. As shown in FIG. 9, annotation can be used to delineate the Tregs population (e.g., having marker expression: CD25+ ILR7A−) from the non-Tregs populations (e.g., having marker expression: CD25− ILR7A−, CD25−ILR7a+, and CD25+ILR7A+). The cell populations are not further subdivided into CD25 expression and ILR7A expression because on their own, in this example, those markers lack biological significance. Rather, the combined expression of CD25 and ILR7A is sufficient to divide the cell populations into: Tregs and non-Tregs. Accordingly, in some embodiments, a machine learning model (e.g., a binary machine learning model) can be trained to predict the “expression” of the “artificial marker” Tregs; the machine learning model may be trained to predict the positive expression (Tregs+) of the artificial marker where the combined marker expression is CD25+IL7RA− and negative expression (Tregs−) of the artificial marker where the combined marker expression is CD25−ILR7A−, CD25−LR7a+, and CD25+LR7A+. Examples of artificial markers are listed in Table 11. Negative marker values correspond to all possible combinations of relevant markers from the panel except those specified in the “Marker Composition for Positive Value” column.

As another example, as shown in Table 2A, a single multi-class classifier may be used to identify cell types for cells labeled with markers in panel 2. In the example of Table 2A, the multi-class classifier is trained to predict the cell type for the cell. It should be appreciated that, instead of using a multi-class classifier, multiple binary classifiers may be used to identify the cell type, each of which trained to predict the probability that the cell is of the types listed in Table 2A.

TABLE 1A
Example panel of markers and machine learning models used to
predict cell types for cells labeled with the panel of markers.
Panel 1

| | |
|---|---|
| Markers | ICOS, CD8, Tim-3, gdTCR, CD4, CD19, CD13, CD3, CD62L, CD27, |
| | Lag-3, TIGIT, PD-1, CD39, CD45RA |
| Model Type | Trained To Predict |
| Binary Classifier | Degree of expression of marker: CD8 |
| Binary Classifier | Degree of expression of marker: CD27 |
| Binary Classifier | Degree of expression of marker: CD39 |
| Binary Classifier | Degree of expression of marker: CD45RA |
| Binary Classifier | Degree of expression of marker: CD62L |
| Binary Classifier | Degree of expression of marker: PD-1 |
| Binary Classifier | Degree of expression of marker: TIGIT |
| Binary Classifier | Degree of expression of marker: Tim-3 |

TABLE 1B
Marker expression used to identify cell types for cells labeled with markers of panel 1.
Panel 1

| Markers | Cell Types |
|---|---|
| CD8+, CD27+, CD45RA−, CD62L+, CD39+, PD-1+, | CD8 Central Memory CD39+ PD-1+ TIGIT+ Tim-3− |
| TIGIT+, Tim-3− | |
| CD8+, CD27+, CD45RA−, CD62L+, CD39−, PD-1+, | CD8 Central Memory CD39− PD-1+ TIGIT+ Tim-3− |
| TIGIT+, Tim-3− | |
| CD8+, CD27+, CD45RA−, CD62L+, CD39−, PD-1+, | CD8 Central Memory CD39− PD-1+ TIGIT− Tim-3− |
| TIGIT−, Tim-3− | |
| CD8+, CD27+, CD45RA−, CD62L+, CD39+, PD-1−, | CD8 Central Memory CD39+ PD-1− TIGIT+ Tim-3− |
| TIGIT+, Tim-3− | |
| CD8+, CD27+, CD45RA−, CD62L+, CD39+, PD-1−, | CD8 Central Memory CD39+ PD-1− TIGIT− Tim-3− |
| TIGIT−, Tim-3− | |
| CD8+, CD27+, CD45RA−, CD62L+, CD39−, PD-1−, | CD8 Central Memory CD39− PD-1− TIGIT+ Tim-3− |
| TIGIT+, Tim-3− | |
| CD8+, CD27+, CD45RA−, CD62L+, CD39−, PD-1−, | CD8 Central Memory CD39− PD-1− TIGIT− Tim-3+ |
| TIGIT−, Tim-3+ | |
| CD8+, CD27+, CD45RA−, CD62L+, CD39−, PD-1−, | CD8 Central Memory CD39− PD-1− TIGIT− Tim-3− |
| TIGIT−, Tim-3− | |
| CD8+, CD27−, CD45RA−, CD62L−, CD39+, PD-1+, | CD8 Effector Memory CD39+ PD-1+ TIGIT+ Tim-3− |
| TIGIT+, Tim-3− | |
| CD8+, CD27−, CD45RA−, CD62L−, CD39+, PD-1+, | CD8 Effector Memory CD39+ PD-1+ TIGIT− Tim-3− |
| TIGIT−, Tim-3− | |
| CD8+, CD27−, CD45RA−, CD62L−, CD39−, PD-1+, | CD8 Effector Memory CD39− PD-1+ TIGIT+ Tim-3− |
| TIGIT+, Tim-3− | |
| CD8+, CD27−, CD45RA−, CD62L−, CD39−, PD-1+, | CD8 Effector Memory CD39− PD-1+ TIGIT− Tim-3− |
| TIGIT−, Tim-3− | |
| CD8+, CD27−, CD45RA−, CD62L−, CD39+, PD-1−, | CD8 Effector Memory CD39+ PD-1− TIGIT+ Tim-3− |
| TIGIT+, Tim-3− | |
| CD8+, CD27−, CD45RA−, CD62L−, CD39+, PD-1−, | CD8 Effector Memory CD39+ PD-1− TIGIT− Tim-3− |
| TIGIT−, Tim-3− | |
| CD8+, CD27−, CD45RA−, CD62L−, CD39−, PD-1−, | CD8 Effector Memory CD39− PD-1− TIGIT+ Tim-3− |
| TIGIT+, Tim-3− | |
| CD8+, CD27−, CD45RA−, CD62L−, CD39−, PD-1−, | CD8 Effector Memory CD39− PD-1− TIGIT− Tim-3− |
| TIGIT−, Tim-3− | |
| CD8+, CD27+, CD45RA−, CD62L−, CD39+, PD-1+, | CD8 Transitional Memory CD39+ PD-1+ TIGIT+ |
| TIGIT+, Tim-3− | Tim-3− |
| CD8+, CD27+, CD45RA−, CD62L−, CD39+, PD-1+, | CD8 Transitional Memory CD39+ PD-1+ TIGIT− |
| TIGIT−, Tim-3− | Tim-3− |
| CD8+, CD27+, CD45RA−, CD62L−, CD39−, PD-1+, | CD8 Transitional Memory CD39− PD-1+ TIGIT+ |
| TIGIT+, Tim-3− | Tim-3− |
| CD8+, CD27+, CD45RA−, CD62L−, CD39−, PD-1+, | CD8 Transitional Memory CD39− PD-1+ TIGIT− |
| TIGIT−, Tim-3− | Tim-3− |
| CD8+, CD27+, CD45RA−, CD62L−, CD39+, PD-1−, | CD8 Transitional Memory CD39+ PD-1− TIGIT+ |
| TIGIT+, Tim-3− | Tim-3− |
| CD8+, CD27+, CD45RA−, CD62L−, CD39+, PD-1−, | CD8 Transitional Memory CD39+ PD-1− TIGIT− |
| TIGIT−, Tim-3− | Tim-3− |
| CD8+, CD27+, CD45RA−, CD62L−, CD39−, PD-1−, | CD8 Transitional Memory CD39− PD-1− TIGIT+ |
| TIGIT+, Tim-3− | Tim-3− |
| CD8+, CD27+, CD45RA−, CD62L−, CD39−, PD-1−, | CD8 Transitional Memory CD39− PD-1− TIGIT− |
| TIGIT−, Tim-3− | Tim-3− |
| CD8+, CD27−, CD45RA+, CD62L−, CD39+, PD-1+, | CD8 TEMRA CD39+ PD-1+ TIGIT+ Tim-3− |
| TIGIT+, Tim-3− | |
| CD8+, CD27−, CD45RA+, CD62L−, CD39+, PD-1+, | CD8 TEMRA CD39+ PD-1+ TIGIT− Tim-3− |
| TIGIT−, Tim-3− | |
| CD8+, CD27−, CD45RA+, CD62L−, CD39−, PD-1+, | CD8 TEMRA CD39− PD-1+ TIGIT+ Tim-3− |
| TIGIT+, Tim-3− | |
| CD8+, CD27−, CD45RA+, CD62L−, CD39−, PD-1+, | CD8 TEMRA CD39− PD-1+ TIGIT− Tim-3− |
| TIGIT−, Tim-3− | |
| CD8+, CD27−, CD45RA+, CD62L−, CD39+, PD-1−, | CD8 TEMRA CD39+ PD-1− TIGIT+ Tim-3− |
| TIGIT+, Tim-3− | |
| CD8+, CD27−, CD45RA+, CD62L−, CD39+, PD-1−, | CD8 TEMRA CD39+ PD-1− TIGIT− Tim-3− |
| TIGIT−, Tim-3− | |
| CD8+, CD27−, CD45RA+, CD62L−, CD39−, PD-1−, | CD8 TEMRA CD39− PD-1− TIGIT+ Tim-3− |
| TIGIT+, Tim-3− | |
| CD8+, CD27−, CD45RA+, CD62L−, CD39−, PD-1−, | CD8 TEMRA CD39− PD-1− TIGIT− Tim-3− |
| TIGIT−, Tim-3− | |
| CD8+, CD27+, CD45RA+, CD62L−, CD39−, PD-1+, | CD8 Memory CD27+ CD45RA+ CD62L− CD39− |
| TIGIT+, Tim-3− | PD-1+ TIGIT+ Tim-3− |
| CD8+, CD27+, CD45RA+, CD62L−, CD39−, PD-1+, | CD8 Memory CD27+ CD45RA+ CD62L− CD39− |
| TIGIT−, Tim-3− | PD-1+ TIGIT− Tim-3− |
| CD8+, CD27+, CD45RA+, CD62L−, CD39+, PD-1−, | CD8 Memory CD27+ CD45RA+ CD62L− CD39+ |
| TIGIT−, Tim-3− | PD-1− TIGIT− Tim-3− |
| CD8+, CD27+, CD45RA+, CD62L−, CD39−, PD-1−, | CD8 Memory CD27+ CD45RA+ CD62L− CD39− |
| TIGIT+, Tim-3− | PD-1− TIGIT+ Tim-3− |
| CD8+, CD27+, CD45RA+, CD62L−, CD39−, PD-1−, | CD8 Memory CD27+ CD45RA+ CD62L− CD39− |
| TIGIT−, Tim-3− | PD-1− TIGIT− Tim-3− |
| CD8+, CD27−, CD45RA+, CD62L+, CD39−, PD-1+, | CD8 Memory CD27− CD45RA+ CD62L+ CD39− |
| TIGIT+, Tim-3− | PD-1+ TIGIT+ Tim-3− |
| CD8+, CD27−, CD45RA+, CD62L+, CD39−, PD-1+, | CD8 Memory CD27− CD45RA+ CD62L+ CD39− |
| TIGIT−, Tim-3− | PD-1+ TIGIT− Tim-3− |
| CD8+, CD27−, CD45RA+, CD62L+, CD39+, PD-1−, | CD8 Memory CD27− CD45RA+ CD62L+ CD39+ |
| TIGIT+, Tim-3− | PD-1− TIGIT+ Tim-3− |
| CD8+, CD27−, CD45RA+, CD62L+, CD39−, PD-1−, | CD8 Memory CD27− CD45RA+ CD62L+ CD39− |
| TIGIT+, Tim-3− | PD-1− TIGIT+ Tim-3− |
| CD8+, CD27−, CD45RA+, CD62L+, CD39−, PD-1−, | CD8 Memory CD27− CD45RA+ CD62L+ CD39− |
| TIGIT−, Tim-3− | PD-1− TIGIT− Tim-3− |
| CD8+, CD27−, CD45RA−, CD62L+, CD39−, PD-1+, | CD8 Memory CD27− CD45RA− CD62L+ CD39− |
| TIGIT+, Tim-3− | PD-1+ TIGIT+ Tim-3− |
| CD8+, CD27−, CD45RA−, CD62L+, CD39−, PD-1+, | CD8 Memory CD27− CD45RA− CD62L+ CD39− |
| TIGIT−, Tim-3− | PD-1+ TIGIT− Tim-3− |
| CD8+, CD27−, CD45RA−, CD62L+, CD39−, PD-1−, | CD8 Memory CD27− CD45RA− CD62L+ CD39− |
| TIGIT+, Tim-3− | PD-1− TIGIT+ Tim-3− |
| CD8+, CD27−, CD45RA−, CD62L+, CD39−, PD-1−, | CD8 Memory CD27− CD45RA− CD62L+ CD39− |
| TIGIT−, Tim-3− | PD-1− TIGIT− Tim-3− |
| CD8+, CD27+, CD45RA+, CD62L+, CD39+, PD-1−, | CD8 Naive CD39+ PD-1− TIGIT+ Tim-3− |
| TIGIT+, Tim-3− | |
| CD8+, CD27+, CD45RA+, CD62L+, CD39+, PD-1−, | CD8 Naive CD39 PD-1− TIGIT− Tim-3− |
| TIGIT−, Tim-3− | |
| CD8+, CD27+, CD45RA+, CD62L+, CD39−, PD-1−, | CD8 Naive CD39− PD-1− TIGIT+ Tim-3− |
| TIGIT+, Tim-3− | |
| CD8+, CD27+, CD45RA+, CD62L+, CD39−, PD-1−, | CD8 Naive CD39− PD-1− TIGIT− Tim-3+ |
| TIGIT−, Tim-3+ | |
| CD8+, CD27+, CD45RA+, CD62L+, CD39−, PD-1−, | CD8 Naive CD39− PD-1− TIGIT− Tim-3− |
| TIGIT−, Tim-3− | |

TABLE 2A
Example panel of markers and machine learning models used to
predict cell types for cells labeled with the panel of markers.
Panel 2

| | |
|---|---|
| Markers | CD4, CCR6, CXCR3, gdTCR, CD8, CD19, CD13, CD3, CD62L, CD27, |
| | CD45RA, IL-7RA, CCR4, CXCR5, CD25 |
| Model Type | Trained to Predict |
| Multi-class Classifier | Cell types from among: |
| | CD4 Central Memory CCR4− CCR6+ CXCR3− CXCR5+ |
| | CD4 Central Memory CCR4− CCR6+ CXCR3− CXCR5− |
| | CD4 Central Memory CCR4− CCR6− CXCR3− CXCR5+ |
| | CD4 Central Memory CCR4− CCR6− CXCR3− CXCR5− |
| | CD4 Central Memory CCR4+ CCR6+ CXCR3− CXCR5+ |
| | CD4 Central Memory CCR4+ CCR6+ CXCR3− CXCR5− |
| | CD4 Central Memory CCR4+ CCR6− CXCR3− CXCR5+ |
| | CD4 Central Memory CCR4+ CCR6− CXCR3− CXCR5− |
| | CD4 Central Memory CCR4+ CCR6+ CXCR3+ CXCR5− |
| | CD4 Central Memory CCR4+ CCR6+ CXCR3+ CXCR5+ |
| | CD4 Central Memory CCR4+ CCR6− CXCR3+ CXCR5− |
| | CD4 Central Memory CCR4+ CCR6− CXCR3+ CXCR5+ |
| | CD4 Central Memory CCR4− CCR6+ CXCR3+ CXCR5− |
| | CD4 Central Memory CCR4− CCR6+ CXCR3+ CXCR5+ |
| | CD4 Central Memory CCR4− CCR6− CXCR3+ CXCR5+ |
| | CD4 Central Memory CCR4− CCR6− CXCR3+ CXCR5− |
| | CD4 Transitional Memory CCR4+ CCR6− CXCR3+ CXCR5− |
| | CD4 Transitional Memory CCR4+ CCR6− CXCR3+ CXCR5+ |
| | CD4 Transitional Memory CCR4+ CCR6+ CXCR3+ CXCR5− |
| | CD4 Transitional Memory CCR4+ CCR6+ CXCR3+ CXCR5+ |
| | CD4 Transitional Memory CCR4− CCR6+ CXCR3+ CXCR5+ |
| | CD4 Transitional Memory CCR4− CCR6+ CXCR3+ CXCR5− |
| | CD4 Transitional Memory CCR4− CCR6− CXCR3+ CXCR5− |
| | CD4 Transitional Memory CCR4− CCR6− CXCR3+ CXCR5+ |
| | CD4 Transitional Memory CCR4− CCR6+ CXCR3− CXCR5+ |
| | CD4 Transitional Memory CCR4− CCR6+ CXCR3− CXCR5− |
| | CD4 Transitional Memory CCR4− CCR6− CXCR3− CXCR5+ |
| | CD4 Transitional Memory CCR4− CCR6− CXCR3− CXCR5− |
| | CD4 Transitional Memory CCR4+ CCR6+ CXCR3− CXCR5− |
| | CD4 Transitional Memory CCR4+ CCR6+ CXCR3− CXCR5+ |
| | CD4 Transitional Memory CCR4+ CCR6− CXCR3− CXCR5+ |
| | CD4 Transitional Memory CCR4+ CCR6− CXCR3− CXCR5− |
| | CD4 Effector Memory CCR4− CCR6− CXCR3+ CXCR5− |
| | CD4 Effector Memory CCR4+ CCR6− CXCR3− CXCR5− |
| | CD4 Effector Memory CCR4− CCR6− CXCR3− CXCR5− |
| | CD4 Other Effector Memory |
| | CD4 Effector Memory CCR4+ CCR6+ CXCR3− CXCR5− |
| | CD4 Memory CD27− CD45RA− CD62L+ |
| | CD4 TEMRA |
| | CD4 Naive T Cells |
| | CD4 Memory CD27+ CD45RA+ CD62L− |
| | CD4 Naive Tregs |
| | CD4 Memory Tregs |
| | CD4− T Cells |
| | T Cells & Undefined |

TABLE 2B
Example panel of markers and machine learning models used to
predict cell types for cells labeled with the panel of markers.
Panel 2

| | |
|---|---|
| Markers | CD4, CCR6, CXCR3, gdTCR, CD8, CD19, CD13, CD3, CD62L, CD27, |
| | CD45RA, IL-7RA, CCR4, CXCR5, CD25 |
| Model Type | Trained To Predict |
| Binary Classifier | Degree of expression of marker: CCR4 |
| Binary Classifier | Degree of expression of marker: CCR6 |
| Binary Classifier | Degree of expression of marker: CD27 |
| Binary Classifier | Degree of expression of marker: CD4 |
| Binary Classifier | Degree of expression of marker: CD45RA |
| Binary Classifier | Degree of expression of marker: CD62L |
| Binary Classifier | Degree of expression of marker: CXCR3 |
| Binary Classifier | Degree of expression of marker: CXCR5 |
| Binary Classifier | Degree of expression of marker: |
| | Tregs (IL-7RA, CD25) |

TABLE 2C
Marker expression used to identify cell types for cells labeled with markers of panel 2.
Panel 2

| Markers | Cell Types |
|---|---|
| CD4+, Tregs+, CD45RA+, CD27+, CD62L+ | CD4 Naïve Tregs |
| CD4+, Tregs+, CD62L+ | CD4 Memory Tregs CD62L+ |
| CD4+, Tregs+, CD62L− | CD4 Memory Tregs CD62L− |
| CD4+, Tregs−, CD45RA+, CD27−, CD62L+ | CD4 TEMRA CD62L+ |
| CD4+, Tregs−, CD45RA+, CD27−, CD62L− | CD4 TEMRA CD62L− |
| CD4+, Tregs−, CD45RA+, CD27+, CD62L+ | CD4_Naive_T cells |
| CD4+, Tregs−, CD45RA+, CD27+, CD62L− | CD4 Memory CD27+ CD45RA+ CD62L− |
| CD4+, Tregs−, CD45RA−, CD27+, CD62L+, | CD4 Central Memory CCR4+ CCR6 CXCR3+ |
| CXCR3+, CCR4+, CCR6−, CXCR5+ | CXCR5+ |
| CD4+, Tregs−, CD45RA−, CD27+, CD62L+, | CD4 Central Memory CCR4+ CCR6− CXCR3+ |
| CXCR3+, CCR4+, CCR6−, CXCR5− | CXCR5− |
| CD4+, Tregs−, CD45RA−, CD27+, CD62L+, | CD4 Central Memory CCR4+ CCR6+ CXCR3+ |
| CXCR3+, CCR4+, CCR6+, CXCR5+ | CXCR5+ |
| CD4+, Tregs−, CD45RA−, CD27+, CD62L+, | CD4 Central Memory CCR4+ CCR6+ CXCR3+ |
| CXCR3+, CCR4+, CCR6+, CXCR5− | CXCR5− |
| CD4+, Tregs−, CD45RA−, CD27+, CD62L+, | CD4 Central Memory CCR4− CCR6− CXCR3+ |
| CXCR3+, CCR4−, CCR6−, CXCR5− | CXCR5− |
| CD4+, Tregs−, CD45RA−, CD27+, CD62L+, | CD4 Central Memory CCR4− CCR6− CXCR3+ |
| CXCR3+, CCR4−, CCR6−, CXCR5+ | CXCR5+ |
| CD4+, Tregs−, CD45RA−, CD27+, CD62L+, | CD4 Central Memory CCR4− CCR6+ CXCR3+ |
| CXCR3+, CCR4−, CCR6+, CXCR5− | CXCR5− |
| CD4+, Tregs−, CD45RA−, CD27+, CD62L+, | CD4 Central Memory CCR4− CCR6+ CXCR3+ |
| CXCR3+, CCR4−, CCR6+, CXCR5+ | CXCR5+ |
| CD4+, Tregs−, CD45RA−, CD27+, CD62L+, | CD4 Central Memory CCR4+ CCR6+ CXCR3− |
| CXCR3−, CCR4+, CCR6+, CXCR5+ | CXCR5+ |
| CD4+, Tregs−, CD45RA−, CD27+, CD62L+, | CD4 Central Memory CCR4+ CCR6+ CXCR3− |
| CXCR3−, CCR4+, CCR6+, CXCR5− | CXCR5− |
| CD4+, Tregs−, CD45RA−, CD27+, CD62L+, | CD4 Central Memory CCR4+ CCR6− CXCR3− |
| CXCR3−, CCR4+, CCR6−, CXCR5+ | CXCR5+ |
| CD4+, Tregs−, CD45RA−, CD27+, CD62L+, | CD4 Central Memory CCR4+ CCR6− CXCR3− |
| CXCR3−, CCR4+, CCR6−, CXCR5− | CXCR5− |
| CD4+, Tregs−, CD45RA−, CD27+, CD62L+, | CD4 Central Memory CCR4− CCR6+ CXCR3− |
| CXCR3−, CCR4−, CCR6+, CXCR5− | CXCR5− |
| CD4+, Tregs−, CD45RA−, CD27+, CD62L+, | CD4 Central Memory CCR4− CCR6+ CXCR3− |
| CXCR3−, CCR4−, CCR6+, CXCR5+ | CXCR5+ |
| CD4+, Tregs−, CD45RA−, CD27+, CD62L+, | CD4 Central Memory CCR4− CCR6− CXCR3− |
| CXCR3−, CCR4−, CCR6−, CXCR5− | CXCR5− |
| CD4+, Tregs−, CD45RA−, CD27+, CD62L+, | CD4 Central Memory CCR4− CCR6− CXCR3− |
| CXCR3−, CCR4−, CCR6−, CXCR5+ | CXCR5+ |
| CD4+, Tregs−, CD45RA−, CD27+, CD62L−, | CD4 Transitional Memory CCR4+ CCR6− |
| CXCR3+, CCR4+, CCR6−, CXCR5+ | CXCR3+ CXCR5+ |
| CD4+, Tregs−, CD45RA−, CD27+, CD62L−, | CD4 Transitional Memory CCR4+ CCR6− |
| CXCR3+, CCR4+, CCR6−, CXCR5− | CXCR3+ CXCR5− |
| CD4+, Tregs−, CD45RA−, CD27+, CD62L−, | CD4 Transitional Memory CCR4+ CCR6+ |
| CXCR3+, CCR4+, CCR6+, CXCR5+ | CXCR3+ CXCR5+ |
| CD4+, Tregs−, CD45RA−, CD27+, CD62L−, | CD4 Transitional Memory CCR4+ CCR6+ |
| CXCR3+, CCR4+, CCR6+, CXCR5− | CXCR3+ CXCR5− |
| CD4+, Tregs−, CD45RA−, CD27+, CD62L−, | CD4 Transitional Memory CCR4− CCR6− CXCR3+ |
| CXCR3+, CCR4−, CCR6−, CXCR5− | CXCR5− |
| CD4+, Tregs−, CD45RA−, CD27+, CD62L−, | CD4 Transitional Memory CCR4− CCR6− CXCR3+ |
| CXCR3+, CCR4−, CCR6−, CXCR5+ | CXCR5+ |
| CD4+, Tregs−, CD45RA−, CD27+, CD62L−, | CD4 Transitional Memory CCR4− CCR6+ |
| CXCR3+, CCR4−, CCR6+, CXCR5− | CXCR3+ CXCR5− |
| CD4+, Tregs−, CD45RA−, CD27+, CD62L−, | CD4 Transitional Memory CCR4− CCR6+ |
| CXCR3+, CCR4−, CCR6+, CXCR5+ | CXCR3+ CXCR5+ |
| CD4+, Tregs−, CD45RA−, CD27+, CD62L−, | CD4 Transitional Memory CCR4+ CCR6+ |
| CXCR3−, CCR4+, CCR6+, CXCR5+ | CXCR3− CXCR5+ |
| CD4+, Tregs−, CD45RA−, CD27+, CD62L−, | CD4 Transitional Memory CCR4+ CCR6+ |
| CXCR3−, CCR4+, CCR6+, CXCR5− | CXCR3− CXCR5− |
| CD4+, Tregs−, CD45RA−, CD27+, CD62L−, | CD4 Transitional Memory CCR4+ CCR6− CXCR3− |
| CXCR3−, CCR4+, CCR6−, CXCR5+ | CXCR5+ |
| CD4+, Tregs−, CD45RA−, CD27+, CD62L−, | CD4 Transitional Memory CCR4+ CCR6− CXCR3− |
| CXCR3−, CCR4+, CCR6−, CXCR5− | CXCR5− |
| CD4+, Tregs−, CD45RA−, CD27+, CD62L−, | CD4 Transitional Memory CCR4− CCR6+ CXCR3− |
| CXCR3−, CCR4−, CCR6+, CXCR5− | CXCR5− |
| CD4+, Tregs−, CD45RA−, CD27+, CD62L−, | CD4 Transitional Memory CCR4− CCR6+ CXCR3− |
| CXCR3−, CCR4−, CCR6+, CXCR5+ | CXCR5+ |
| CD4+, Tregs−, CD45RA−, CD27+, CD62L−, | CD4 Transitional Memory CCR4− CCR6− CXCR3− |
| CXCR3−, CCR4−, CCR6−, CXCR5− | CXCR5− |
| CD4+, Tregs−, CD45RA−, CD27+, CD62L−, | CD4 Transitional Memory CCR4− CCR6− CXCR3− |
| CXCR3−, CCR4−, CCR6−, CXCR5+ | CXCR5+ |
| CD4+, Tregs−, CD45RA−, CD27−, CD62L+ | CD4 Memory CD27− CD45RA− CD62L+ |
| CD4+, Tregs−, CD45RA−, CD27−, CD62L−, | CD4 Effector Memory CCR4+ CCR6+ CXCR3− |
| CCR4+, CCR6+, CXCR3−, CXCR5− | CXCR5− |
| CD4+, Tregs−, CD45RA−, CD27−, CD62L−, | CD4 Effector Memory CCR4+ CCR6− CXCR3− |
| CCR4+, CCR6−, CXCR3−, CXCR5− | CXCR5− |
| CD4+, Tregs−, CD45RA−, CD27−, CD62L−, | CD4 Effector Memory CCR4− CCR6− CXCR3+ |
| CCR4−, CCR6−, CXCR3+, CXCR5− | CXCR5− |
| CD4+, Tregs−, CD45RA−, CD27−, CD62L−, | CD4 Effector Memory CCR4− CCR6− CXCR3− |
| CCR4−, CCR6−, CXCR3−, CXCR5− | CXCR5− |

TABLE 3
Example panel of markers and machine learning models used to
predict cell types for cells labeled with the panel of markers.
Panel 3

| | |
|---|---|
| Markers | CD14, CD13, CCR3, CD123, HLA-DR, CD3, CD45, CD66b, CD56, |
| | CD11b, CD19 |
| Model Type | Trained to Predict |
| Multi-class Classifier | Cell types from among: |
| | Neutrophils |
| | Other Granulocytes |
| | B cells |
| | NK cells |
| | HLA-DR- T-cells |
| | NKT cells |
| | HLA-DR+ T-cells |
| | Monocytes |
| | pDC |
| | cDC |
| | Other Leukocytes |
| | Basophils |
| | Eosinophils |

TABLE 4
Example panel of markers and machine learning models used to predict
cell types for cells labeled with the panel of markers.
Panel 4

| | |
|---|---|
| Markers | FceR1, CD13, CD1c, CD15, CD3, CD19, |
| | CCR3, CD7, CD123, HLA-DR, CD16, |
| | CD45, CLEC9A, CD141, CD11c, CD14 |
| Model Type | Trained to Predict |
| Multi-class | Cell types from among: |
| Classifier | Other Leukocytes |
| | cDC1 |
| | cDC2 |
| | pDC |
| | Granulocytes |

TABLE 5
Example panel of markers and machine learning models used to predict
cell types for cells labeled with the panel of markers.
Panel 5

| | |
|---|---|
| Markers | CD19, IgD, CD138, CD3, CD7, CD13, IgG, |
| | CD39, CD24, CD10, IgA, IgM, CD27, CD38 |
| Model Type | Trained to Predict |
| Multi-class | Cell types from among: |
| Classifier | Naïve B cells |
| | Non-switched Memory IgM B cells |
| | CD27− Memory B cells |
| | Switched Memory IgG+ |
| | Switched Memory IgA+ |
| | Switched Memory IgA− IgG− |
| | Plasmablasts IgA+ |
| | Plasmablasts IgG+ |
| | Plasmablasts IgA− IgG− |
| | Plasma cells IgA+ |
| | Plasma cells IgG+ |
| | Plasma cells IgA− IgG− |

TABLE 6
Example panel of markers and machine learning models used to predict
cell types for cells labeled with the panel of markers.
Panel 6

| | |
|---|---|
| Markers | CD14, CD9, CD16, CD3, CCR3, CD19, CD7, FceR1, |
| | HLA-DR, CD33, CD45, CD84, CD15, CD169, CD206 |
| Model Type | Trained to Predict |
| Multi-class | Cell types from among: |
| Classifier | Granulocytes |
| | Other Leukocytes |
| | Classical Monocytes FceRI+ |
| | Classical Monocytes FceRI− |
| | Non-classical Monocytes |
| | HLA-DR-low Monocytes |

TABLE 7A
Example panel of markers and machine learning models used to predict
cell types for cells labeled with the panel of markers.
Panel 7

| | |
|---|---|
| Markers | CD27, CD8, CXCR3, TCRgd, CD4, CD19, CD13, CD3, CD62L, CD95, |
| | CD57, CX3CR1, PD-1, CXCR5, CD45RA |
| Model Type | Trained to Predict |
| Binary Classifier | Degree of expression of marker: CD8 |
| Binary Classifier | Degree of expression of marker: CD27 |
| Binary Classifier | Degree of expression of marker: CD45A |
| Binary Classifier | Degree of expression of marker: CD57 |
| Binary Classifier | Degree of expression of marker: CD62L |
| Binary Classifier | Degree of expression of marker: CX3CR1 |
| Binary Classifier | Degree of expression of marker: CXCR3 |
| Binary Classifier | Degree of expression of marker: CXCR5 |
| Binary Classifier | Degree of expression of marker: PD-1 |

TABLE 7B
Marker expression used to identify cell types for cells labeled with markers of panel 7.
Panel 7

| Marker Expression | Cell Types |
|---|---|
| CD8+, CD27+, CD45RA−, CD62L+, CD57+, | CD8 Central Memory CD57+ CX3CR1− CXCR3+ |
| CX3CR1−, CXCR3+, CXCR5+, PD-1+ | CXCR5+ PD-1+ |
| CD8+, CD27+, CD45RA−, CD62L+, CD57+, | CD8 Central Memory CD57+ CX3CR1+ CXCR3+ |
| CX3CR1+, CXCR3+, CXCR5−, PD-1+ | CXCR5− PD-1+ |
| CD8+, CD27+, CD45RA−, CD62L+, CD57+, | CD8 Central Memory CD57+ CX3CR1+ CXCR3+ |
| CX3CR1+, CXCR3+, CXCR5−, PD-1− | CXCR5− PD-1− |
| CD8+, CD27+, CD45RA−, CD62L+, CD57+, | CD8 Central Memory CD57+ CX3CR1+ CXCR3− |
| CX3CR1+, CXCR3−, CXCR5−, PD-1+ | CXCR5− PD-1+ |
| CD8+, CD27+, CD45RA−, CD62L+, CD57+, | CD8 Central Memory CD57+ CX3CR1+ CXCR3− |
| CX3CR1+, CXCR3−, CXCR5−, PD-1− | CXCR5− PD-1− |
| CD8+, CD27+, CD45RA−, CD62L+, CD57−, | CD8 Central Memory CD57− CX3CR1+ CXCR3+ |
| CX3CR1+, CXCR3+, CXCR5−, PD-1+ | CXCR5− PD-1+ |
| CD8+, CD27+, CD45RA−, CD62L+, CD57−, | CD8 Central Memory CD57− CX3CR1+ CXCR3+ |
| CX3CR1+, CXCR3+, CXCR5−, PD-1− | CXCR5− PD-1− |
| CD8+, CD27+, CD45RA−, CD62L+, CD57−, | CD8 Central Memory CD57− CX3CR1+ CXCR3− |
| CX3CR1+, CXCR3−, CXCR5−, PD-1+ | CXCR5− PD-1+ |
| CD8+, CD27+, CD45RA−, CD62L+, CD57−, | CD8 Central Memory CD57− CX3CR1+ CXCR3− |
| CX3CR1+, CXCR3−, CXCR5−, PD-1− | CXCR5− PD-1− |
| CD8+, CD27+, CD45RA−, CD62L+, CD57+, | CD8 Central Memory CD57+ CX3CR1− CXCR3+ |
| CX3CR1−, CXCR3+, CXCR5−, PD-1+ | CXCR5− PD-1+ |
| CD8+, CD27+, CD45RA−, CD62L+, CD57+, | CD8 Central Memory CD57+ CX3CR1− CXCR3+ |
| CX3CR1−, CXCR3+, CXCR5−, PD-1− | CXCR5− PD-1− |
| CD8+, CD27+, CD45RA−, CD62L+, CD57−, | CD8 Central Memory CD57− CX3CR1−CXCR3− |
| CX3CR1−, CXCR3−, CXCR5−, PD-1+ | CXCR5− PD-1+ |
| CD8+, CD27+, CD45RA−, CD62L+, CD57−, | CD8 Central Memory CD57− CX3CR1− CXCR3− |
| CX3CR1−, CXCR3−, CXCR5−, PD-1− | CXCR5− PD-1− |
| CD8+, CD27+, CD45RA−, CD62L+, CD57−, | CD8 Central Memory CD57− CX3CR1− CXCR3+ |
| CX3CR1−, CXCR3+, CXCR5+, PD-1+ | CXCR5+ PD-1+ |
| CD8+, CD27+, CD45RA−, CD62L+, CD57−, | CD8 Central Memory CD57− CX3CR1− CXCR3+ |
| CX3CR1−, CXCR3+, CXCR5+, PD-1− | CXCR5+ PD-1− |
| CD8+, CD27+, CD45RA−, CD62L+, CD57−, | CD8 Central Memory CD57− CX3CR1− CXCR3+ |
| CX3CR1−, CXCR3+, CXCR5−, PD-1+ | CXCR5− PD-1+ |
| CD8+, CD27+, CD45RA−, CD62L+, CD57−, | CD8 Central Memory CD57− CX3CR1− CXCR3+ |
| CX3CR1−, CXCR3+, CXCR5−, PD-1− | CXCR5− PD-1− |
| CD8+, CD27−, CD45RA−, CD62L−, CD57−, | CD8 Effector Memory CD57− CX3CR1+ CXCR3+ |
| CX3CR1+, CXCR3+, CXCR5−, PD-1+ | CXCR5− PD-1+ |
| CD8+, CD27−, CD45RA−, CD62L−, CD57−, | CD8 Effector Memory CD57− CX3CR1+ CXCR3+ |
| CX3CR1+, CXCR3+, CXCR5−, PD-1− | CXCR5− PD-1− |
| CD8+, CD27−, CD45RA−, CD62L−, CD57+, | CD8 Effector Memory CD57+ CX3CR1− CXCR3− |
| CX3CR1−, CXCR3−, CXCR5−, PD-1− | CXCR5− PD-1− |
| CD8+, CD27−, CD45RA−, CD62L−, CD57+, | CD8 Effector Memory CD57+ CX3CR1+ CXCR3+ |
| CX3CR1+, CXCR3+, CXCR5−, PD-1+ | CXCR5− PD-1+ |
| CD8+, CD27−, CD45RA−, CD62L−, CD57+, | CD8 Effector Memory CD57+ CX3CR1+ CXCR3+ |
| CX3CR1+, CXCR3+, CXCR5−, PD-1− | CXCR5− PD-1− |
| CD8+, CD27−, CD45RA−, CD62L−, CD57+, | CD8 Effector Memory CD57+ CX3CR1+ CXCR3− |
| CX3CR1+, CXCR3−, CXCR5−, PD-1+ | CXCR5− PD-1+ |
| CD8+, CD27−, CD45RA−, CD62L−, CD57+, | CD8 Effector Memory CD57+ CX3CR1+ CXCR3− |
| CX3CR1+, CXCR3−, CXCR5−, PD-1− | CXCR5− PD-1− |
| CD8+, CD27−, CD45RA−, CD62L−, CD57+, | CD8 Effector Memory CD57+ CX3CR1− CXCR3+ |
| CX3CR1−, CXCR3+, CXCR5−, PD-1+ | CXCR5− PD-1+ |
| CD8+, CD27−, CD45RA−, CD62L−, CD57+, | CD8 Effector Memory CD57+ CX3CR1− CXCR3+ |
| CX3CR1−, CXCR3+, CXCR5−, PD-1− | CXCR5− PD-1− |
| CD8+, CD27−, CD45RA−, CD62L−, CD57−, | CD8 Effector Memory CD57− CX3CR1− CXCR3+ |
| CX3CR1−, CXCR3+, CXCR5−, PD-1+ | CXCR5− PD-1+ |
| CD8+, CD27−, CD45RA−, CD62L−, CD57−, | CD8 Effector Memory CD57− CX3CR1− CXCR3+ |
| CX3CR1−, CXCR3+, CXCR5−, PD-1− | CXCR5− PD-1− |
| CD8+, CD27−, CD45RA−, CD62L−, CD57−, | CD8 Effector Memory CD57− CX3CR1− CXCR3− |
| CX3CR1−, CXCR3−, CXCR5−, PD-1+ | CXCR5− PD-1+ |
| CD8+, CD27−, CD45RA−, CD62L−, CD57−, | CD8 Effector Memory CD57− CX3CR1− CXCR3− |
| CX3CR1−, CXCR3−, CXCR5−, PD-1− | CXCR5− PD-1− |
| CD8+, CD27−, CD45RA+, CD62L−, CD57+, | CD8 TEMRA CD57+ CX3CR1− CXCR3+ CXCR5− |
| CX3CR1−, CXCR3+, CXCR5−, PD-1− | PD-1− |
| CD8+, CD27−, CD45RA+, CD62L−, CD57+, | CD8 TEMRA CD57+ CX3CR1− CXCR3− CXCR5− |
| CX3CR1−, CXCR3−, CXCR5−, PD-1− | PD-1− |
| CD8+, CD27−, CD45RA+, CD62L−, CD57+, | CD8 TEMRA CD57+ CX3CR1+ CXCR3+ CXCR5+ |
| CX3CR1+, CXCR3+, CXCR5+, PD-1− | PD-1− |
| CD8+, CD27−, CD45RA+, CD62L−, CD57+, | CD8 TEMRA CD57+ CX3CR1+ CXCR3− CXCR5− |
| CX3CR1+, CXCR3−, CXCR5−, PD-1+ | PD-1+ |
| CD8+, CD27−, CD45RA+, CD62L−, CD57+, | CD8 TEMRA CD57+ CX3CR1+ CXCR3− CXCR5− |
| CX3CR1+, CXCR3−, CXCR5−, PD-1− | PD-1− |
| CD8+, CD27−, CD45RA+, CD62L−, CD57−, | CD8 TEMRA CD57− CX3CR1+ CXCR3+ CXCR5− |
| CX3CR1+, CXCR3+, CXCR5−, PD-1+ | PD-1+ |
| CD8+, CD27−, CD45RA+, CD62L−, CD57−, | CD8 TEMRA CD57− CX3CR1+ CXCR3+ CXCR5− |
| CX3CR1+, CXCR3+, CXCR5−, PD-1− | PD-1− |
| CD8+, CD27−, CD45RA+, CD62L−, CD57−, | CD8 TEMRA CD57− CX3CR1+ CXCR3− CXCR5− |
| CX3CR1+, CXCR3−, CXCR5−, PD-1+ | PD-1+ |
| CD8+, CD27−, CD45RA+, CD62L−, CD57−, | CD8 TEMRA CD57− CX3CR1+ CXCR3− CXCR5− |
| CX3CR1+, CXCR3−, CXCR5−, PD-1− | PD-1− |
| CD8+, CD27−, CD45RA+, CD62L−, CD57−, | CD8 TEMRA CD57− CX3CR1− CXCR3+ CXCR5− |
| CX3CR1−, CXCR3+, CXCR5−, PD-1+ | PD-1+ |
| CD8+, CD27−, CD45RA+, CD62L−, CD57−, | CD8 TEMRA CD57− CX3CR1− CXCR3+ CXCR5− |
| CX3CR1−, CXCR3+, CXCR5−, PD-1− | PD-1− |
| CD8+, CD27−, CD45RA+, CD62L−, CD57−, | CD8 TEMRA CD57− CX3CR1− CXCR3− CXCR5− |
| CX3CR1−, CXCR3−, CXCR5−, PD-1+ | PD-1+ |
| CD8+, CD27−, CD45RA+, CD62L−, CD57−, | CD8 TEMRA CD57− CX3CR1− CXCR3− CXCR5− |
| CX3CR1−, CXCR3−, CXCR5−, PD-1− | PD-1− |
| CD8+, CD27−, CD45RA+, CD62L−, CD57+, | CD8 TEMRA CD57+ CX3CR1+ CXCR3+ CXCR5− |
| CX3CR1+, CXCR3+, CXCR5−, PD-1+ | PD-1+ |
| CD8+, CD27−, CD45RA+, CD62L−, CD57+, | CD8 TEMRA CD57+ CX3CR1+ CXCR3+ CXCR5− |
| CX3CR1+, CXCR3+, CXCR5−, PD-1− | PD-1− |
| CD8+, CD27+, CD45RA−, CD62L−, CD57+, | CD8 Transitional Memory CD57+ CX3CR1+ |
| CX3CR1+, CXCR3+, CXCR5−, PD-1+ | CXCR3+ CXCR5− PD-1+ |
| CD8+, CD27+, CD45RA−, CD62L−, CD57+, | CD8 Transitional Memory CD57+ CX3CR1+ |
| CX3CR1+, CXCR3+, CXCR5−, PD-1− | CXCR3+ CXCR5− PD-1− |
| CD8+, CD27+, CD45RA−, CD62L−, CD57+, | CD8 Transitional Memory CD57+ CX3CR1+ CXCR3− |
| CX3CR1+, CXCR3−, CXCR5−, PD-1+ | CXCR5− PD-1+ |
| CD8+, CD27+, CD45RA−, CD62L−, CD57+, | CD8 Transitional Memory CD57+ CX3CR1+ CXCR3− |
| CX3CR1+, CXCR3−, CXCR5−, PD-1− | CXCR5− PD-1− |
| CD8+, CD27+, CD45RA−, CD62L−, CD57−, | CD8 Transitional Memory CD57− CX3CR1+ CXCR3+ |
| CX3CR1+, CXCR3+, CXCR5+, PD-1+ | CXCR5+ PD-1+ |
| CD8+, CD27+, CD45RA−, CD62L−, CD57−, | CD8 Transitional Memory CD57− CX3CR1+ CXCR3− |
| CX3CR1+, CXCR3−, CXCR5−, PD-1+ | CXCR5− PD-1+ |
| CD8+, CD27+, CD45RA−, CD62L−, CD57−, | CD8 Transitional Memory CD57− CX3CR1+ CXCR3− |
| CX3CR1+, CXCR3−, CXCR5−, PD-1− | CXCR5− PD-1− |
| CD8+, CD27+, CD45RA−, CD62L−, CD57+, | CD8 Transitional Memory CD57+ CX3CR1− CXCR3+ |
| CX3CR1−, CXCR3+, CXCR5+, PD-1+ | CXCR5+ PD-1+ |
| CD8+, CD27+, CD45RA−, CD62L−, CD57+, | CD8 Transitional Memory CD57+ CX3CR1− CXCR3− |
| CX3CR1−, CXCR3−, CXCR5−, PD-1+ | CXCR5− PD-1+ |
| CD8+, CD27+, CD45RA−, CD62L−, CD57+, | CD8 Transitional Memory CD57+ CX3CR1− CXCR3− |
| CX3CR1−, CXCR3−, CXCR5−, PD-1− | CXCR5− PD-1− |
| CD8+, CD27+, CD45RA−, CD62L−, CD57−, | CD8 Transitional Memory CD57− CX3CR1− CXCR3− |
| CX3CR1−, CXCR3−, CXCR5−, PD-1+ | CXCR5− PD-1+ |
| CD8+, CD27+, CD45RA−, CD62L−, CD57−, | CD8 Transitional Memory CD57− CX3CR1− CXCR3− |
| CX3CR1−, CXCR3−, CXCR5−, PD-1− | CXCR5− PD-1− |
| CD8+, CD27+, CD45RA−, CD62L−, CD57−, | CD8 Transitional Memory CD57− CX3CR1+ CXCR3+ |
| CX3CR1+, CXCR3+, CXCR5−, PD-1+ | CXCR5− PD-1+ |
| CD8+, CD27+, CD45RA−, CD62L−, CD57−, | CD8 Transitional Memory CD57− CX3CR1+ CXCR3+ |
| CX3CR1+, CXCR3+, CXCR5−, PD-1− | CXCR5− PD-1− |
| CD8+, CD27+, CD45RA−, CD62L−, CD57+, | CD8 Transitional Memory CD57+ CX3CR1− CXCR3+ |
| CX3CR1−, CXCR3+, CXCR5−, PD-1+ | CXCR5− PD-1+ |
| CD8+, CD27+, CD45RA−, CD62L−, CD57+, | CD8 Transitional Memory CD57+ CX3CR1− CXCR3+ |
| CX3CR1−, CXCR3+, CXCR5−, PD-1− | CXCR5− PD-1− |
| CD8+, CD27+, CD45RA−, CD62L−, CD57−, | CD8 Transitional Memory CD57− CX3CR1− CXCR3+ |
| CX3CR1−, CXCR3+, CXCR5+, PD-1+ | CXCR5+ PD-1+ |
| CD8+, CD27+, CD45RA−, CD62L−, CD57−, | CD8 Transitional Memory CD57− CX3CR1− CXCR3+ |
| CX3CR1−, CXCR3+, CXCR5+, PD-1− | CXCR5+ PD-1− |
| CD8+, CD27+, CD45RA−, CD62L−, CD57−, | CD8 Transitional Memory CD57− CX3CR1− CXCR3+ |
| CX3CR1−, CXCR3+, CXCR5−, PD-1+ | CXCR5− PD-1+ |
| CD8+, CD27+, CD45RA−, CD62L−, CD57−, | CD8 Transitional Memory CD57− CX3CR1− CXCR3+ |
| CX3CR1−, CXCR3+, CXCR5−, PD-1− | CXCR5−PD-1− |
| CD8+, CD27+, CD45RA+, CD62L+, | CD8 Naïve CD57+ CX3CR1− CXCR3+ CXCR5− |
| CD57+, CX3CR1−, CXCR3+, CXCR5−, | PD-1− |
| PD-1− | |
| CD8+, CD27+, CD45RA+, CD62L+, | CD8 Naïve CD57+ CX3CR1− CXCR3− CXCR5− PD-1− |
| CD57+, CX3CR1−, CXCR3−, CXCR5−, | |
| PD-1− | |
| CD8+, CD27+, CD45RA+, CD62L+, CD57−, | CD8 Naïve CD57− CX3CR1− CXCR3+ CXCR5− PD-1− |
| CX3CR1−, CXCR3+, CXCR5−, PD-1− | |
| CD8+, CD27+, CD45RA+, CD62L+, CD57−, | CD8 Naïve CD57− CX3CR1− CXCR3− CXCR5− PD-1− |
| CX3CR1−, CXCR3−, CXCR5−, PD-1− | |
| CD8+, CD27+, CD45RA+, CD62L−, CD57+, | CD8 Memory CD27+ CD45RA+ CD62L− CD57+ |
| CX3CR1−, CXCR3−, CXCR5−, PD-1+ | CX3CR1− CXCR3− CXCR5− PD-1+ |
| CD8+, CD27+, CD45RA+, CD62L−, CD57+, | CD8 Memory CD27+ CD45RA+ CD62L− CD57+ |
| CX3CR1+, CXCR3+, CXCR5−, PD-1+ | CX3CR1+ CXCR3+ CXCR5− PD-1+ |
| CD8+, CD27+, CD45RA+, CD62L−, CD57+, | CD8 Memory CD27+ CD45RA+ CD62L− CD57+ |
| CX3CR1+, CXCR3+, CXCR5−, PD-1− | CX3CR1+ CXCR3+ CXCR5− PD-1− |
| CD8+, CD27+, CD45RA+, CD62L−, CD57+, | CD8 Memory CD27+ CD45RA+ CD62L−CD57+ |
| CX3CR1+, CXCR3−, CXCR5−, PD-1+ | CX3CR1+ CXCR3− CXCR5− PD-1+ |
| CD8+, CD27+, CD45RA+, CD62L−, CD57+, | CD8 Memory CD27+ CD45RA+ CD62L−CD57+ |
| CX3CR1+, CXCR3−, CXCR5−, PD-1− | CX3CR1+ CXCR3− CXCR5− PD-1− |
| CD8+, CD27+, CD45RA+, CD62L−, CD57−, | CD8 Memory CD27+ CD45RA+ CD62L− CD57− |
| CX3CR1+, CXCR3+, CXCR5−, PD-1+ | CX3CR1+ CXCR3+ CXCR5− PD-1+ |
| CD8+, CD27+, CD45RA+, CD62L−, CD57−, | CD8 Memory CD27+ CD45RA+ CD62L− CD57− |
| CX3CR1+, CXCR3+, CXCR5−, PD-1− | CX3CR1+ CXCR3+ CXCR5− PD-1− |
| CD8+, CD27+, CD45RA+, CD62L−, CD57−, | CD8 Memory CD27+ CD45RA+ CD62L− CD57− |
| CX3CR1+, CXCR3−, CXCR5−, PD-1+ | CX3CR1+ CXCR3− CXCR5− PD-1+ |
| CD8+, CD27+, CD45RA+, CD62L−, CD57−, | CD8 Memory CD27+ CD45RA+ CD62L− CD57− |
| CX3CR1+, CXCR3−, CXCR5−, PD-1− | CX3CR1+ CXCR3− CXCR5− PD-1− |
| CD8+, CD27+, CD45RA+, CD62L−, CD57+, | CD8 Memory CD27+ CD45RA+ CD62L− CD57+ |
| CX3CR1−, CXCR3+, CXCR5−, PD-1+ | CX3CR1− CXCR3+ CXCR5− PD-1+ |
| CD8+, CD27+, CD45RA+, CD62L−, CD57+, | CD8 Memory CD27+ CD45RA+ CD62L− CD57+ |
| CX3CR1−, CXCR3+, CXCR5−, PD-1− | CX3CR1− CXCR3+ CXCR5− PD-1− |
| CD8+, CD27+, CD45RA+, CD62L−, CD57−, | CD8 Memory CD27+ CD45RA+ CD62L− CD57− |
| CX3CR1−, CXCR3+, CXCR5−, PD-1+ | CX3CR1− CXCR3+ CXCR5− PD-1+ |
| CD8+, CD27+, CD45RA+, CD62L−, CD57−, | CD8 Memory CD27+ CD45RA+ CD62L− CD57− |
| CX3CR1−, CXCR3+, CXCR5−, PD-1− | CX3CR1− CXCR3+ CXCR5− PD-1− |
| CD8+, CD27+, CD45RA+, CD62L−, CD57−, | CD8 Memory CD27+ CD45RA+ CD62L− CD57− |
| CX3CR1−, CXCR3−, CXCR5−, PD-1+ | CX3CR1− CXCR3− CXCR5− PD-1+ |
| CD8+, CD27+, CD45RA+, CD62L−, CD57−, | CD8 Memory CD27+ CD45RA+ CD62L− CD57− |
| CX3CR1−, CXCR3−, CXCR5−, PD-1− | CX3CR1− CXCR3− CXCR5− PD-1− |
| CD8+, CD27−, CD45RA+, CD62L+, CD57−, | CD8 Memory CD27− CD45RA+ CD62L+ CD57− |
| CX3CR1+, CXCR3+, CXCR5−, PD-1− | CX3CR1+ CXCR3+ CXCR5− PD-1− |
| CD8+, CD27−, CD45RA+, CD62L+, CD57−, | CD8 Memory CD27− CD45RA+ CD62L+ CD57− |
| CX3CR1+, CXCR3−, CXCR5−, PD-1− | CX3CR1+ CXCR3− CXCR5− PD-1− |
| CD8+, CD27−, CD45RA+, CD62L+, CD57+, | CD8 Memory CD27− CD45RA+ CD62L+ CD57+ |
| CX3CR1+, CXCR3+, CXCR5+, PD-1− | CX3CR1+ CXCR3+ CXCR5+ PD-1− |
| CD8+, CD27−, CD45RA+, CD62L+, CD57+, | CD8 Memory CD27− CD45RA+ CD62L+ CD57+ |
| CX3CR1+, CXCR3−, CXCR5−, PD-1+ | CX3CR1+ CXCR3− CXCR5− PD-1+ |
| CD8+, CD27−, CD45RA+, CD62L+, CD57+, | CD8 Memory CD27− CD45RA+ CD62L+ CD57+ |
| CX3CR1+, CXCR3−, CXCR5−, PD-1− | CX3CR1+ CXCR3− CXCR5− PD-1− |
| CD8+, CD27−, CD45RA+, CD62L+, CD57+, | CD8 Memory CD27− CD45RA+ CD62L+ CD57+ |
| CX3CR1+, CXCR3+, CXCR5−, PD-1+ | CX3CR1+ CXCR3+ CXCR5− PD-1+ |
| CD8+, CD27−, CD45RA+, CD62L+, CD57+, | CD8 Memory CD27− CD45RA+ CD62L+ CD57+ |
| CX3CR1+, CXCR3+, CXCR5−, PD-1− | CX3CR1+ CXCR3+ CXCR5− PD-1− |
| CD8+, CD27−, CD45RA−, CD62L+, CD57+, | CD8 Memory CD27− CD45RA− CD62L+ CD57+ |
| CX3CR1+, CXCR3+, CXCR5−, PD-1+ | CX3CR1+ CXCR3+ CXCR5− PD-1+ |
| CD8+, CD27−, CD45RA−, CD62L+, CD57+, | CD8 Memory CD27− CD45RA−CD62L+ CD57+ |
| CX3CR1+, CXCR3+, CXCR5−, PD-1− | CX3CR1+ CXCR3+ CXCR5− PD-1− |
| CD8+, CD27−, CD45RA−, CD62L+, CD57−, | CD8 Memory CD27− CD45RA− CD62L+ CD57− |
| CX3CR1−, CXCR3+, CXCR5−, PD-1− | CX3CR1− CXCR3+ CXCR5− PD-1− |
| CD8+, CD27−, CD45RA−, CD62L+, CD57−, | CD8 Memory CD27− CD45RA− CD62L+ CD57− |
| CX3CR1−, CXCR3−, CXCR5−, PD-1− | CX3CR1− CXCR3− CXCR5− PD-1− |

TABLE 8A
Example panel of markers and machine learning models used to predict
cell types for cells labeled with the panel of markers.
Panel 8

| | |
|---|---|
| Markers | CTLA-4, CD4, CD25, gdTCR, CD8, CD19, CD13, CD3, ICOS, CD27, |
| | Lag3, IL-7RA, PD-1, CD39, CD45RA |
| Model Type | Trained to Predict |
| Multi-class | Cell types from among: |
| Classifier | CD4 TEMRA ICOS+ PD-1− |
| | CD4 TEMRA ICOS+ PD-1+ |
| | CD4 TEMRA ICOS− PD-1− |
| | CD4 TEMRA ICOS− PD-1+ |
| | CD4 Memory CD39+ ICOS− PD-1− |
| | CD4 Memory CD39+ ICOS− PD-1+ |
| | CD4 Memory CD39+ ICOS+ PD-1+ |
| | CD4 Memory CD39+ ICOS+ PD-1− |
| | CD4 Memory CD39− ICOS− PD-1+ |
| | CD4 Memory CD39− ICOS− PD-1− |
| | CD4 Memory CD39− ICOS+ PD-1− |
| | CD4 Memory CD39− ICOS+ PD-1+ |
| | CD4 Naive CD39+ ICOS+ PD-1+ |
| | CD4 Naive CD39+ ICOS+ PD-1− |
| | CD4 Naive CD39+ ICOS− PD-1+ |
| | CD4 Naive CD39+ ICOS− PD-1− |
| | CD4 Naive CD39− ICOS+ PD-1− |
| | CD4 Naive CD39− ICOS+ PD-1+ |
| | CD4 Naive CD39− ICOS− PD-1+ |
| | CD4 Naive CD39− ICOS− PD-1− |
| | CD4 Naive Tregs CD39− ICOS+ PD-1+ |
| | CD4 Naive Tregs CD39− ICOS+ PD-1− |
| | CD4 Naive Tregs CD39− ICOS− PD-1+ |
| | CD4 Naive Tregs CD39− ICOS− PD-1− |
| | CD4 Naive Tregs CD39+ ICOS+ PD-1− |
| | CD4 Naive Tregs CD39+ ICOS+ PD-1+ |
| | CD4 Naive Tregs CD39+ ICOS− PD-1+ |
| | CD4 Naive Tregs CD39+ ICOS− PD-1− |
| | CD4 Memory Tregs CD39+ ICOS+ PD-1+ |
| | CD4 Memory Tregs CD39+ ICOS+ PD-1− |
| | CD4 Memory Tregs CD39+ ICOS− PD-1+ |
| | CD4 Memory Tregs CD39+ ICOS− PD-1− |
| | CD4 Memory Tregs CD39− ICOS+ PD-1− |
| | CD4 Memory Tregs CD39− ICOS+ PD-1+ |
| | CD4 Memory Tregs CD39− ICOS− PD-1+ |
| | CD4 Memory Tregs CD39− ICOS− PD-1− |
| | CD4− T Cells |
| | T Cells & Undefined |

TABLE 8B
Example panel of markers and machine learning models used to predict
cell types for cells labeled with the panel of markers.
Panel 8

| | |
|---|---|
| Markers | CTLA-4, CD4, CD25, gdTCR, CD8, CD19, CD13, CD3, ICOS, CD27, |
| | Lag3, IL-7RA, PD-1, CD39, CD45RA |
| Artificial Markers | Tregs (IL-7RA, CD25) |
| Model Type | Trained To Predict |
| Binary Classifier | Degree of expression of marker: CD27 |
| Binary Classifier | Degree of expression of marker: CD39 |
| Binary Classifier | Degree of expression of marker: CD4 |
| Binary Classifier | Degree of expression of marker: CD45RA |
| Binary Classifier | Degree of expression of marker: ICOS |
| Binary Classifier | Degree of expression of marker: PD-1 |
| Binary Classifier | Degree of expression of marker: Tregs |

TABLE 8C
Marker expression used to identify cell types for cells labeled with markers of panel 8.
Panel 8

| Markers | Cell Types |
|---|---|
| CD4+, Tregs+, CD45RA+, CD27+, CD39+, | CD4 Tregs CD45RA+ CD27+ CD39+ ICOS+ |
| ICOS+, PD-1+ | PD-1+ |
| CD4+, Tregs+, CD45RA+, CD27+, CD39+, | CD4 Tregs CD45RA+ CD27+ CD39+ ICOS+ |
| ICOS+, PD-1− | PD-1− |
| CD4+, Tregs+, CD45RA+, CD27+, CD39+, | CD4 Tregs CD45RA+ CD27+ CD39+ ICOS− |
| ICOS−, PD-1+ | PD-1+ |
| CD4+, Tregs+, CD45RA+, CD27+, CD39+, | CD4 Tregs CD45RA+ CD27+ CD39+ ICOS− |
| ICOS−, PD-1− | PD-1− |
| CD4+, Tregs+, CD45RA+, CD27+, CD39−, | CD4 Tregs CD45RA+ CD27+ CD39− ICOS+ |
| ICOS+, PD-1+ | PD-1+ |
| CD4+, Tregs+, CD45RA+, CD27+, CD39−, | CD4 Tregs CD45RA+ CD27+ CD39− ICOS+ |
| ICOS+, PD-1− | PD-1− |
| CD4+, Tregs+, CD45RA+, CD27+, CD39−, | CD4 Tregs CD45RA+ CD27+ CD39− ICOS− |
| ICOS−, PD-1+ | PD-1+ |
| CD4+, Tregs+, CD45RA+, CD27+, CD39−, | CD4 Tregs CD45RA+ CD27+ CD39− ICOS− |
| ICOS−, PD-1− | PD-1− |
| CD4+, Tregs+, CD45RA−, CD27−, CD39+, | CD4 Tregs CD45RA− CD27− CD39+ ICOS+ |
| ICOS+, PD-1+ | PD-1+ |
| CD4+, Tregs+, CD45RA−, CD27−, CD39+, | CD4 Tregs CD45RA− CD27− CD39+ ICOS− |
| ICOS−, PD-1+ | PD-1+ |
| CD4+, Tregs+, CD45RA−, CD27−, CD39−, | CD4 Tregs CD45RA− CD27− CD39− ICOS+ |
| ICOS+, PD-1+ | PD-1+ |
| CD4+, Tregs+, CD45RA−, CD27−, CD39−, | CD4 Tregs CD45RA− CD27− CD39− ICOS− |
| ICOS−, PD-1+ | PD-1+ |
| CD4+, Tregs+, CD45RA−, CD27−, CD39+, | CD4 Tregs CD45RA− CD27− CD39+ ICOS+ |
| ICOS+, PD-1− | PD-1− |
| CD4+, Tregs+, CD45RA−, CD27−, CD39+, | CD4 Tregs CD45RA− CD27− CD39+ ICOS− |
| ICOS−, PD-1− | PD-1− |
| CD4+, Tregs+, CD45RA−, CD27−, CD39−, | CD4 Tregs CD45RA− CD27− CD39− ICOS+ |
| ICOS+, PD-1− | PD-1− |
| CD4+, Tregs+, CD45RA−, CD27−, CD39−, | CD4 Tregs CD45RA− CD27− CD39− ICOS− |
| ICOS−, PD-1− | PD-1− |
| CD4+, Tregs+, CD45RA−, CD27+, CD39+, | CD4 Tregs CD45RA− CD27+ CD39+ ICOS+ |
| ICOS+, PD-1+ | PD-1+ |
| CD4+, Tregs+, CD45RA−, CD27+, CD39+, | CD4 Tregs CD45RA− CD27+ CD39+ ICOS− |
| ICOS−, PD-1+ | PD-1+ |
| CD4+, Tregs+, CD45RA−, CD27+, CD39−, | CD4 Tregs CD45RA− CD27+ CD39− ICOS+ |
| ICOS+, PD-1+ | PD-1+ |
| CD4+, Tregs+, CD45RA−, CD27+, CD39−, | CD4 Tregs CD45RA− CD27+ CD39− ICOS− |
| ICOS−, PD-1+ | PD-1+ |
| CD4+, Tregs+, CD45RA−, CD27+, CD39+, | CD4 Tregs CD45RA− CD27+ CD39+ ICOS+ |
| ICOS+, PD-1− | PD-1− |
| CD4+, Tregs+, CD45RA−, CD27+, CD39+, | CD4 Tregs CD45RA− CD27+ CD39+ ICOS− |
| ICOS−, PD-1− | PD-1− |
| CD4+, Tregs+, CD45RA−, CD27+, CD39−, | CD4 Tregs CD45RA− CD27+ CD39− ICOS+ |
| ICOS+, PD-1− | PD-1− |
| CD4+, Tregs+, CD45RA−, CD27+, CD39−, | CD4 Tregs CD45RA− CD27+ CD39− ICOS− |
| ICOS−, PD-1− | PD-1− |
| CD4+, Tregs+, CD45RA+, CD27−, CD39+, | CD4 Tregs CD45RA+ CD27− CD39+ ICOS+ |
| ICOS+, PD-1+ | PD-1+ |
| CD4+, Tregs+, CD45RA+, CD27−, CD39+, | CD4 Tregs CD45RA+ CD27− CD39+ ICOS− |
| ICOS−, PD-1+ | PD-1+ |
| CD4+, Tregs+, CD45RA+, CD27−, CD39−, | CD4 Tregs CD45RA+ CD27− CD39− ICOS+ |
| ICOS+, PD-1+ | PD-1+ |
| CD4+, Tregs+, CD45RA+, CD27−, CD39−, | CD4 Tregs CD45RA+ CD27− CD39− ICOS− |
| ICOS−, PD-1+ | PD-1+ |
| CD4+, Tregs+, CD45RA+, CD27−, CD39+, | CD4 Tregs CD45RA+ CD27− CD39+ ICOS+ |
| ICOS+, PD-1− | PD-1− |
| CD4+, Tregs+, CD45RA+, CD27−, CD39+, | CD4 Tregs CD45RA+ CD27− CD39+ ICOS− |
| ICOS−, PD-1− | PD-1− |
| CD4+, Tregs+, CD45RA+, CD27−, CD39−, | CD4 Tregs CD45RA+ CD27− CD39− ICOS+ |
| ICOS+, PD-1− | PD-1− |
| CD4+, Tregs+, CD45RA+, CD27−, CD39−, | CD4 Tregs CD45RA+ CD27− CD39− ICOS− |
| ICOS−, PD-1− | PD-1− |
| CD4+, Tregs−, CD45RA+, CD27+, CD39+, | CD4 CD45RA+ CD27+ CD39+ ICOS+ PD-1+ |
| ICOS+, PD-1+ | |
| CD4+, Tregs−, CD45RA+, CD27+, CD39+, | CD4 CD45RA+ CD27+ CD39+ ICOS+ PD-1− |
| ICOS+, PD-1− | |
| CD4+, Tregs−, CD45RA+, CD27+, CD39+, | CD4 CD45RA+ CD27+ CD39+ ICOS− PD-1+ |
| ICOS−, PD-1+ | |
| CD4+, Tregs−, CD45RA+, CD27+, CD39+, | CD4 CD45RA+ CD27+ CD39+ ICOS− PD-1− |
| ICOS−, PD-1− | |
| CD4+, Tregs−, CD45RA+, CD27+, CD39−, | CD4 CD45RA+ CD27+ CD39− ICOS+ PD-1+ |
| ICOS+, PD-1+ | |
| CD4+, Tregs−, CD45RA+, CD27+, CD39−, | CD4 CD45RA+ CD27+ CD39− ICOS+ PD-1− |
| ICOS+, PD-1− | |
| CD4+, Tregs−, CD45RA+, CD27+, CD39−, | CD4 CD45RA+ CD27+ CD39− ICOS− PD-1+ |
| ICOS−, PD-1+ | |
| CD4+, Tregs−, CD45RA+, CD27+, CD39−, | CD4 CD45RA+ CD27+ CD39− ICOS− PD-1− |
| ICOS−, PD-1− | |
| CD4+, Tregs−, CD45RA+, CD27−, CD39+, | CD4 CD45RA+ CD27− CD39+ ICOS+ PD-1+ |
| ICOS+, PD-1+ | |
| CD4+, Tregs−, CD45RA+, CD27−, CD39−, | CD4 CD45RA+ CD27− CD39− ICOS+ PD-1+ |
| ICOS+, PD-1+ | |
| CD4+, Tregs−, CD45RA+, CD27−, CD39+, | CD4 CD45RA+ CD27− CD39+ ICOS− PD-1+ |
| ICOS−, PD-1+ | |
| CD4+, Tregs−, CD45RA+, CD27−, CD39−, | CD4 CD45RA+ CD27− CD39− ICOS− PD-1+ |
| ICOS−, PD-1+ | |
| CD4+, Tregs−, CD45RA+, CD27−, CD39+, | CD4 CD45RA+ CD27− CD39+ ICOS+ PD-1− |
| ICOS+, PD-1− | |
| CD4+, Tregs−, CD45RA+, CD27−, CD39−, | CD4 CD45RA+ CD27− CD39− ICOS+ PD-1− |
| ICOS+, PD-1− | |
| CD4+, Tregs−, CD45RA+, CD27−, CD39+, | CD4 CD45RA+ CD27− CD39+ ICOS− PD-1− |
| ICOS−, PD-1− | |
| CD4+, Tregs−, CD45RA+, CD27−, CD39−, | CD4 CD45RA+ CD27− CD39− ICOS− PD-1− |
| ICOS−, PD-1− | |
| CD4+, Tregs−, CD45RA−, CD27+, CD39+, | CD4 CD45RA−CD27+ CD39+ ICOS+ PD-1+ |
| ICOS+, PD-1+ | |
| CD4+, Tregs−, CD45RA−, CD27+, CD39+, | CD4 CD45RA− CD27+ CD39+ ICOS+ PD-1− |
| ICOS+, PD-1− | |
| CD4+, Tregs−, CD45RA−, CD27+, CD39+, | CD4 CD45RA− CD27+ CD39+ ICOS− PD-1+ |
| ICOS−, PD-1+ | |
| CD4+, Tregs−, CD45RA−, CD27+, CD39+, | CD4 CD45RA− CD27+ CD39+ ICOS− PD-1− |
| ICOS−, PD-1− | |
| CD4+, Tregs−, CD45RA−, CD27+, CD39−, | CD4 CD45RA− CD27+ CD39− ICOS+ PD-1+ |
| ICOS+, PD-1+ | |
| CD4+, Tregs−, CD45RA−, CD27+, CD39−, | CD4 CD45RA− CD27+ CD39− ICOS+ PD-1− |
| ICOS+, PD-1− | |
| CD4+, Tregs−, CD45RA−, CD27+, CD39−, | CD4 CD45RA− CD27+ CD39− ICOS− PD-1+ |
| ICOS−, PD-1+ | |
| CD4+, Tregs−, CD45RA−, CD27+, CD39−, | CD4 CD45RA− CD27+ CD39− ICOS− PD-1− |
| ICOS−, PD-1− | |
| CD4+, Tregs−, CD45RA−, CD27−, CD39+, | CD4 CD45RA− CD27− CD39+ ICOS+ PD-1+ |
| ICOS+, PD-1+ | |
| CD4+, Tregs−, CD45RA−, CD27−, CD39+, | CD4 CD45RA− CD27− CD39+ ICOS+ PD-1− |
| ICOS+, PD-1− | |
| CD4+, Tregs−, CD45RA−, CD27−, CD39+, | CD4 CD45RA− CD27− CD39+ ICOS− PD-1+ |
| ICOS−, PD-1+ | |
| CD4+, Tregs−, CD45RA−, CD27−, CD39+, | CD4 CD45RA− CD27− CD39+ ICOS− PD-1− |
| ICOS−, PD-1− | |
| CD4+, Tregs−, CD45RA−, CD27−, CD39−, | CD4 CD45RA− CD27− CD39− ICOS+ PD-1+ |
| ICOS+, PD-1+ | |
| CD4+, Tregs−, CD45RA−, CD27−, CD39−, | CD4 CD45RA− CD27− CD39− ICOS+ PD-1− |
| ICOS+, PD-1− | |
| CD4+, Tregs−, CD45RA−, CD27−, CD39−, | CD4 CD45RA− CD27− CD39− ICOS− PD-1+ |
| ICOS−, PD-1+ | |
| CD4+, Tregs−, CD45RA−, CD27−, CD39−, | CD4 CD45RA− CD27− CD39− ICOS− PD-1− |
| ICOS−, PD-1− | |

TABLE 9
Example panel of markers and machine learning models used to predict
cell types for cells labeled with the panel of markers.
Panel 9

| | |
|---|---|
| Markers | CD45, NKp44, CD16, CD19, CD123, CD13, CD3, NKG2A, CD158, |
| | NKG2C, CD57, CD161, CD56, NKG2D, CD107a |
| Model Type | Trained to Predict |
| Multi-class | Cell types from among: |
| Classifier | NK cells CD56+ CD16− |
| | Immature NK cells |
| | Mature NK CD158+ CD57+ |
| | Mature NK CD158+ CD57− |
| | Mature NK CD158− CD57+ |
| | Mature NK CD158− CD57− |

TABLE 10A
Example panel of markers and machine learning models used to predict
cell types for cells labeled with the panel of markers.
Panel 10

| | |
|---|---|
| Markers | CD27, CD8, gdTCR, CD19, CD13, CD3, iNKT, TCR Vd2, CD57, |
| | CD161, CD56, TCR Va7.2, CD45RA |
| Model Type | Trained to Predict |
| Multi-class | Cell types from among: |
| Classifier | T Cells & Undefined |
| | MAIT CD8+ CD27+ CD56− CD57− |
| | MAIT CD8+ CD27+ CD56− CD57+ |
| | MAIT CD8+ CD27+ CD56+ CD57− |
| | MAIT CD8+ CD27+ CD56+ CD57+ |
| | MAIT CD8+ CD27− CD56− CD57+ |
| | MAIT CD8+ CD27− CD56− CD57− |
| | MAIT CD8+ CD27− CD56+ CD57− |
| | MAIT CD8− CD27+ CD56− CD57− |
| | MAIT CD8− CD27+ CD56− CD57+ |
| | MAIT CD8− CD27+ CD56+ CD57− |
| | gdT Vdelta2− CD56− CD57+ |
| | gdT Vdelta2− CD56− CD57− |
| | gdT Vdelta2− CD56+ CD57+ |
| | gdT Vdelta2− CD56+ CD57− |
| | gdT Vdelta2+ CD56+ CD57− |
| | gdT Vdelta2+ CD56+ CD57+ |
| | gdT Vdelta2+ CD56− CD57+ |
| | gdT Vdelta2+ CD56− CD57− |
| | iNKT |
| | Other T Cells |

TABLE 10B
Example panel of markers and machine learning models used to predict
cell types for cells labeled with the panel of markers.
Panel 10

| | |
|---|---|
| Markers | CD27, CD8, gdTCR, CD19, CD13, CD3, iNKT, TCR Vd2, CD57, |
| | CD161, CD56, TCR Va7.2, CD45RA |
| Artificial | MAIT (gdTCR, TCR−Va7.2, CD161), gdT (gdTCR, TCR−Vd2), iNKT |
| Markers | (gdTCR, TCR−Va24−Ja18) |
| Model Type | Trained To Predict |
| Binary Classifier | Degree of expression of marker: CD27 |
| Binary Classifier | Degree of expression of marker: CD56 |
| Binary Classifier | Degree of expression of marker: CD57 |
| Binary Classifier | Degree of expression of marker: CD8 |
| Binary Classifier | Degree of expression of marker: MAIT |
| Binary Classifier | Degree of expression of marker: gdT |
| Binary Classifier | Degree of expression of marker: iNKT |
| Binary Classifier | Degree of expression of marker: TCR Vd2 |

TABLE 10C
Marker expression used to identify cell types for cells labeled with markers of panel 10.
Panel 10

| Markers | Cell Types |
|---|---|
| gdT+, MAIT−, iNKT−, Vdelta2+, CD56−, CD57+ | gdT Vdelta2+ CD56− CD57+ |
| gdT+, MAIT−, iNKT−, Vdelta2+, CD56−, CD57− | gdT Vdelta2+ CD56− CD57− |
| gdT+, MAIT−, iNKT−, Vdelta2+, CD56+, CD57− | gdT Vdelta2+ CD56+ CD57− |
| gdT+, MAIT−, iNKT−, Vdelta2+, CD56+, CD57+ | gdT Vdelta2+ CD56+ CD57+ |
| gdT+, MAIT−, iNKT−, Vdelta2−, CD56+, CD57+ | gdT Vdelta2− CD56+ CD57+ |
| gdT+, MAIT−, iNKT−, Vdelta2−, CD56+, CD57− | gdT Vdelta2− CD56+ CD57− |
| gdT+, MAIT−, iNKT−, Vdelta2−, CD56−, CD57+ | gdT Vdelta2− CD56− CD57+ |
| gdT+, MAIT−, iNKT−, Vdelta2−, CD56−, CD57− | gdT Vdelta2− CD56− CD57− |
| gdT−, MAIT−, iNKT+ | iNKT |
| gdT−, MAIT+, iNKT−, CD8+, CD27+, CD56+, | MAIT CD8+ CD27+ CD56+ CD57+ |
| CD57+ | |
| gdT−, MAIT+, iNKT−, CD8+, CD27+, CD56+, | MAIT CD8+ CD27+ CD56+ CD57− |
| CD57− | |
| gdT−, MAIT+, iNKT−, CD8+, CD27+, CD56−, | MAIT CD8+ CD27+ CD56− CD57+ |
| CD57+ | |
| gdT−, MAIT+, iNKT−, CD8+, CD27+, CD56−, | MAIT CD8+ CD27+ CD56− CD57− |
| CD57− | |
| gdT−, MAIT+, iNKT−, CD8+, CD27−, CD56−, | MAIT CD8+ CD27− CD56− CD57+ |
| CD57+ | |
| gdT−, MAIT+, iNKT−, CD8+, CD27−, CD56−, | MAIT CD8+ CD27− CD56− CD57− |
| CD57− | |
| gdT−, MAIT+, iNKT−, CD8+, CD27−, CD56+, | MAIT CD8+ CD27− CD56+ CD57+ |
| CD57+ | |
| gdT−, MAIT+, iNKT−, CD8+, CD27−, CD56+, | MAIT CD8+ CD27− CD56+ CD57− |
| CD57− | |
| gdT−, MAIT+, iNKT−, CD8−, CD27+, CD56+, | MAIT CD8− CD27+ CD56+ CD57+ |
| CD57+ | |
| gdT−, MAIT+, iNKT−, CD8−, CD27+, CD56+, | MAIT CD8− CD27+ CD56+ CD57− |
| CD57− | |
| gdT−, MAIT+, iNKT−, CD8−, CD27+, CD56−, | MAIT CD8− CD27+ CD56− CD57+ |
| CD57+ | |
| gdT−, MAIT+, iNKT−, CD8−, CD27+, CD56−, | MAIT CD8− CD27+ CD56− CD57− |
| CD57− | |
| gdT−, MAIT+, iNKT−, CD8−, CD27−, CD56+, | MAIT CD8− CD27− CD56+ CD57+ |
| CD57+ | |
| gdT−, MAIT+, iNKT−, CD8−, CD27−, CD56+, | MAIT CD8− CD27− CD56+ CD57− |
| CD57− | |
| gdT−, MAIT+, iNKT−, CD8−, CD27−, CD56−, | MAIT CD8− CD27− CD56− CD57+ |
| CD57+ | |
| gdT−, MAIT+, iNKT−, CD8−, CD27−, CD56−, | MAIT CD8− CD27− CD56− CD57− |
| CD57− | |

TABLE 11
Artificial Markers.

| | Artificial | Relevant Markers | Marker Composition |
|---|---|---|---|
| Panel | Marker | from Panel | for Positive Value |
| 2 | Tregs | CD25, IL7RA | CD25+IL7RA− |
| 8 | Tregs | CD25, IL7RA | CD25+IL7RA− |
| 10 | MAIT | gdTCR, TCR−Va7.2, CD161 | TCRgd−Valpha7+CD161+ |
| 10 | gdT | gdTCR, TCR−Vd2 | TCRgd+Vdelta2+, TCRgd+Vdelta− |
| 10 | iNKT | gdTCR, TCR−Va24−Ja11 | TCRgd−, Va24−Ja18 TCR+ |

### Cell Composition Percentages and Applications

FIG. 4A is a flowchart of an illustrative process 400 for identifying a subject as a member of a patient cohort, according to some embodiments of the technology described herein. Process 400 may be performed in part or in full by a laptop computer, a desktop computer, one or more servers, in a cloud computing environment, computing device 108 as described herein with respect to FIG. 1D, computing device 1200 as described herein with respect to FIG. 12, or using any other suitable computing device(s), as aspects of the technology described herein are not limited in this respect.

Process 400 begins at act 402 for obtaining cytometry data for a biological sample from a subject, the biological sample including a plurality of cells. In some embodiments, act 402 may be performed according to the techniques described herein, including at least with respect to act 202 of process 200 and/or act 252 of process 250.

At act 404, a respective type is identified for each of at least some of the plurality of cells based on the cytometry data obtained at act 402. In some embodiments, act 404 may be performed according to the techniques described herein including at least with respect to FIGS. 2A-2B for identifying types for cells in a biological sample.

At act 406, a cell count is determined for each of multiple cell types identified at act 404. In some embodiments, this includes determining a number of cells, or cell count, of each type of cell for which cytometry measurements are obtained at act 402. The cell counts, in some embodiments, may be used to determine a number of cells of each type of cell included in at least a hierarchy of cell types. A hierarchy of cell types may indicate relationships between different cell types. For example, the hierarchy of cell types may include parent cell types and cell types that are children, or subtypes, of the parent cell type. Table 13 defines an example hierarchy of cell types. In some embodiments, data indicating a hierarchy of cell types is received as input at act 406. For example, a file of any suitable type (e.g., an Excel file) defining the hierarchy may be received as input.

In some embodiments, data indicating the types identified (at act 404) for each of multiple objects (e.g., cells, debris, beads, unidentified objects, etc.) in the biological sample may also be received at act 406. For example, the input may include a tab-separated values file having a number of lines corresponding to the number of objects. Each of at least some of the lines may include an indication of the type determined for the object. In some embodiments, at least some of the cell types indicated for the objects are included in the hierarchy of cell types. In some embodiments, one or more cell types are not included in the hierarchy of cell types. For example, the identified cell types may include types for “doubles,” which are a combination of two different cell types (e.g., “Monocytes & Neutrophils”). As another example, the identified cell types may include one or more custom cell types which one or more of the machine learning models described herein were trained to predict (e.g., “Dead Neutrophils”).

In some embodiments, a “raw” cell count is determined for each unique cell type listed in the data indicating the types identified for the biological sample. For example, this includes determining counts for types that are included in the hierarchy of cell types and types that are not included in the hierarchy of cell types.

In some embodiments, the determined cell counts are then updated to conform with cell types included in the hierarchy of cell types. For example, this may include attributing a cell count determined for an identified cell type that is not included in the hierarchy to a cell type that is included in the hierarchy. For example, a cell count determined for the identified cell type of “Dead Neutrophils,” which is not included in the hierarchy, may be attributed to the cell type “Neutrophils,” which is included in the hierarchy. For example, the cell count may be added to the cell count for neutrophils. Accordingly, in some embodiments, since the cell count is accounted for by the “Neutrophil” cell type, the cell count for “Dead Neutrophils” may be discarded. In some embodiments, in updating the determined cell counts to conform with cell types included in the hierarchy of cell types, “doubles” may also be split into two different cell types, and cell counts may be updated for the respective cell types accordingly. For example, a count of “Monocytes & Neutrophils”) may be split into a count of Monocytes and a count of Neutrophils. Accordingly, in some embodiments, any existing cell counts for Monocytes and Neutrophils may be updated to include said counts. Since the cell counts are accounted for by the “Monocyte” and “Neutrophil” cell type, the cell count for “Monocyte & Neutrophil” may be discarded.

In some embodiments, cell counts for parent cell types in the hierarchy of cell types are determined as a sum of the cell counts of their descendants (e.g., subtypes). For example, a cell that is identified to be a “Classical Monocyte” is also a “Monocyte,” since “Classical Monocyte” is a subtype of “Monocyte.” Accordingly, in some embodiments, the cell count of a parent cell type in the hierarchy of cell types may be updated based on the cell counts of its descendants. For example, the cell counts of the descendants may be added to an existing cell count for the parent or added from zero, if there is no existing cell count for the parent cell type. In some embodiments, the techniques for updating cell counts of parent cell types may be carried out sequentially from the bottom of the hierarchy of cell types to the top of the hierarchy of cell types.

At act 408, a cell composition percentage is determined for each of at least some of the identified cell types. In some embodiments, determining a cell composition percentage for a particular cell type includes determining a ratio between the number of cells of a particular type and a total number of cells determined for the biological sample. For example, the total number of cells may be determined as the number of leukocytes determined for the biological sample.

In some embodiments, the cell composition percentages determined for particular cell types are used to determine cell concentrations of those cell types in the biological sample. For example, the normalized cell composition percentages may be multiplied by a respective coefficient that converts the cell composition percentage to a cell concentration.

In some embodiments, coefficients for converting a cell composition percentage to a cell concentration are determined based on measurements obtained using a device configured to measure a concentration of one or more cell types in the biological sample. The device may include any suitable device configured to obtain such measurements such as, for example, a hematology analyzer, as aspects of the technology described herein are not limited in this respect. For example, in some embodiments, the device may be configured to measure the concentration of lymphocytes, the concentration of monocytes, and the concentration of the combination of neutrophils, basophils, and eosinophils.

In some embodiments, the coefficient is determined based on a ratio of the measured concentration of a particular cell type in the biological and a corresponding cell composition percentage. For example, a first coefficient may include the ratio of the measured concentration of lymphocytes and the cell composition percentage determined for lymphocytes. A second coefficient may include a ratio of the measured concentration of monocytes and the sum of the cell composition percentages determined for monocytes and dendritic cells. A third coefficient may include a ratio of the measured concentration of neutrophils, basophils, and eosinophils and the cell composition percentage determined for granulocytes.

In some embodiments, a particular coefficient may be applied to cell composition percentages determined for the cell type(s) used to determine the particular coefficient, and the descendants (e.g., subtypes) of those cell type(s). For example, the first coefficient may be applied (e.g., used to multiply) to the cell composition percentage determined for lymphocytes and the cell composition percentages determined for descendants of lymphocytes. The second coefficient may be applied to the cell composition percentages determined for monocytes and dendritic cells and the cell composition percentages determined for descendants of monocytes and dendritic cells. The third coefficient may be applied to the cell composition percentage determined for granulocytes and the cell composition percentages determined for descendants of granulocytes.

Act 410 includes identifying the subject as a member of a patient cohort based on the determined cell composition percentages and/or cell concentrations. In some embodiments, this may include comparing one or more cell composition percentages to those associated with a patient cohort. As a nonlimiting example, this may include comparing the percentage of a subject's T cells to the average percentage of T cells in patients who responded positively to a particular treatment. Additionally, or alternatively, this may include comparing one or more cell concentrations to those associated with a patient cohort. FIGS. 6A-6D show example reports comparing cell composition percentages and cell concentrations determined for a biological sample to ranges of cell composition percentages and cell concentrations of a healthy cohort.

In some embodiments, identifying a subject as a member of a cohort may be useful in making diagnoses, developing treatment plans, identifying effective drugs, and conducting research. However, it should be appreciated that this is a non-exhaustive list.

Act 412 includes identifying a treatment for the subject based on the determined cell composition percentages and/or cell concentrations. The determined cell composition percentages may serve as biomarkers that can be used to identify treatments for the subject. For example, the cell composition percentage of peripheral blood mononuclear cells (PBMC) may serve as a biomarker for identifying an antibody anti-cancer agent, such as ipilimumab as a treatment for subjects with HLA-DRlow Monocytes. In some embodiments, if the determined cell composition percentage of PBMCs for the subject is below a threshold value, then the antibody anti-cancer agent may be identified as a treatment for the subject. For example, if the cell composition percentage of PBMCs is below a threshold value of 10%, 11%, 12%, 13%, 13.05%, 13.1%, 13.5%, 14%, 15%, 16%, or a threshold value between 10% and 16%, then the antibody anti-cancer agent may be identified as a treatment for the subject. As another example, the cell composition percentages of CD8+PD-1+ cells and CD4+PD-1+ cells may serve as biomarkers for identifying immune checkpoint blockade therapy for a subject with non-small cell lung cancer (NSCLC). For example, the ratio of the cell composition percentage of CD8+PD-1+ cells to CD4+PD-1+ cells may serve as such a biomarker. In some embodiments, if the ratio exceeds a threshold value, then immune checkpoint blockade therapy is identified as a treatment for the subject. For example, if the ratio exceeds a threshold value of 1.5, 1.6, 1.7, 1.8, 1.85, 1.89, 1.9, 1.91, 1.92, 1.93, 1.95, 2.0, 2.1, 2.2, 2.3, or a threshold value between 1.5 and 2.3, then immune checkpoint blockade therapy may be identified as a treatment for the subject.

Act 414 includes administering, to the subject, the treatment identified at act 412. Techniques for administering the treatment are described herein including at least in the “Methods of Treatment” section.

It should be appreciated that one or more acts included in process 400 are optional and may be omitted. For example, act 414 may be omitted, acts 412-414 may be omitted, act 410 may be omitted, acts 410 and act 412-414 may be omitted, or any other suitable acts in process 400 may be omitted.

As described above, in some embodiments, cells in the same biological sample may be split into two or more subgroups, termed “subsamples,” and labeled with different panels of markers. The different panels of markers may have some markers in common but differ in at least one or more markers. Obtaining cytometry measurements for different markers allows for the identification of different cell types. For example, one panel of markers may be used to distinguish naive B cells from other cell types in a subsample, while a different panel of markers may be used to distinguish monocytes from other cell types in a different subsample.

In some embodiments, cytometry data obtained for a particular subsample may be used to estimate cell counts for the cell types identified in that particular subsample. For example, for cells identified as T cells in a first subsample, a cell count for T cells may be estimated. However, due to variability among the different subsamples, percentage of T cells in the first subsample may not (and likely does not) accurately reflect the percentage of T cells in the overall biological sample. Accordingly, the inventors have developed techniques for normalizing cell counts determined for different subsamples, such that they are invariant to the different subsamples. In some embodiments, the normalized cell counts may then be used to determine cell composition percentages for cell types in the biological sample.

FIG. 4B is a flowchart of an illustrative process 420 for determining cell composition percentages based on cell counts determined for different subsamples of a biological sample, according to some embodiments of the technology described herein. Process 420 may be performed in part or in full by a laptop computer, a desktop computer, one or more servers, in a cloud computing environment, computing device 108 as described herein with respect to FIG. 1D, computing device 1200 as described herein with respect to FIG. 12, or using any other suitable computing device(s), as aspects of the technology described herein are not limited in this respect. In some embodiments, process 420 is an example implementation of act 408 of process 400 in FIG. 4A.

In some embodiments, process 420 includes one or more acts that may performed independently of one another. For example, acts 422-1, 424-1, and 426-1 may be performed independently of one or more of acts 422-2, 424-2, and 426-2. Such acts may be performed sequentially or in parallel.

At act 422-1, first cytometry data is obtained for a first subsample of a biological sample. In some embodiments, act 422-1 may be performed according to the techniques described herein, including at least with respect to act 202 of process 200 and/or act 252 of process 250. In some embodiments, the first cytometry data includes cytometry measurements that were obtained using a first panel of markers. The cytometry measurements may include measurements for at least some of a first plurality of cells included in the first subsample.

At act 422-2, second cytometry data is obtained for a second subsample of a biological sample. In some embodiments, act 422-2 may be performed according to the techniques described herein, including at least with respect to act 202 of process 200 and/or act 252 of process 250. In some embodiments, the second cytometry data includes cytometry measurements that were obtained using a second panel of markers. For example, the second panel of markers may include one or more markers that are different from the markers included in the first panel. The cytometry measurements may include measurements for at least some of a second plurality of cells included in the second subsample.

At act 424-1, the first cytometry data is used to identify a first plurality of cell types for the first plurality of cells included in the first subsample. In some embodiments, act 424-1 may be performed according to the techniques described herein including at least with respect to FIGS. 2A-2B for identifying types for cells in a biological sample.

At act 424-2, the second cytometry data is used to identify a second plurality of cell types for the second plurality of cells included in the second subsample. In some embodiments, act 424-2 may be performed according to the techniques described herein including at least with respect to FIGS. 2A-2B for identifying types for cells in a biological sample.

At act 426-1, cell counts are determined for at least some of the first plurality of cell types identified at act 424-1. In some embodiments, act 426-1 may be performed according to the techniques described herein including at least with respect to act 406 of process 400 in FIG. 4A. For example, this may include determining a “raw” cell count for the identified cell types.

Additionally, or alternatively, this may include determining cell counts for cell types included in a hierarchy of cell types.

At act 426-2, cell counts are determined for at least some of the second plurality of cell types identified at act 424-2. In some embodiments, act 426-1 may be performed according to the techniques described herein including at least with respect to act 406 of process 400 in FIG. 4A. For example, this may include determining a “raw” cell count for the identified cell types.

Additionally, or alternatively, this may include determining cell counts for cell types included in a hierarchy of cell types.

At act 428, the cell counts determined for the first plurality of cell types and/or the second plurality of cell types are normalized. In some embodiments, one or more normalization techniques may be used to normalize the cell counts. In some embodiments, one such technique includes normalizing the cell counts with respect to cell counts determined for a subsample associated with a “leader panel.” In some embodiments, a subsample is associated with a leader panel when types are identified for cells in the subsample based on cytometry measurements obtained using the leader panel. In some embodiments, a leader panel may include a panel of markers used to obtain cytometry measurements that can be used to distinguish among particular cell types. For example, the particular cell types may include one or more cell types that are common between the leader panel and a non-leader panel. In other words, the non-leader panel and the leader panel may each be used to obtain cytometry data that can be used to identify the common cell type. For example, Table 12 lists cell types that are common between non-leader panels and panel 3 in Table 3, which is an example of a leader panel.

TABLE 12
Cell types common between non-leader panels and a leader panel.

| Panel | Reference Cell Population |
|---|---|
| 1 | T cells |
| 2 | T cells |
| 4 | PBMC with Basophils |
| 5 | B cells |
| 6 | PBMC with Basophils |
| 7 | T cells |
| 8 | T cells |
| 9 | NK cells |
| 10 | T cells |

In some embodiments, normalizing cell counts using a leader panel includes determining a normalization coefficient that may be used to normalize cell counts determined for subsamples associated with non-leader panels. For example, in some embodiments, the normalization coefficient may be a ratio of (a) a number of cells of a reference type included in a subsample associated with a non-leader panel and (b) a number of cells of the reference type included in the subsample associated with the leader panel. In some embodiments, the reference type is a common cell type between the two panels.

In some embodiments, normalizing the cell counts includes normalizing the cell counts determined for the subsample associated with the non-leader panel by multiplying those cell counts by the normalization coefficient. For example, determining a normalized cell count, Count_{Normalized,i}, for a cell type i may be calculated using Equation 2:

$${Count}_{Normalized} = {Count}_{Unnormalized} * \frac{{Count}_{ref , leader}}{{Count}_{ref , non - leader}} \qquad ( Equation 2 )$$

In some embodiments, an additional, or alternative, normalization technique includes normalizing cell counts with respect to beads in a subsample. As described herein, a known concentration of beads may be added to the biological sample. Accordingly, the number of beads identified in a subsample may be used to normalize the number of cells of different types in the subsample. In some embodiments, the number of beads may be determined based on the number of events for which the identified event type indicates that the particular event corresponds to a bead being measured by the biological platform. In some embodiments, a normalized cell count Count_{Normalized,i}, for a cell type i is determined using Equation 3:

$${Count}_{Normalized , i} = {Concentration}_{B e a d s} * \frac{{Count}_{Unnormalized , i}}{{Count}_{beads}} \qquad ( Equation 3 )$$

where Concentration_{Beads} is the concentration of beads added to the biological sample.

As one nonlimiting example, the concentration of beads added to the biological sample may be 5,000 beads per million cells.

In some embodiments, after normalizing cell counts for each of at least some of the first plurality of cells and the second plurality of cells, a cell composition percentage is determined, at act 430, for each of at least some cell types of the first plurality of cell types and the second plurality of cell types. In some embodiments, for a cell type that is not shared by the first plurality of cells and the second plurality of cells, the cell composition percentage CCPᵢ for the cell type i may be determined using Equation 4:

$${CCP}_{i} = ( \frac{{Count}_{Normalized , i , n}}{{Count}_{T o t a l}} ) * 100 \% \qquad ( Equation 4 )$$

where Count_{Normalized,i,n} is the number of cells of type i included in the plurality of cells of subsample n (e.g., the first subsample, the second subsample, etc.) and where Count_{Total} is the total number of cells determined for the subsample n. In some embodiments, if any of the subsamples are associated with a leader panel, then Count_{Total} may be the total number of cells determined for the subsample associated with the leader panel. If none of the subsamples are associated with the leader panel, then Count_{Total} may be the total number of cells determined for the subsample n including the plurality of cells. As an example, the total number of cells may be based on the number of leukocytes determined for a particular subsample.

In some embodiments, for a cell type that is shared by the first plurality of cells and the second plurality of cells, determining the cell composition percentage CCPᵢ for the cell type i may depend on whether the first subsample or the second subsample is associated with a leader panel. In some embodiments, if any of the subsamples (e.g., the first subsample, the second subsample) are associated with a leader panel, that subsample is taken to be the reference subsample and the cell composition percentage may be determined using Equation 5:

$${CCP}_{i} = ( \frac{{Count}_{Normalized , i , r e f}}{{Count}_{Total , ref}} ) * 100 \% \qquad ( Equation 5 )$$

where Count_{Normalized,i,ref} is the number of cells of type i included in the reference plurality of cells and Count_{Total,ref} is the total number of cells determined for the reference subsample. As an example, the total number of cells may be based on the number of leukocytes determined for the reference subsample.

In some embodiments, if none of the subsamples are associated with a leader panel, then the cell composition percentage for the shared cell type may be determined by averaging cell composition percentages determined for the shared cell type. For example, if both the first subsample and the second subsample include a cell identified as being of a first cell type, and neither the first subsample nor the second subsample are associated with a leader panel, then a cell composition percentage for the first cell type may be determined for each subsample, and the determined cell composition percentages averaged. For example, the cell composition percentages may be determined for a particular cell type i using Equation 4, where Count_{Total} is the total number of cells determined for the particular subsample n. After calculating the cell composition percentages for the particular cell type, they may be averaged.

At act 432, the determined cell composition percentages are normalized with respect to hierarchical relationships between cell types. As described above, some techniques include determining cell composition percentages for different levels of a hierarchy of cell types. The cell composition percentage of a more general cell type (e.g., a “parent” cell type) should, in theory, be equivalent to the sum of its “descendant” cell types. For example, B cells, T cells, and NK cells are subtypes of lymphocytes. Therefore, the sum of the cell composition percentages for these types should be equal to the cell composition percentage determined for lymphocytes.

However, in some embodiments, the sum of the estimated cell composition percentages of descendant cell types may exceed the estimated cell composition percentage of the parent cell type. This may occur as a result of normalizing the cell counts at act 408. For example, when a parent cell type and children cell types (subtype) are identified using different panels, normalization may not fully account for the relationship between them. Therefore, it is possible that the sum of the normalized cell counts determined for the children cell types exceeds that of the parent cell type. Accordingly, in some embodiments, the techniques described herein include normalizing the estimate cell composition percentages, such that the sum of the estimate cell composition percentages of the descendant cell types do not exceed the cell composition percentage of the parent cell type. Techniques for normalizing the cell composition percentages with respect to hierarchical relationships between cell types are described herein including at least with respect to FIG. 4C.

It should be appreciated that, while process 420 includes acts for determining cell composition percentages based on cytometry data obtained for a first subsample and a second subsample, the techniques described herein for determining cell composition percentages are not limited to any particular number of subsamples. For example, the techniques described herein may be used to determine cell composition percentages based on cytometry data obtained for one subsample, at least 5 subsamples, at least 10 subsamples, at least 20 subsamples, at least 50 subsamples, between 1 and 100 subsamples, or any other suitable number of subsamples, as aspects of the technology described herein are not limited in this respect.

FIG. 4C is a flowchart of an illustrative process 460 for normalizing cell composition percentages with respect to hierarchical relationships between cell types, according to some embodiments of the technology described herein. Process 420 may be performed in part or in full by a laptop computer, a desktop computer, one or more servers, in a cloud computing environment, computing device 108 as described herein with respect to FIG. 1D, computing device 1200 as described herein with respect to FIG. 12, or using any other suitable computing device(s), as aspects of the technology described herein are not limited in this respect. In some embodiments, process 460 is an example implementation of act 408 of process 400 in FIG. 4A.

In some embodiments, there may be challenges associated with normalizing cell composition percentages with respect to hierarchical relationships between cell types. In particular, such challenges may arise when determining cell composition percentages based on data from multiple different subsamples. Consider, for example, a first subsample including cells of Type A1 and Type A2 and a second subsample including cells of Type A3 and Type A4, where each of cell Types A1, A2, A3, and A4 are subtypes of Type A. For the first subsample, the cell composition percentage of Type A should be equivalent to the sum of the cell composition percentage of Type A1 and the cell composition percentage of Type A2 (e.g., Type A=Type A1+Type A2). For the second subsample, the cell composition percentage of Type A should be equivalent to the sum of the cell composition percentage of Type A3 and the cell composition percentage of Type A4 (e.g., Type A=Type A3+Type A4). However, when combining data from different subsamples to determine cell composition percentages for the biological sample, the combined cell composition percentage of Type A is not equivalent to sum of the cell composition percentages of Types A1, A2, A3, and A4 (e.g., Type A Type A1+Type A2+Type A3+Type A4). Therefore, the subtypes of different subsamples may be treated independently from one another when normalizing according to the techniques described with respect to process 460.

At act 462, sets of one or more subtypes of a first cell type are identified for which one or more cell composition percentages have been estimated. When cell composition percentages were estimated from cytometry data obtained using two or more panels of markers, corresponding to two or more subsamples, there may be multiple respective sets of cell subtypes. For example, for a leukocyte, a first set may include monocytes and macrophages, while a second set may include lymphocytes and granulocytes.

At act 464, for a first set of the identified sets, a sum of the cell composition percentages estimated for subtypes included in the first set is determined. For example, for a first cell type of leukocytes having a first set of subtypes including monocytes and macrophages. The cell composition percentages estimated for monocytes and macrophages may be summed at act 464.

In some embodiments, at act 466, the sum is compared to the cell composition percentage estimated for the first cell type. For example, the sum of the cell composition percentages of monocytes and macrophages may be compared to the cell composition percentage of leukocytes.

If the sum of the cell composition percentages estimated for the subtypes is determined to exceed the cell composition percentage estimated for the first cell type, then process 460 proceeds to act 470. If the sum of the cell composition percentages estimated for the subtypes is determined not to exceed the cell composition estimated for the first cell type, then process 460 proceeds to act 468.

At act 468, the process 460 includes determining whether there are additional subtypes of the first cell type that are not included in the sets of one or more subtypes identified at act 462. For example, subtypes that are not included in identified sets may include subtypes included in a hierarchy of cell types (e.g., the hierarchy of cell types in Table 13), but for which cell composition percentages were not estimated.

If, at act 468, it is determined that there are additional subtypes, then no normalization coefficient is determined for the cell subtypes, or a normalization coefficient of 1 is determined for the cell subtypes. In either case, the cell composition percentages estimated for the cell subtypes may remain the same. In such an embodiment, process 460 may proceed to act 476.

If, at act 468, it is determined that there are no additional subtypes, then process 460 proceeds to act 470.

At act 470, in some embodiments, a normalization coefficient is determined for subtypes of the first cell type. In some embodiments, determining the normalization coefficient includes determining a ratio between the cell composition percentage estimated for the first cell type and the sum of the cell composition percentages estimated for the subtypes included in the first set of the identified sets. Additionally, or alternatively, a normalization coefficient may be determined in any suitable way, as aspects of the technology are not limited to any particular technique for determining a normalization coefficient.

At act 472, the determined normalization coefficient is applied to cell composition percentages estimated for one or more (e.g., one, some, or all) of the subtypes included in the identified set of subtypes. For example, this may include multiplying a cell composition percentage by the normalization coefficient.

At act 474, process 460 includes determining whether there is another set (e.g., a second set) in the identified sets of subtypes of the first cell type. If there is another set, then one or more of acts 464, 466, 468,470, and 472 may be repeated for the next set of the identified sets. If the identified sets is determined not to include another set, process 460 proceeds to act 476.

At act 476, the techniques include determining whether there are other cell types (e.g., a second cell type) for which the normalization techniques described herein may be applied. If there is another cell type, the one or more of acts 464, 464, 466, 468, 470, 472, and 474 may be repeated for the second cell type.

The inventors have recognized that cell types can be divided into various different subtypes based on the expression of particular combinations of markers. For example, the T-cell population can be divided into: (i) CD27+ T-cells and CD27− cells and (ii) TIGIT+ T-cells and TIGIT− T-cells, among others. Therefore, in some embodiments, cell composition percentages are determined based on the particular combination of markers (e.g., “marker composition”) expressed by cells. For example, the specific combination of markers expressed by a cell may be determined using the marker machine learning models described herein.

FIG. 4D is a flowchart of an illustrative process 480 for determining cell composition percentages based on marker composition, according to some embodiments of the technology described herein. Process 480 may be performed in part or in full by a laptop computer, a desktop computer, one or more servers, in a cloud computing environment, computing device 108 as described herein with respect to FIG. 1D, computing device 1200 as described herein with respect to FIG. 12, or using any other suitable computing device(s), as aspects of the technology described herein are not limited in this respect. In some embodiments, process 480 is an example implementation of act 408 of process 400 in FIG. 4A.

At act 481, cytometry data is obtained for a subsample using a panel of markers, the subsample including a plurality of cells. Techniques for obtaining cytometry data for a subsample are described herein including at least with respect to act 202 of process 200 shown in FIG. 2A and act 252 of process 250 shown in FIG. 2B. In some embodiments, the cytometry data includes cytometry measurements that were obtained using a panel of markers. The cytometry measurements may include measurements for a plurality of cells in the subsample.

At act 482, a plurality of marker compositions are identified for the plurality of cells. In some embodiments, determining a marker composition for a particular cell includes using one or more machine learning models to predict which markers are expressed and which markers are not expressed in a particular cell. For example, if Panel 10 is used to obtain the cytometry data, then the machine learning model trained to predict the degree to which CD27 is expressed (e.g., the binary model shown in Table 10B) may be used to predict whether CD27 is expressed (e.g., CD27+) or not expressed (e.g., CD27−) in each of one or more (e.g., all) of the cells in the subsample. This may be repeated for any suitable number of markers. Continuing with the example of cytometry data obtained using Panel 10, for each particular cell of at least some (e.g., all) of the cells in the subsample, one, some, or all of the machine learning models listed in Table 10B may be used to predict whether corresponding markers are expressed in the particular cell. Accordingly, a respective cell composition may be obtained for each of at least some of the cells in the subsample. Additional examples of panels and corresponding machine learning models that can be used to determine marker composition are shown in Table 1A, Table 2B, Table 7A, and Table 8B.

At act 483, the marker compositions are used to identify cell types for at least some of the plurality of cells. As described herein, the presence or absence of a particular combination of markers (e.g., as determined using marker machine learning models described herein), may be used to determine one or more cell types. Example marker compositions and corresponding cell types are listed in Table 1B, Table 2C, Table 7B, and Table 8B. Example techniques for determining cell types based on marker composition are described herein including at least with respect to act 206-4 of process 200 shown in FIG. 2A and act 256-5 of process 250 shown in FIG. 2B.

At act 484, counts are determined for at least some of the marker compositions including a first count for the first marker composition. In some embodiments, this includes determining a count for each unique marker composition determined at act 482. For example, for a particular marker composition CD3+PD1+, determining the count for the marker composition would include determining the number of cells for which the combination of CD3+ and PD1+ was determined at act 482.

At act 485, a parent cell type of the first marker composition is identified. In some embodiments, identifying a parent cell type includes identifying a cell type that can be further divided into subpopulations based on marker composition. In some embodiments, this is performed by identifying parent cell types using Tables 1B, 2C, 7B, and 10C. For example, the cell types listed in Tables 1B, 2C, 7B, and 10C follow the naming format: [Cell Type; Markers](e.g., CD8 Central Memory CD39+PD-1+ TIGIT+ Tim-3−, etc.). Specifically, in this example, MAIT cells are the parent cell type of the marker composition: CD8+, CD27+, CD45RA−, CD62L+, CD39+, PD-1+, TIGIT+, Tim-3−.

At act 486, a cell count is determined for a parent cell type of first marker composition, the parent cell type being one of the cell types identified for the cells at act 485. In some embodiments, determining the cell count includes determining the number of cells identified as being of the parent cell type at act 483.

At act 487, a fraction is determined between the first count for the first marker composition and the cell count for the parent cell type. In some embodiments, this includes summing the number of cells in the sample determined to be of the parent cell type. In some embodiments, this includes determining the fraction (Fractionᵢ) shown in Equation 6:

$${Fraction}_{i} = \frac{\# Cells Having Marker Composition i}{\# Cells of Parent Cell Type of Marker Composition i} \qquad ( Equation 6 )$$

At act 488, process 480 includes determining whether there is another marker composition for which a cell composition percentage is to be determined. In some embodiments, this includes determining whether there was another marker composition identified at act 482. If there is another marker composition (e.g., a second marker composition), one or more of acts 485, 486, and 487 may be repeated for the second marker composition. For example, a parent cell type may be identified for the second marker composition, a second cell count may be determined for the second marker composition, and a fraction may be determined between second count and the cell count for the respective parent cell type (e.g., using Equation 6).

If it is determined that there are no additional marker compositions at act 488, then process 480 proceeds to act 489. At act 489, process 480 includes determining whether a parent marker composition exists for one or more (e.g., all) of the marker composition(s) identified at act 482. In some embodiments, a parent marker composition for a particular marker composition refers to a marker composition having markers that are all shared by the particular marker composition. For example, M1+M2− would be the parent marker composition of M1+M2− M3+ because all the markers (e.g., M1+M2−) in the parent marker composition are shared by the child marker composition. In some embodiments, the parent-child relationship only exists for marker compositions obtained for the same subsample.

If it is determined, at act 489, that there are parent marker composition(s) for one or more of the marker compositions identified at act 482, then process 480 proceeds to act 490. Otherwise, process 480 proceeds to act 491.

At act 490, the fraction(s) determined at act 487 are normalized with respect to the corresponding fraction(s) determined for the parent marker composition(s). For example, if a parent marker composition was identified for the first marker composition at act 489, then act 490 may include normalizing the fraction of the first marker composition with respect to the fraction of the parent marker composition.

At act 491, process 480 includes determining whether there is cytometry data for another subsample. For example, this may include determining whether there is second cytometry data for a second subsample (e.g., obtained using a different panel). If there is cytometry data for a second subsample, process 480 may include repeating one or more of acts 481-490 for the second subsample. If it is determined that there are no additional subsamples, then process 480 proceeds to act 492.

At act 492, cell composition percentage(s) are determined for one or more (e.g., all) of the parent cell types identified at act 485. For example, a cell composition percentage may be determined for cells of the parent cell type identified for the first marker composition. In some embodiments, the cell composition percentage(s) are determined using the cell count(s) determined for the parent cell type(s). Techniques for determining cell composition percentages for cell types using a single or multiple panels are described herein including at least with respect to FIGS. 4A-4C and FIGS. 5A-5D.

At act 493, cell composition percentage(s) are determined for cells having the marker composition(s) identified at act 482. For example, a cell composition percentage may be determined for cells having the first marker composition. In some embodiments, determining the cell composition percentage(s) includes using the fraction(s) determined at act 487 and/or the normalized fraction(s) determined at act 490 (if applicable). The approach may vary depending on whether the marker composition was identified for a single subsample or for multiple subsamples (thus resulting in multiple fraction(s) for the same marker composition). In some embodiments, if the marker composition was identified for a single subsample, then determining the cell composition percentage for cells having that particular marker composition includes multiplying the fraction (or normalized fraction) determined for the particular marker composition (e.g., Equation 7) by the cell composition percentage determined for the parent cell type identified for the particular marker composition. In some embodiments, if the marker composition was identified for multiple subsamples, then determining the cell composition percentage for cells having that particular marker composition includes determining an average of the fractions (or normalized fractions) determined for the particular marker composition, then multiplying the average fraction by the cell composition percentage determined for the parent cell type identified for the particular marker composition (e.g., Equation 8).

$${CCP}_{i} = Avg . {Fraction}_{i} \times {CCP}_{Parent Cell Type , i} \qquad ( Equation 7 )$$

$${CCP}_{i} = Avg . {Fraction}_{i} \times {CCP}_{Parent Cell Type , i} \qquad ( Equation 8 )$$

Examples for determining cell composition percentages for marker compositions are described herein including at least with respect to FIG. 5E.

FIG. 5A depicts an illustrative example for determining cell composition percentages based on cell types determined using cytometry data obtained by performing cytometry using a single panel of markers, according to some embodiments of the technology described herein.

As shown in the example of FIG. 5A, cell types 502 were identified for five cells. Three different cell types (A, B, and C) were identified for the five cells. Accordingly, cell composition percentages 504 may be determined for the three cell types.

In some embodiments, determining the cell composition percentage each cell type includes determining a ratio of the number of cells identified as being of that type and the total number of cells. For example, determining the cell composition percentage of cell type A may include determining the ratio of the number of cells of type A (e.g., 2) and the total of number of cells (e.g., 5). In this example, the ratio is 2/5, resulting in a cell composition percentage of 40%. Determining a cell composition percentage of cell type B may include determining the ratio of the number of cells of type B (e.g., 2) and the total number of cells (e.g., 5). In this example, the ratio is 2/5, resulting in a cell composition percentage of 40%. Determining a cell composition percentage of cell type C may include determining the ratio of the number of cells of type C (e.g., 1) and the total number of cells (e.g., 5). In this example, the ratio is 1/5, resulting in a cell composition percentage of 20%.

FIG. 5B depicts an illustrative example for determining cell composition percentages based on cell types determined using cytometry data obtained by performing cytometry using different panels, where one of the panels is a leader panel, according to some embodiments of the technology described herein.

As shown, cell types 506 were identified using cytometry measurements for markers in a leader panel and cell types 510 were identified using cytometry measurements for markers in a non-leader panel. Cell types 506 and cell types 510 each include cell types A and E. Cell types 506 include cell type B, and cell types 510 include cell type E. In some embodiments, techniques described herein may be used to determine a single cell composition percentage for each cell type.

In some embodiments, the techniques include determining, for cell types associated with the leader panel, the number 508 of each cell type is determined. For example, the number of cells of cell type A (e.g., #cell type A1) is 2, the number of cells of cell type B (e.g., #cell type B1) is 2, and the number of cells of cell type E (e.g., #cells type E1) is 1.

In some embodiments, the techniques include determining a normalized cell count for each of the cell types associated with the non-leader panel 510. For example, this may first include determining an unnormalized cell count for each cell type. For example, the number of cells of cell type A (e.g., #cell type A2) is 3, the number of cells of cell type D (e.g., #cell type D2) is 1, and the number of cells of cell type E (e.g., #cells type E2) is 1.

In some embodiments, cells counts of a cell type shared by cell types 506 and 510 is used to obtain normalized cell counts 512. For example, since A is a shared cell type, the cell count for cell type A determined for cell types 506 (e.g., 2 cells) and the cell count for cell type A determined for cell types 510 (e.g., 3 cells) may be used to determine normalization coefficient. For example, the normalization coefficient may be the ratio of 2/3.

In some embodiments, the normalization coefficient is used to determined normalized cell counts 512. For example, each of the unnormalized cell counts determined for cell types 510 may be multiplied by the normalization coefficient to obtain the normalized cell counts.

In some embodiments, after the cell counts are normalized, the cell counts 508 and 512 are combined. In this example, because there is a leader panel, cell counts 508 may be retained when combining cell counts 508 and 512. For example, as shown, cell counts 514 include the cell counts for types A, B, and E that were included in the cell counts 508. Cell counts 514 also includes a cell count for type D, which was obtained from cell counts 512, since cell types 506 do not include cell type D.

In some embodiments, the cell composition percentages 516 are determined based on the cell combined cell counts 514. For example, each of the cell counts may be divided by the total number of cells for which cell types 506 were identified. In this example, the total number of cells includes 5 cells.

FIG. 5C depicts an illustrative example for determining cell composition percentages based on cell types determined using cytometry data obtained by performing cytometry using different panels, where neither of the panels is a leader panel, according to some embodiments of the technology described herein.

As shown, cell types 518 and cell types 522 were identified using cytometry measurements for markers included in different panels. Cell types 518 and cell types 522 each include cell types A and E. Cell types 518 include cell type B, and cell types 522 include cell type E. In some embodiments, techniques described herein may be used to determine a single cell composition percentage for each cell type.

In some embodiments, the techniques include determining, for cell types associated with each panel, respective cell composition percentages 520 and 524. For example, this may include determining a cell composition percentage (e.g., cell type A1%) for cell type A associated with panel A. The cell composition percentage may be determined based on a ratio between the number of cells of type A (e.g., 2) and the total number of cells for which cell types were identified (e.g., 5), yielding a cell composition percentage of 40%. This may be repeated for each cell type, for both panels.

In some embodiments, cell composition percentages 520 and 524 are combined to yield cell composition percentages 526, which may include a single cell composition for each cell type. For example, cell composition percentages 520 and 524 may be combined by averaging them.

FIG. 5D depicts an illustrative example for determining cell composition percentages based on beads, according to some embodiments of the technology described herein.

As shown, types 528 and types 532 were identified for particles (e.g., cells and beads) using cytometry measurements for markers included in different panels. In this example, types 528 and 532 do not include any of the same cell types. In some embodiments, techniques described herein may be used to determine a single cell composition percentage for each cell type.

In some embodiments, beads included in the subsample are used to determine normalized cell counts for cell types in the subsample. For example, the number of a particular cell type may be normalized by a number of identified beads and a known concentration of beads that were added to a biological sample. For example, presuming a concentration of 5 beads per microliter, the number of cells of cell type E (e.g., 2) associated with panel B may be normalized by multiplying the number of cells of cell type E by a ratio of the concentration of beads (e.g., 0.5 bead per microliter) and the number of beads associated with panel B (e.g., 1). In this example, the normalized cell count would be 1.

In some embodiments, the normalized cell counts are used to determine cell composition percentages 530 and 534. For example, this may include determining a ratio between the cell count and the total number of cells identified for a particular cell type associated with a panel.

For example, the cell composition percentage for cell type E may be determined based on the ratio of the normalized cell count determined for cell type E (e.g., 1) and the total number of cells identified in types 532 (e.g., 4) to yield a cell composition percentage of 25%.

In some embodiments, cell composition percentages 530 and 534 are combined to yield cell composition percentages 536.

FIG. 5E depicts an example of determining cell composition percentages based on marker composition. As shown, cytometry data was obtained using two different panels (e.g., Panel A and Panel B). Table 552 lists cells identified using cytometry data obtained for a first subsample using Panel A and table 562 lists the cells identified using cytometry data obtained for a second subsample using Panel B. A marker composition was determined for each of the cells. A cell type was determined for some of the cells listed in each table.

Cell counts 554 are determined for each of the marker compositions and cell types listed in table 552 and cell counts 564 are determined for each of the marker compositions and cell types listed in table 562. The cell counts are used to determine fractions 556 and fractions 566.

As shown FIG. 5E, the fractions may be determined separately for cells from different subsamples (e.g., the cells listed in table 552 versus the cells listed in table 554).

A fraction is determined for a particular marker composition using (a) the number of cells identified as having the particular marker composition, and (b) the number of cells identified as being of the parent cell type identified for the particular marker composition. For the purpose of this example, Cell type A is the parent cell type identified for marker compositions: M1+M2− and M1+M2− M3+. Cell type B is the parent cell type identified for the marker composition M2− M3+M4− M5+. As shown in table 5E, a single fraction is determined for marker composition M1+M2− M3+ based on the cell count for marker composition M1+M2− M3+ and the cell count for parent cell type A, each of which are listed in table 554. By contrast, because marker composition M1+M2− was identified for cells in both subsamples, two fractions were determined for M1+M2−. The first fraction, listed in table 556, is determined using the counts listed in table 554. The second fraction, listed in table 566, is determined using the counts listed in table 564.

As shown in FIG. 5E, the marker composition M1+M2− M3+ has a parent marker composition M1+M2− because M1+M2− M3+ includes all the markers (e.g., M1+M2−) of its parent marker composition. Furthermore, the parent and child marker compositions were both identified for cells in the same subsample. Therefore, the fraction determined for M1+M2− M3+ may be normalized by the fraction determined for M1+M2− to obtain a normalized fraction.

Fractions 556, normalized fractions 558, and fractions 566 are used to obtain average fractions for the marker compositions, as listed in table 570. Because M1+M2− is the only marker composition to be identified cells in both subsamples (resulting in two fractions), only the fractions determined for M1+M2− are averaged (e.g., the normalized fraction listed in table 558 and the fraction listed in table 566).

The average fractions are used to determine the composition percentage of cells having the particular marker composition in a subject from which the subsamples were obtained. In particular, as shown in table 570, the cell composition percentage for a particular marker composition is obtained by multiplying the average fraction determined for the marker composition by the cell composition percentage determined for the parent cell type identified for the marker composition. Similarly, a cell concentration can be obtained for the marker composition.

TABLE 13
Example hierarchy of cell types.

| Cell type | Cell subtype |
|---|---|
| Generic | Leukocytes |
| Leukocytes | Granulocytes |
| Granulocytes | Eosinophils |
| Granulocytes | Neutrophils |
| Granulocytes | Basophils |
| Leukocytes | Monocytes |
| Monocytes | Classical monocytes |
| Classical monocytes | Classical monocytes FceRI+ |
| Classical monocytes | Classical monocytes FceRI− |
| Monocytes | Non-classical monocytes |
| Leukocytes | Dendritic cells |
| Dendritic cells | CDC |
| CDC | cDC1 |
| CDC | cDC2 |
| Dendritic cells | Plasmacytoid dendritic cells |
| Leukocytes | Lymphocytes |
| Lymphocytes | B cells |
| B cells | Naïve B cells |
| B cells | Memory B cells |
| Memory B cells | Non-switched Memory IgM B cells |
| Memory B cells | Class-switched Memory |
| Class-switched Memory | Switched Memory IgG+ |
| Class-switched Memory | Switched Memory IgA+ |
| B cells | Secreting abs B cells |
| Secreting abs B cells | Plasmablasts |
| Plasmablasts | Plasmablasts IgA+ |
| Plasmablasts | Plasmablasts IgG+ |
| Secreting abs B cells | Plasma cells |
| Plasma cells | Plasma cells IgA+ |
| Plasma cells | Plasma cells IgG+ |
| Lymphocytes | NK cells |
| NK cells | Immature NK cells |
| NK cells | Mature NK cells |
| Mature NK cells | Mature CD158+ |
| Mature CD158+ | Mature NK CD158+ CD57+ |
| Mature NK cells | Mature CD158− |
| Lymphocytes | T cells |
| T cells | NKT cells |
| T cells | HLA-DR T cells |
| T cells | gdT cells |
| T cells | iNKT |
| T cells | MAIT cells |
| MAIT cells | MAIT CD8+ |
| MAIT cells | MAIT CD8− |
| T cells | CD4 T cells |
| CD4 T cells | CD4 Tregs |
| CD4 Tregs | CD4 Naive Tregs |
| CD4 Tregs | CD4 Memory Tregs |
| CD4 T cells | CD4 T helpers |
| CD4 T helpers | CD4 Naïve T cells |
| CD4 T helpers | CD4 Memory T helpers |
| CD4 Memory T helpers | CD4 Central Memory |
| CD4 Central Memory | CD4 Central Memory CCR4− CCR6− CXCR3+ CXCR5− |
| CD4 Central Memory | CD4 Central Memory CCR4+ CCR6+ CXCR3− CXCR5− |
| CD4 Central Memory | CD4 Central Memory CCR4+ CCR6− CXCR3− CXCR5− |
| CD4 Memory T helpers | CD4 Transitional Memory |
| CD4 Transitional Memory | CD4 Transitional Memory CCR4− CCR6− CXCR3+ |
| CD4 Transitional Memory | CD4 Transitional Memory CCR4+ CCR6+ CXCR3− |
| CD4 Transitional Memory | CD4 Transitional Memory CCR4+ CCR6− CXCR3− |
| CD4 Memory T helpers | CD4 Effector Memory |
| CD4 Effector Memory | CD4 Effector Memory CCR4+ CCR6+ CXCR3− CXCR5− |
| CD4 Effector Memory | CD4 Effector Memory CCR4+ CCR6− CXCR3− CXCR5− |
| CD4 Effector Memory | CD4 Effector Memory CCR4− CCR6− CXCR3+ CXCR5− |
| CD4 Memory T helpers | CD4 TEMRA |
| CD4 Memory Tregs | CD4 Memory Tregs CD39+ |
| CD4 Memory Tregs | CD4 Memory Tregs CD39− |
| CD4 Memory Tregs CD39+ | CD4 Memory Tregs CD39+ ICOS+ |
| CD4 Memory T helpers | CD4 Memory CD39+ |
| CD4 Memory T helpers | CD4 Memory CD39− |
| T cells | CD8 T cells |
| CD8 T cells | CD8 Naive T cells |
| CD8 Naive T cells | CD8 Stem Cell Memory CD57− CD95+ |
| CD8 Naive T cells | CD8 True Naive T cells |
| CD8 T cells | CD8 Memory T cells |
| CD8 Memory T cells | CD8 Central Memory |
| CD8 Memory T cells | CD8 Transitional Memory |
| CD8 Memory T cells | CD8 Effector Memory |
| CD8 Memory T cells | CD8 TEMRA |
| CD8 Central Memory | CD8 Central Memory PD-1+ |
| CD8 Central Memory | CD8 Central Memory PD-1− |
| CD8 Central Memory PD-1+ | CD8 Central Memory PD-1+ CD39+ |
| CD8 Effector Memory | CD8 Effector Memory PD-1+ |
| CD8 Effector Memory | CD8 Effector Memory PD-1− |
| CD8 Effector Memory PD-1+ | CD8 Effector Memory PD-1+ CD39+ |
| CD8 Transitional Memory | CD8 Transitional Memory PD-1+ |
| CD8 Transitional Memory | CD8 Transitional Memory PD-1− |
| CD8 Transitional Memory PD-1+ | CD8 Transitional Memory PD-1+ CD39+ |
| CD8 TEMRA | CD8 TEMRA PD-1+ |
| CD8 TEMRA | CD8 TEMRA PD-1− |
| CD8 TEMRA PD-1+ | CD8 TEMRA PD-1+ CD39+ |
| CD8 Central Memory | CD8 Central Memory CD57+ |
| CD8 Central Memory | CD8 Central Memory CD57− |
| CD8 Effector Memory | CD8 Effector Memory CD57+ CD95+ |
| CD8 Effector Memory | CD8 Effector Memory CD57− CD95+ |
| CD8 Effector Memory CD57+ CD95+ | CD8 Effector Memory CD57+ CD95+ CX3CR1+ |
| CD8 Effector Memory CD57− CD95+ | CD8 Effector Memory CD57− CD95+ CX3CR1− |
| CD8 Transitional Memory | CD8 Transitional Memory CD57+ |
| CD8 Transitional Memory | CD8 Transitional Memory CD57− |
| CD8 TEMRA | CD8 TEMRA CD57+ |
| CD8 TEMRA | CD8 TEMRA CD57− |

### Example Reports

FIGS. 6A-6E are screenshots of an example report indicating information for multiple cell populations of particular types in a biological sample. The biological sample may have been obtained from a subject having, suspected of having or at risk of having cancer. As shown in FIG. 6A, the report may indicate a type for the biological sample, a diagnosis for the subject, a therapy being used to treat the subject, a stage of a disease for which the subject is diagnosed, or any other suitable information.

FIGS. 6A and 6B indicate, for each of multiple cell populations, results associated with analyzing the cell populations according to embodiments of the technology described herein. For example, the report indicates, for each cell population, a cell concentration and cell composition percentage of the cell population in the biological sample. The report also indicates, for each cell population, ranges of cell concentrations and cell composition percentages associated with a cohort of healthy patients. An indication of high or low may highlight cell concentrations or composition percentages that are high or low relative to the ranges associated with the cohorts of healthy patients.

FIG. 6C shows a visualization that represents, for each cell population, how much the cell composition percentage determined for the biological sample differs from the range of cell composition percentages associated with the cohort of health of patients.

FIG. 6D indicates cell concentrations measured for different cell populations using a hematology analyzer. The cell concentrations are compared to ranges of cell concentrations associated with cohorts of healthy patients.

FIG. 6E shows a visualization that represents a patient's predicted prognosis and/or response to a particular therapy based on the determining cell compositions of a sample. For example, the visualization indicates whether the number of cells of a particular population (e.g., CD8+PD1+ cells, CD4+ cells, and NK cells) is high or low. The visualization also indicates whether a ratio of different cell populations (e.g., PD1+CD8+ T cells to PD1+CD4+ T cells ratio, CD4+ T cells to lymphocytes ratio, and CD4+ T cells to CD8+ T cells ratio) is high or low. The visualization also indicates, based on the size, whether the patient will have a positive or negative response to a particular treatment, or whether the patient has a superior or inferior prognosis.

It should be appreciated that the visualizations shown in FIGS. 6A-6E are exemplary, and that alternative visualizations are possible.

### Machine Learning Training

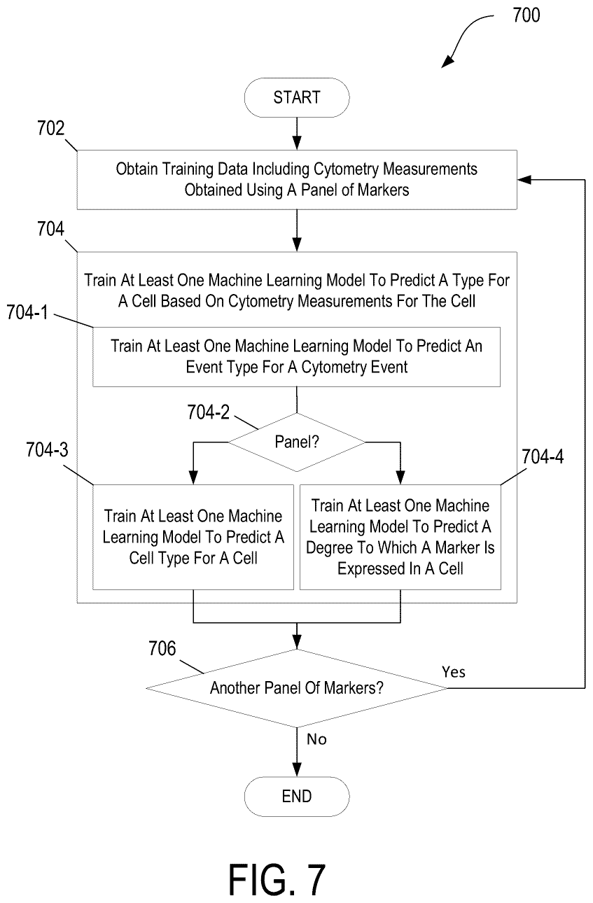

FIG. 7 is a flowchart of an illustrative process 700 for training one or more machine learning models, according to some embodiments of the technology described herein. Process 700 may be performed by a laptop computer, a desktop computer, one or more servers, in a cloud computing environment, computing device 108 as described herein with respect to FIG. 1D, computing device 1200 as described herein with respect to FIG. 12, or any other suitable computing device(s), as aspects of the technology described herein are not limited in this respect.

At act 702, training data is obtained. In some embodiments, the training data may be obtained in a clinical or research setting, from a data store storing such information, and/or from any suitable source, as aspects of the technology described herein are not limited in this respect.

In some embodiments, the training data includes cytometry data including cytometry measurements obtained during respective cytometry events. For example, measurements obtained during a first event may be included in the cytometry data, where the first event corresponds to a cell being measured by the cytometry platform. Examples of cytometry data are described herein including at least in the “Flow Cytometry” and “Mass Cytometry” sections.

In some embodiments, the cytometry measurements are obtained using a panel of markers. In some embodiments, the cytometry measurements include measurements for markers included in the panel of markers. The panel of markers may include any suitable number of markers such as, for example, at least 2 markers, at least 5 markers, at least 7 markers, at least 10 markers, at least 12 markers, at least 15 markers, at least 30 markers, between 2 and 40 markers, between 5 and 15 markers, or any other suitable number of markers.

In some embodiments, the cytometry data is processed using any suitable processing techniques such as those described herein including at least with respect to act 204 of process 200 in FIG. 2A and those described in the Training Data section.

In some embodiments, the cytometry data is labeled. For example, cytometry measurements obtained during a cytometry event may be labeled to indicate an event type for the event. The event type may indicate whether the particular event corresponds to a cell being measured by the cytometry platform, or a bead being measured by the cytometry platform. In some embodiments, when the event type indicates that an event corresponds to a cell being measured by the cytometry platform, the particular event may additionally be labelled to indicate a cell type for the event and/or labelled to indicate a degree of expression of one or more markers corresponding to the cell type (e.g., the label may indicate whether a particular marker is positive or negative).

In some embodiments, the cytometry data is divided into N batches. In some embodiments, N is any suitable number of one or more batches. As a nonlimiting example, N may be based on a number of flow cytometry standard (FCS) files that include the training data obtained at act 702. For example, N may be the total number of FCS files obtained at act 702 divided by 10. The resulting total may be rounded down.

At act 704, at least one machine learning model is trained to predict a type for a cell based on cytometry measurements for the cell. As described herein, the at least one machine learning model may include one or more machine learning models trained to predict an event type for an event, one or more machine learning models trained to predict a cell type for an event, and/or one or more machine learning models trained to predict a degree of expression of each of one or more markers in a cell.

At act 704-1, at least one machine learning model is trained to predict an event type for a cytometry event. In some embodiments, this involves training N machine learning models to predict an event type for the cytometry event using the N batches of training data. For example, this may include training a multi-class classifier to predict the event type (e.g., cell, bead, doublet, debris, undefined, etc.). Additionally, or alternatively, this may include training multiple binary classifiers to predict the likelihood that the object measured during the event is of particular type. For example, this may include training a first binary classifier to predict whether the object measured during the event is a cell, a second binary classifier to predict the whether the object measured during the event is a bead, a third binary classifier to predict whether the object measured during the event is an undefined object, and so on.

In some embodiments, for each of the N batches of training data, cytometry measurements are concatenated into a matrix X, and corresponding event labels are concatenated into a vector Y to obtain a training input (X,Y). Accordingly, in some embodiments, N machine learning models are trained, using respective training input (X_{N}, Y_{N}) to predict an event type for cytometry measurements obtained during a cytometry event using the panel of markers for which the training data was obtained.

In some embodiments, the machine learning model(s) may be trained using any suitable training technique(s), including supervised techniques, semi-supervised techniques, unsupervised techniques, or any suitable combination thereof as aspects of the technology described herein are not limited in this respect. As one example, in the supervised training context, the cytometry measurements may be provided as input to the machine learning model, which may output a predicted event type. Differences between the predicted event type and the known event type (e.g., the event type indicated by the corresponding event type label) may be used to determine and update the parameter values of the machine learning model.

Decision 704-2 indicates that, in some embodiments, the type of machine learning trained during process 700 may depend on the panel of markers used to obtain the training data. For example, in some embodiments, training data obtained using one panel of markers may be used to train at least one machine learning model to predict a cell type for a cell, while training data obtained using a different panel of markers may be used to train at least one machine learning model to predict a degree to which a marker is expressed in a cell. Example panels and corresponding machine learning types are listed in Tables 1A-10C.

In some embodiments, at act 704-3, at least one machine learning model is trained to predict a cell type for a cell. In some embodiments, this includes, for each of the N batches of training data, training a machine learning model (e.g., a multiclass classifier or one or more binary classifiers) to predict a cell type for a cell from among multiple cell types.

In some embodiments, for each of the N batches of training data, cytometry measurements of cells are concatenated into a matrix X, and corresponding cell type labels are concatenated into a vector Y to obtain a training input (X,Y). Accordingly, in some embodiments, N machine learning models are trained, using respective training input (X_{N}, Y_{N}), to predict a cell type for cytometry measurements obtained of a cell using the panel of markers for which the training data was obtained. As one example, a multiclass classifier may be trained to predict a type for a cell from among the cell types listed in Table 10A using cytometry measurements obtained for the markers in Table 10A and corresponding labels indicating types for cells that generated the cytometry measurements (e.g., as a result of processing the cells using the cytometry platform).

In some embodiments, the machine learning model(s) may be trained using any suitable training technique(s), including supervised techniques, semi-supervised techniques, unsupervised techniques, or any suitable combination thereof as aspects of the technology described herein are not limited in this respect. As one example, in the supervised training context, the cytometry measurements may be provided as input to the machine learning model, which may output a predicted cell type. Differences between the predicted cell type and the known cell type (e.g., the event type indicated by the corresponding cell type label) may be used to determine and update the parameter values of the machine learning model.

In some embodiments, at act 704-4, at least one machine learning model is trained to predict the degree to which a marker is expressed in a cell. In some embodiments, this includes, for each of the N batches of training data, training each machine learning model in a set of one or more machine learning models (e.g., one or more binary classifiers) to predict the degree to which a respective marker is expressed in a cell. Accordingly, in some embodiments, N sets of one or more machine learning models are trained at act 704-4 to predict degrees of marker expression using cytometry measurements obtained of a cell using the panel of markers for which the training data was obtained.

In some embodiments, a subset of the training data obtained at act 702 is used to train a machine learning model to predict the degree to which a particular marker is expressed in a cell. For example, the subset of training data may include cytometry measurements of cells associated with expression of that marker. Tables 1B, 2C, 7B, 8C, and 10C list examples of cell types and corresponding markers. The cytometry measurements included in the subset of training data may be concatenated into a matrix X, and the corresponding degree of expression labels may be concatenated into a vector Y to obtain a training input (X,Y). As one example, a binary classifier may be trained to predict a degree to which a cell expresses CD8 using cytometry measurements obtained for the markers listed in Table 1A and a corresponding label indicating the degree to which the corresponding cell used to generate the cytometry measurements expressed CD8.

In some embodiments, the machine learning model(s) may be trained using any suitable training technique(s), including supervised techniques, semi-supervised techniques, unsupervised techniques, or any suitable combination thereof as aspects of the technology described herein are not limited in this respect. As one example, in the supervised training context, the cytometry measurements may be provided as input to the machine learning model, which may output a predicted degree of marker expression. Differences between the predicted event type and the known event type (e.g., the event type indicated by the corresponding degree of marker expression label) may be used to determine and update the parameter values of the machine learning model.

At act 706, process 700 includes determining whether there is another panel of markers for which one or more machine learning models are to be trained. For example, a different panel of markers may be used to obtain cytometry measurements that can be used to distinguish between additional cell types. Such cell types may not be distinguishable using the machine learning models that have already been trained using process 700. If there is another panel of markers, then process 700 may return to act 702, otherwise process 700 ends.

As described herein, process 700 may be used to train N machine learning models to predict an event type for cytometry measurements obtained during a cytometry event using the panel of markers for which the training data was obtained, N machine learning models are trained to predict a cell type for cytometry measurements obtained of a cell using the panel of markers for which the training data was obtained, and/or N sets of one or more machine learning models are trained to predict degrees of marker expression for cytometry measurements obtained of a cell using the panel for which the training data was trained.

In some embodiments, the N sets of models may be used for bagging to reduce classification error. For example, the N sets of models may be used to generate N partially hierarchical models.

In some embodiments, a partially hierarchical model includes a machine learning model trained to predict an event type and a machine learning model trained to predict a cell type. In some embodiments, the output of such a model indicates a cell type for a cells. When bagging is used, in some embodiments, N outputs are obtained, each of which includes a predicted cell type for a cell. In some embodiments, the mode of the N output is identified as the cell type for the cell.

In some embodiments, a partially hierarchical model includes a machine learning model trained to predict an event type and a set of machine learning models trained to predict degrees of marker expression. In some embodiments, the output of such a model indicates, for each of one or more markers, the degree of the expression of the particular marker in a cell (e.g., whether the marker is positive or negative). When bagging is used, in some embodiments, N outputs are obtained, each of which includes a set of one or more degrees of marker expression. In some embodiments, the mode of the N output is identified as the degrees of marker expression of the cell.

### Training Data

As described herein, training the plurality of machine learning models includes obtaining training data including cytometry data for cells and the corresponding cell types. In some embodiments, obtaining the training data includes obtaining cytometry data for one or more biological samples and manually processing the cytometry data to determine types for cells in the biological sample.

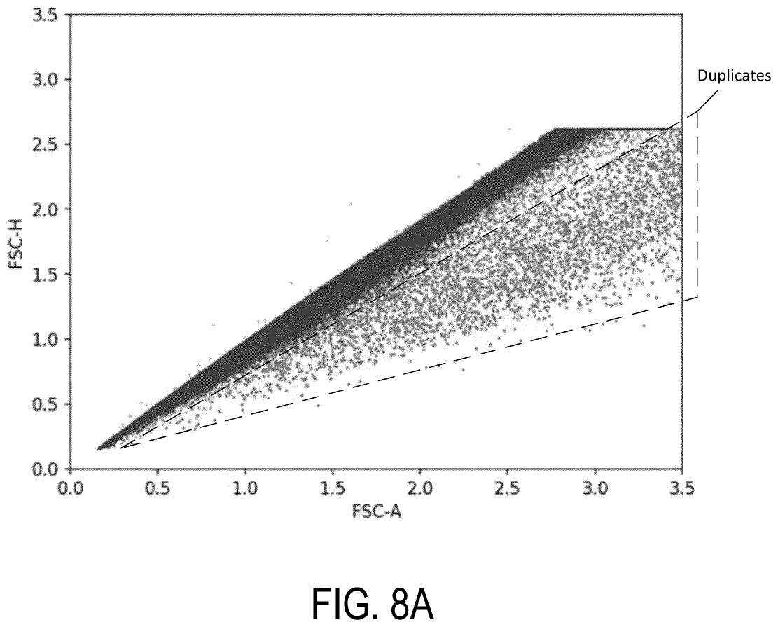

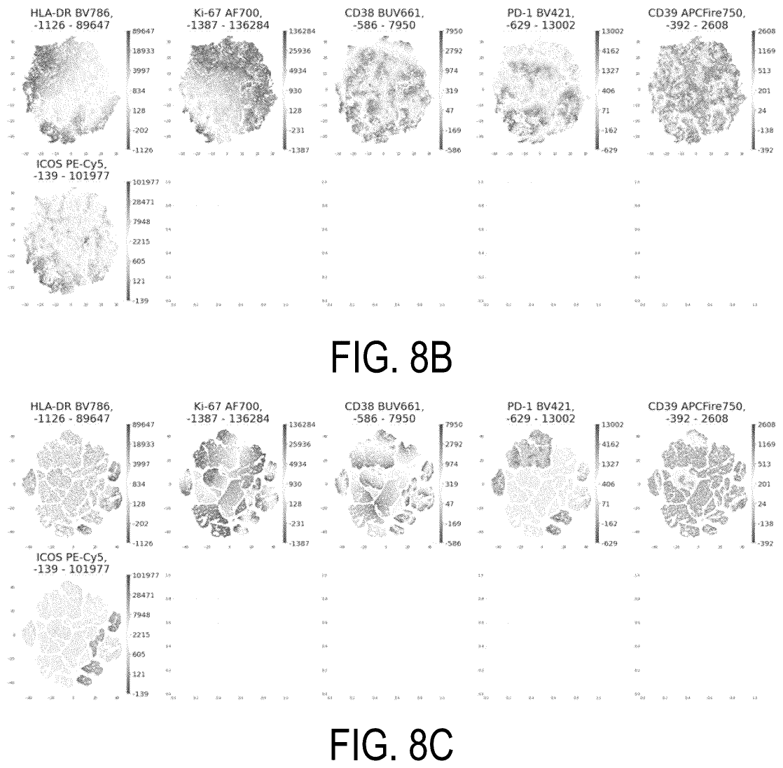

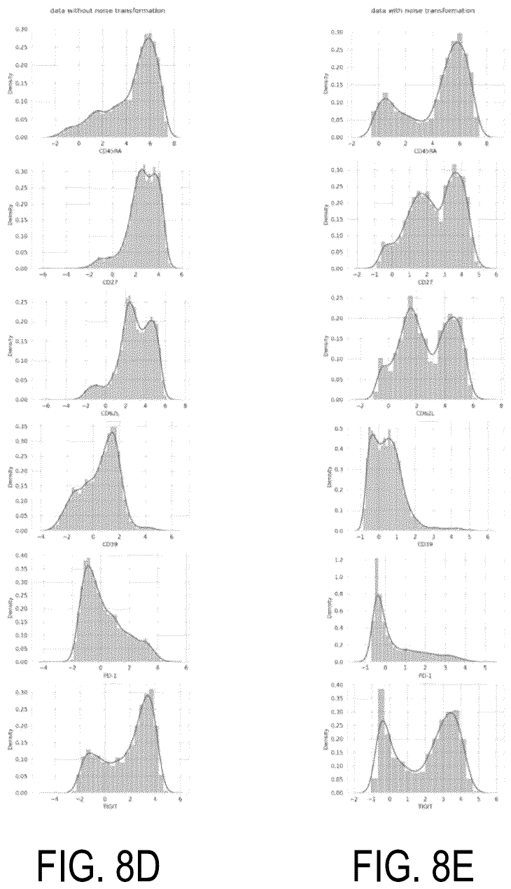

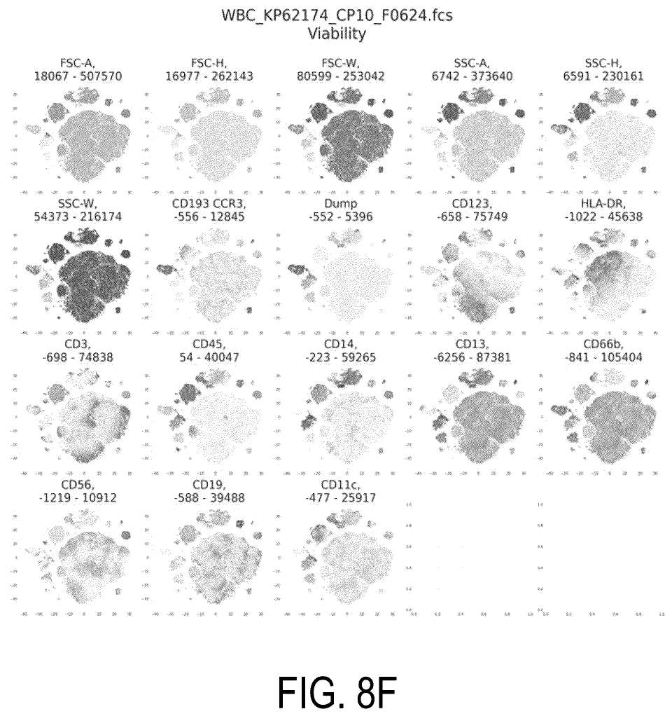

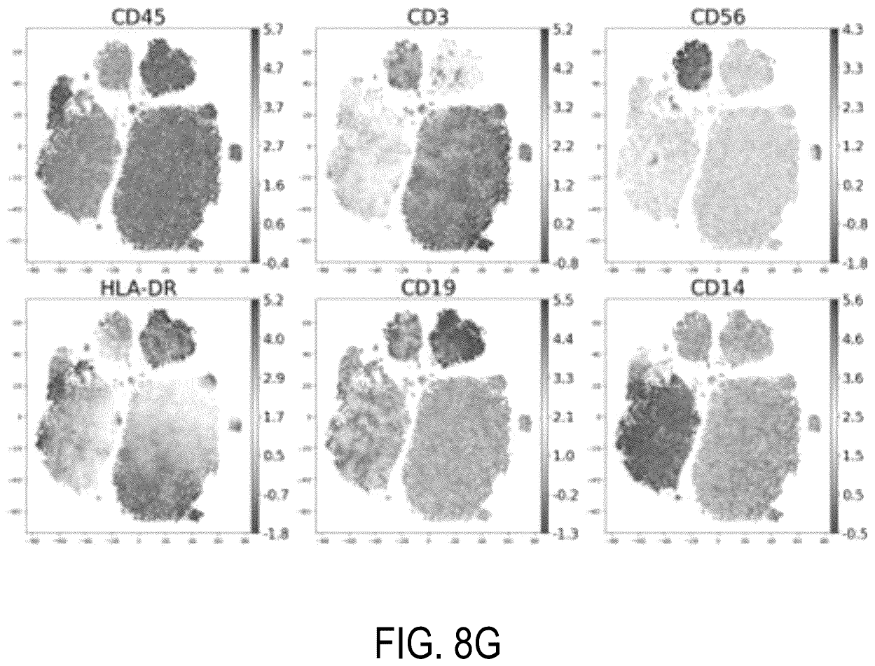

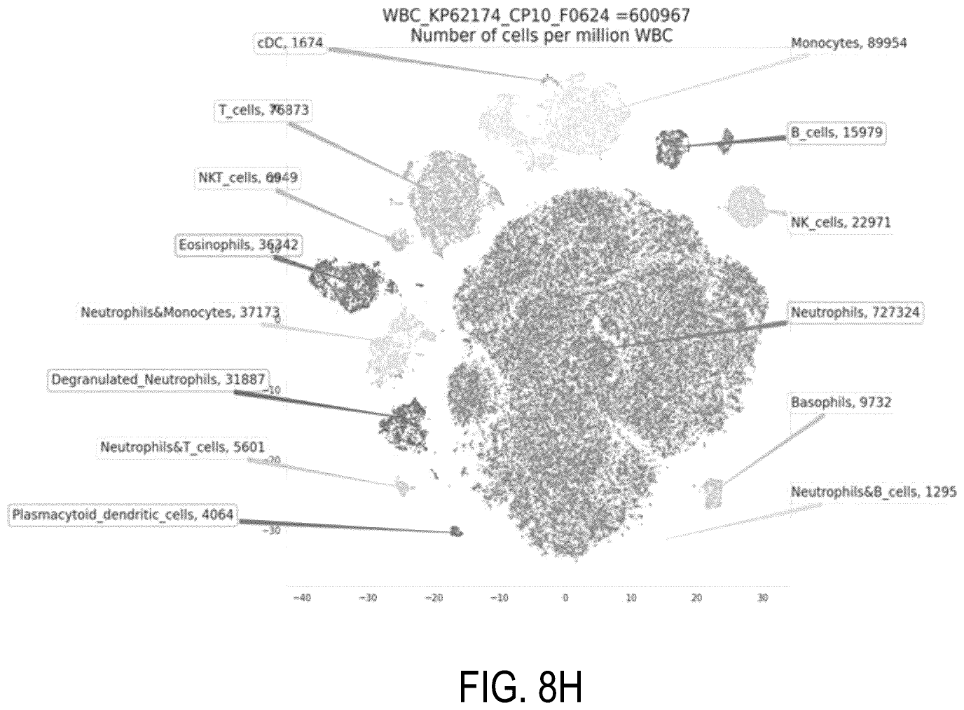

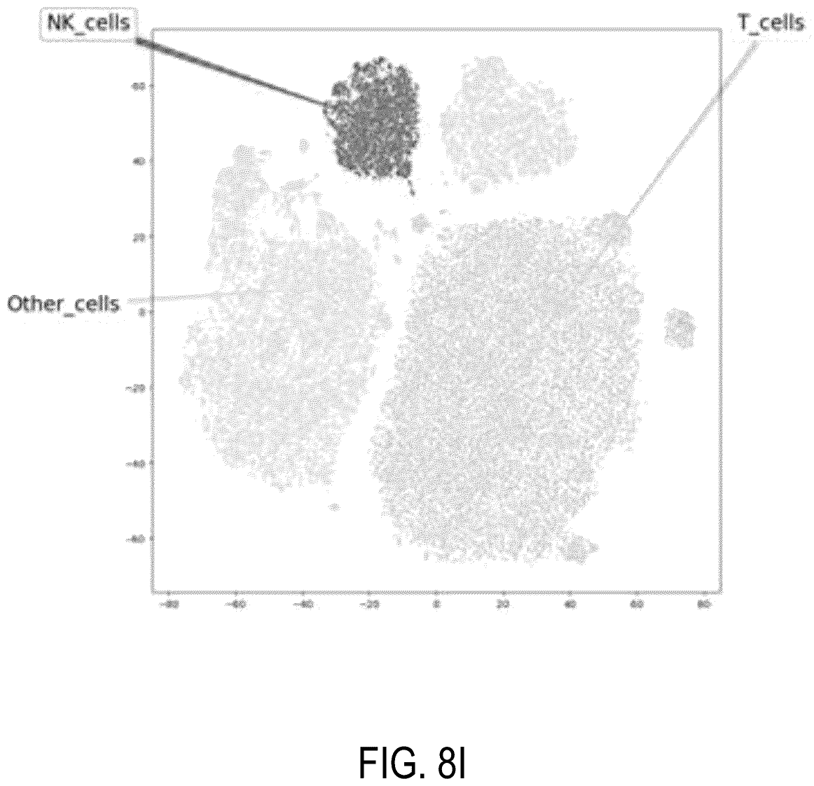

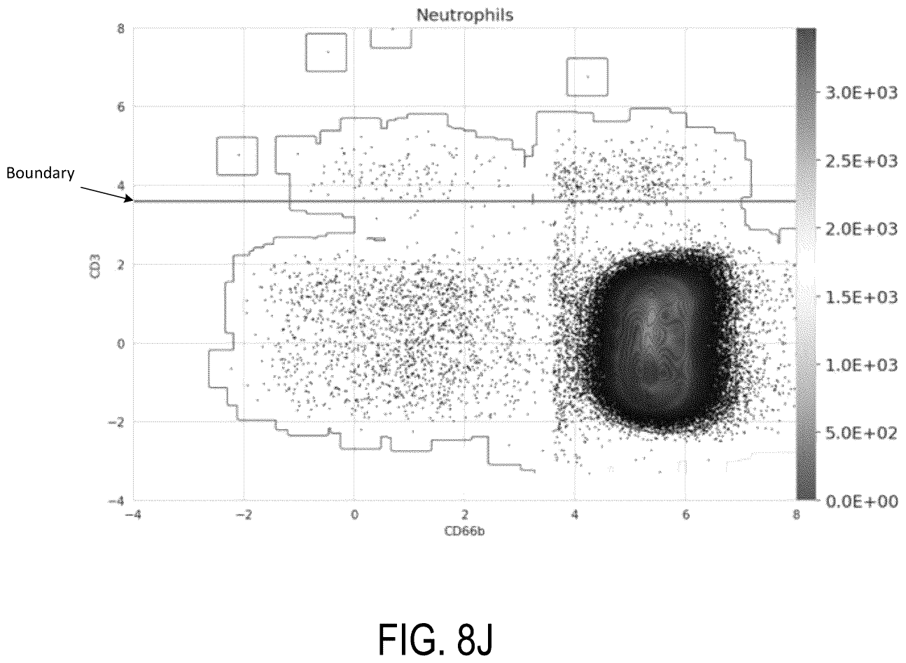

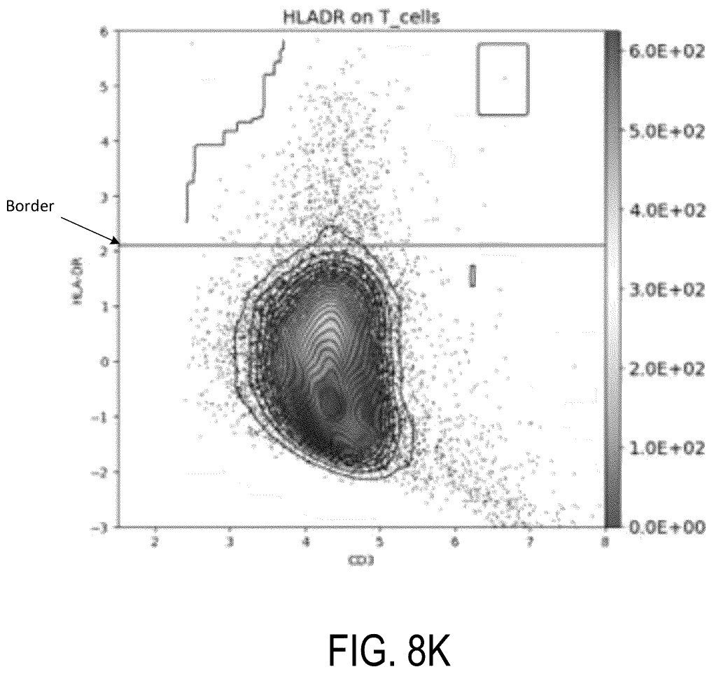

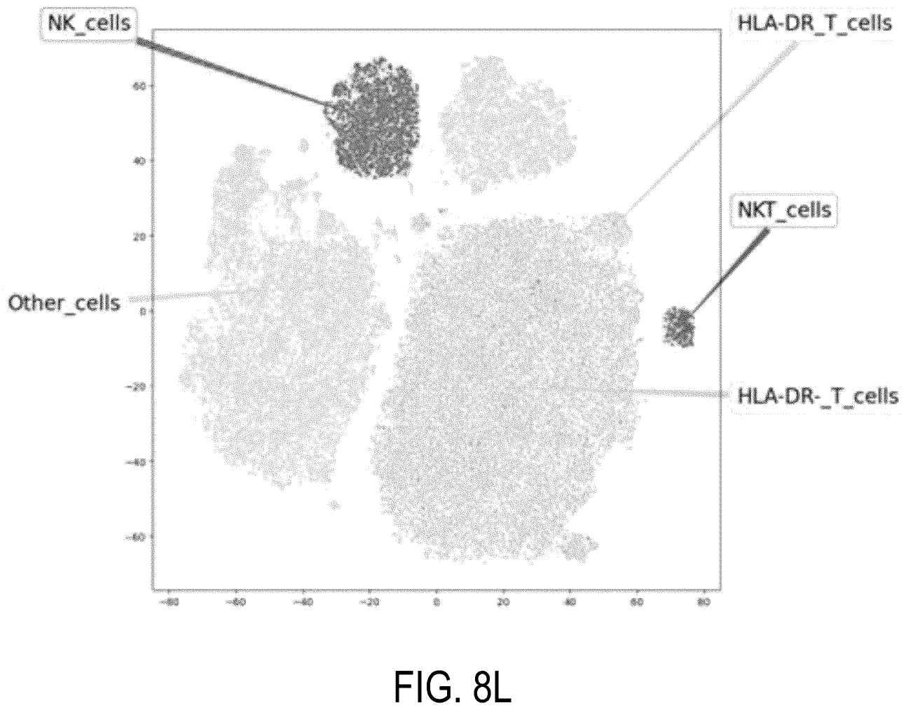

In some embodiments, processing the cytometry data may include gating the cytometry data. For example, this may include manually gating the cytometry data to separate discrete cell populations based on shared marker expression. In some embodiments, gating may be performed using any suitable gating techniques, such as by using FlowJo™ (FlowJo™ Software. Ashland, OR: Beckton, Dickinson and Company; 2021). In some embodiments, the gating analysis is implemented in a suitable programming language (e.g., Python). In some embodiments, gating includes generating two-dimensional plots of marker intensities. For example, FIG. 8A shows a plot of forward scatter height (FSC-H) against forward scatter area (FSC-A). The plot may be used to identify duplicate events, which may be discarded and excluded from further analysis.

In some embodiments, gating may result in a file (e.g., a Workspace (WSP) file) that includes any suitable information about the gating, such as information about the coordinates of the gates, axes transformation, statistics, and layouts.

In some embodiments, processing the cytometry data may additionally or alternatively include clustering the cytometry data for a sample. This may include calculating two-dimensional t-SNE plots for a sample and calculating FlowSOM for the sample. FlowSOM is described by Van Gassen et al. (“FlowSOM: Using self-organizing maps for visualization and interpretation of cytometry data,” in Journal of Quantitative Cell Science, vol. 87, no. 7, pp. 636-645, 2015), which is incorporated by reference herein in its entirety.

Prior to clustering, in some embodiments, processing the cytometry data may include a noise transformation of the cytometry data. This may include transforming the intensity of the markers to reduce the influence of noise on the clustering results. In some embodiments, transforming the intensity of a marker includes reducing the intensity of the marker lower than a specified border. Such a border may be identified based a result of gating or using Fluorescence Minus One controls. In some embodiments, a border is defined as a border between a positive signal from a marker and the intensity of noise in the channel of the marker. Equations 9 and 10 describe the intensity of a marker after the noise transformation (I_{after transform}):

$${I}_{{after transform}^{}} = {I}_{initial} , if {I}_{initial} \geq border \qquad ( Equation 9 )$$

$${I}_{{after transform}^{}} = \frac{{I}_{initial}}{k} , if {I}_{initial} < border \qquad ( Equation 10 )$$

where Iᵢₙᵢₜᵢₐₗ is the initial intensity of the marker from the cytometry data, border is the border of reduction for the intensity of the marker, and k is the coefficient of reduction. In some embodiments, the coefficient of reductions is a constant, user-defined value. In some embodiments, the coefficient of reduction linearly increases from 1 at the border value to a user-defined maximum value at the minimum intensity of the marker.

FIGS. 8B-8C show the difference between clustered cytometry data before the noise transformation and after the noise transformation. As shown in FIG. 8C, the clusters are more distinct from one another after the noise transformation.

FIGS. 8D-8E show the difference between the distribution of marker intensities before the noise transformation and after the noise transformation. As shown in FIG. 8E, the distributions of marker after the noise transformation more closely resemble bimodal distributions.

Regardless of whether the noise transformation techniques are used, after clustering, the techniques may include plotting t-SNE multiplot with the intensity of markers and scatter light.

Example plots are shown in FIG. 8F and FIG. 8G. In some embodiments, each point may correspond with the value of a cell, particle, or debris for which cytometry data was obtained. In some embodiments, the plots may be used to identify different clusters, which may correspond to populations of cells, particles, or debris.

In some embodiments, a user may manually label the clusters with a corresponding cell type. For example, as shown in FIG. 8H and FIG. 8I, different clusters are labeled with different cell types. Points within each labeled cluster may correspond to a particular cell, particle, or debris in the cytometry data.

In some embodiments, an automatic labeling algorithm is used to label the clusters with corresponding cell types. A label may be selected based on the positive or negative signals from the specified markers. A positive signal from a marker is when the intensity of the marker is greater than, or equal to, the threshold. A negative signal from the marker is when the intensity of the marker is less than the threshold. For example, the automatic labeling algorithm may determine the average marker intensity within a cluster and compare it to a threshold. The threshold may be chosen by an operator based on a shift in the marker intensity distribution, determined automatically in a Fluorescence Minus One (FMO) experiment, or determined using any other suitable techniques, as aspects of the technology described herein are not limited in this respect.

In some embodiments, the techniques may further include discarding some of the identified clusters. For example, clusters corresponding to debris and/or particles may be discarded. In some embodiments, the steps for calculating and plotting the t-SNE plots and for labelling the clusters may be repeated without the discarded clusters.

In some embodiments, clustering quality may be enhanced by examining the intensity levels of various markers for events within a specified population. In some embodiments, events with inconsistent intensity (e.g., relative to the intensity of markers for other events in the population) are removed from the cluster. This may help to ensure high quality of the data. For example, as shown in FIG. 8J, events having marker (e.g., CD3) intensities about the boundary line may be discarded.

In some embodiments, two-dimensional plots of marker intensities with point density coloring and/or Kernel Density Estimation lines are estimated. An example of such a plot is shown in FIG. 8K. In some embodiments, the density may be used to further distinguish between cell populations. For example, lower density points may correspond to events that should be excluded from a particular population and/or are part of a different population. For example, in FIG. 8K, the border is used to distinguish HLA-DR+ T cells from HLA-DR− T cells.

FIG. 8L shows an example of the refined cell populations (e.g., relative to FIG. 8I) resulting from removing events with inconsistent intensities and distinguishing between cell populations based on density. As described herein, the cell populations may be labeled manually or automatically.

While various techniques for processing cytometry data have been described, it should be appreciated that any suitable techniques may be used to process such data, as aspects of the technology described herein are not limited in this respect.

### Compensation

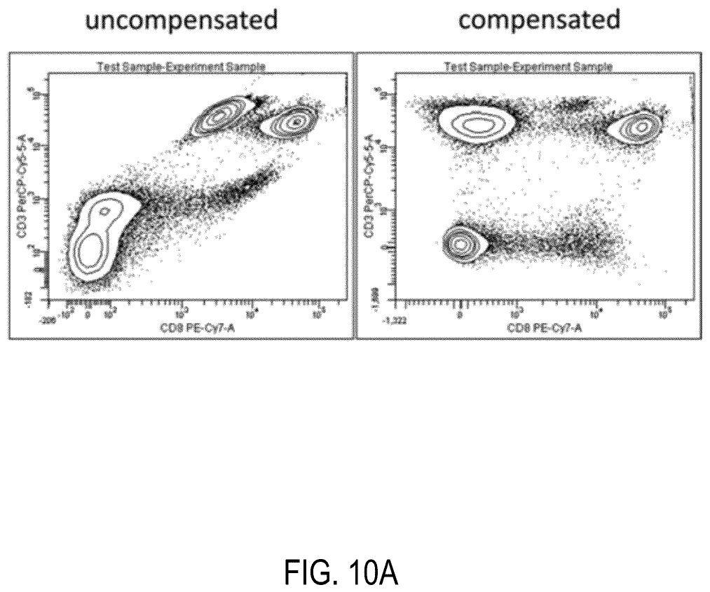

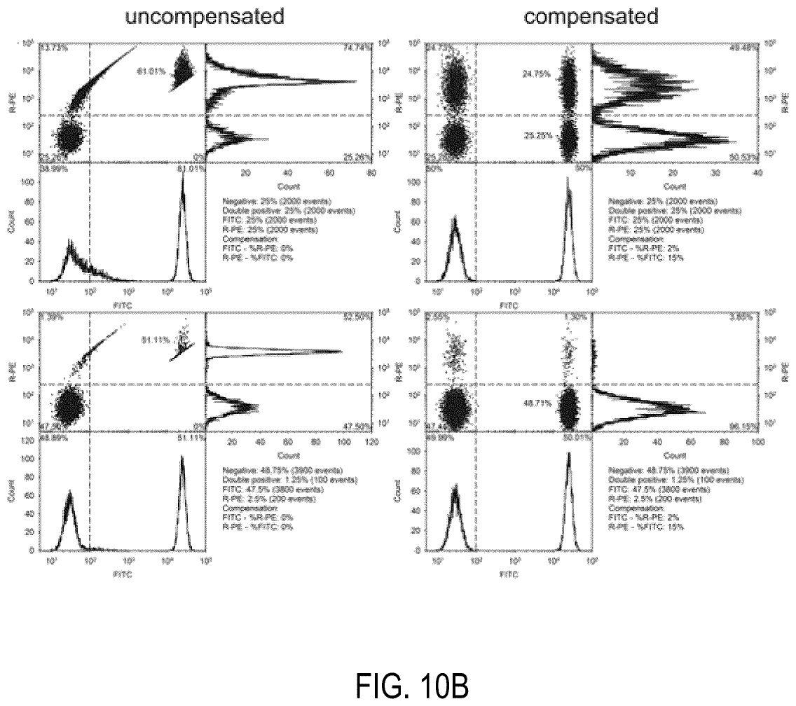

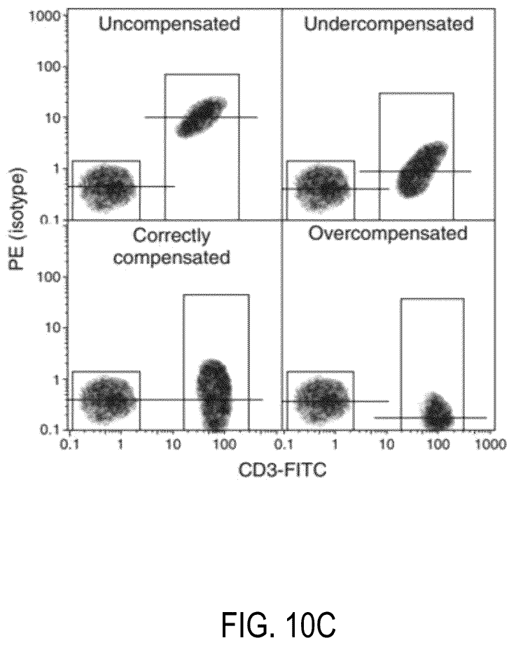

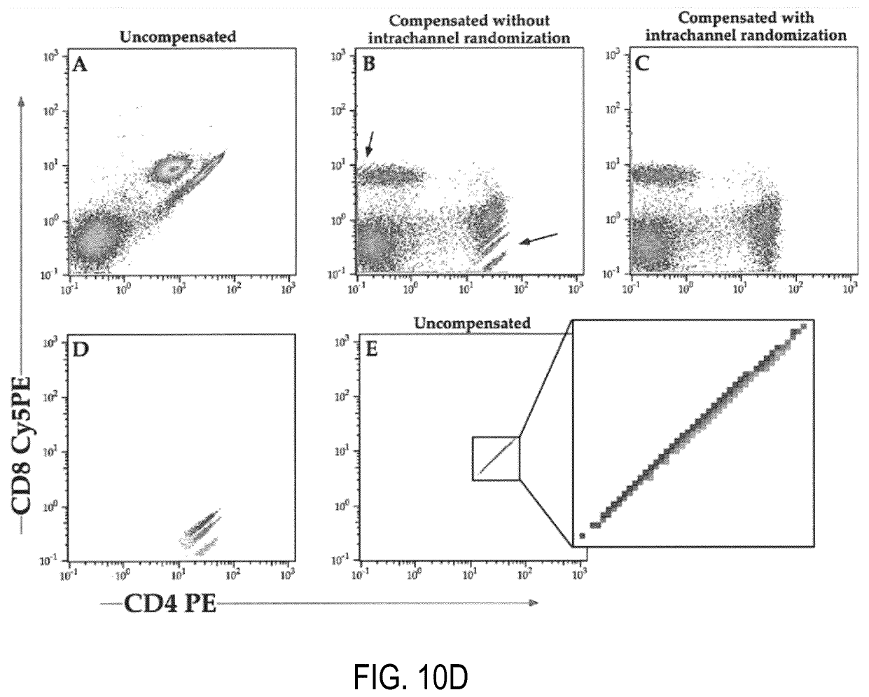

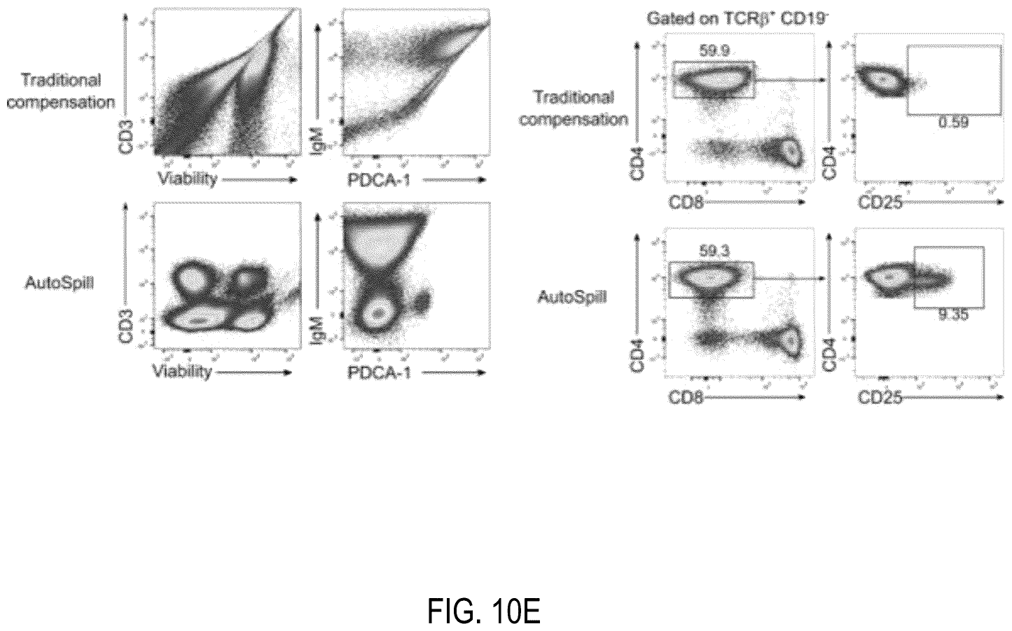

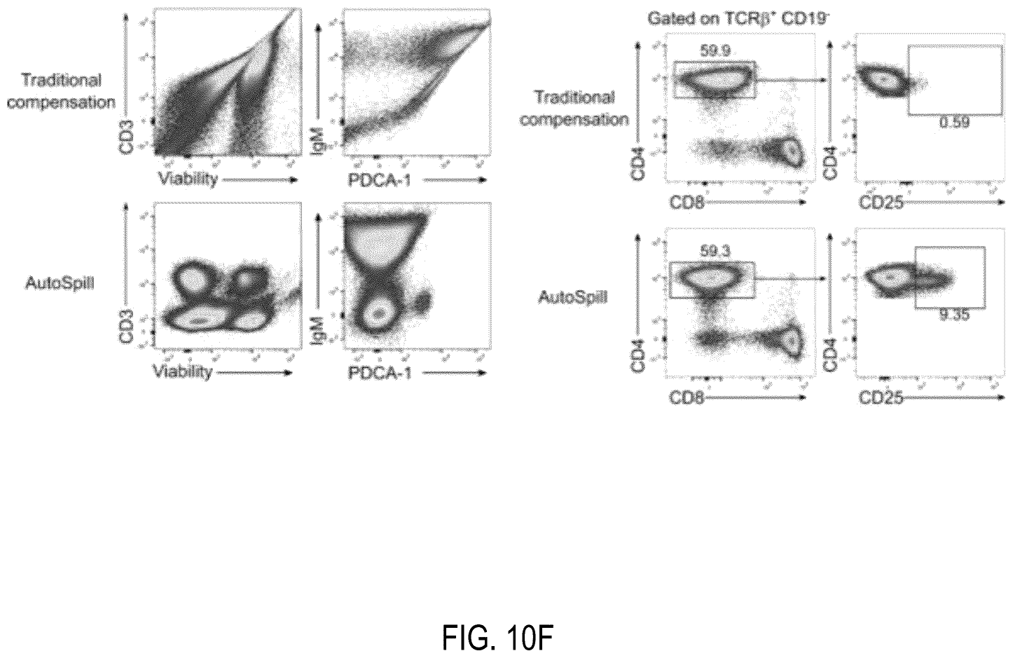

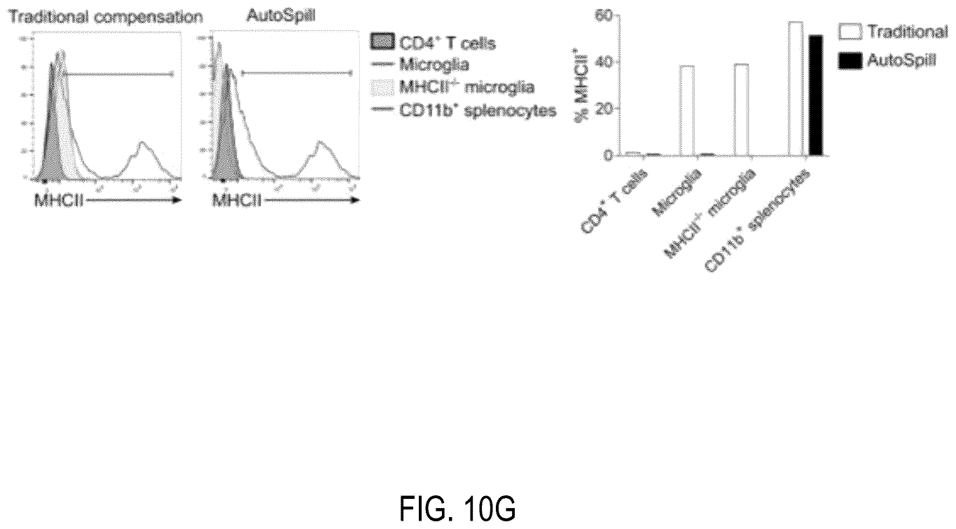

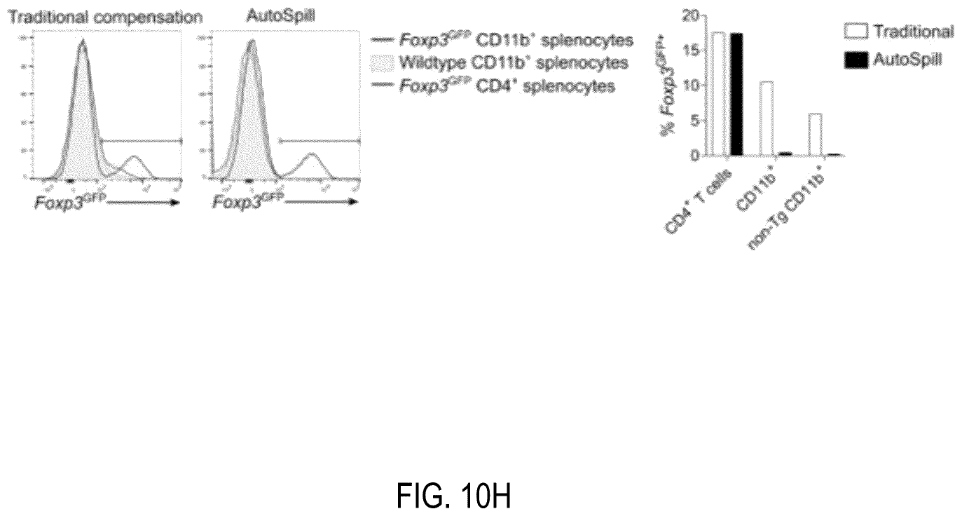

As described herein, flow cytometry employs fluorochromes, which frequently have overlapping excitation and emission spectra. Consequently, signal detection occurs not only in the primary channel, but also in neighboring channels. This is often referred to as “spillover.” Spillover is correlated with the original signal in an approximately linear manner and can be corrected using a technique called “compensation.” Compensation may be performed to remove the signal of a fluorochrome from detectors other than the one devoted to measuring that fluorochrome. Examples of compensated versus uncompensated cytometry data are shown in FIG. 10A and FIG. 10B. As shown in FIG. 10B, lack of compensation leads to the incorrect fusion of double-positive and FITC-positive cell populations. By contrast, compensation leads to the separation of the double-positive and FITC-positive cell populations.

While compensation can be used to correct for spillover, incorrect calculation of compensation may introduce other issues into the cytometry data that negatively impact its usefulness. For example, over-compensation, under-compensation, and compensation artifacts can all contribute to inaccurate estimates of cell populations by generating data that cannot be used to distinguish between different cell populations. Examples of over and under-compensated cytometry data are shown in FIG. 10C. Over and under-compensation result in the same types of issues present in spillover. Examples of artifacts are shown in FIG. 10D. Artifacts can affect the location of cells in gating plots used to identify cell populations. These challenges are highlighted in the examples shown in FIG. 10E, FIG. 10E, FIG. 10F, FIG. 10G, and FIG. 10H. In particular, these examples illustrate the differences in high-quality and low-quality compensation.

To avoid using compensated cytometry data that may ultimately degrade the accuracy of the cytometry analysis (e.g., determining accurate cell composition percentages), the inventors have recognized that it is important to efficiently identify cytometry data with low quality compensation, and then either discard the cytometry data or adjust the compensation that has been applied to it. Accordingly, the inventors have developed machine learning techniques for efficiently determining the quality of compensation applied to cytometry data.

FIG. 11A is a flowchart of an illustrative process for determining a quality of compensation applied to flow cytometry data, according to some embodiments of the technology described herein. Process 1100 may be performed by a laptop computer, a desktop computer, one or more servers, in a cloud computing environment, computing device 108 as described herein with respect to FIG. 1A and FIG. 1D, computing device 1200 as described herein with respect to FIG. 12, or any other suitable computing device(s), as aspects of the technology described herein are not limited in this respect.

At act 1102, flow cytometry data is obtained for a biological sample. For example, the flow cytometry data may be obtained using a flow cytometry platform as part of process 1100, or it may have been previously obtained. In some embodiments, the flow cytometry data includes measurements (e.g., fluorescence intensities) for a plurality of markers. Techniques for obtaining cytometry data are described herein including at least with respect to act 202 of process 200 shown in FIG. 2A.

At act 1104, compensation is applied to the cytometry data. In some embodiments, the compensation may have been previously applied to the cytometry data. In some embodiments, applying compensation to cytometry data includes calculating the spillover signal between defined positive and negative populations (e.g., cell populations that result in a positive signal from a marker and cell populations which result in negative signal from marker). The spillover signal may then be used to determine the compensation to be applied. For example, this may include estimating a spillover matrix, in which the degree of spectral spillover between channels is estimated through single-color controls. A compensation matrix may be obtained by inverting the spillover matrix, and the compensation matrix may be applied to the cytometry data. The compensation is determined and applied using any suitable techniques, as aspects of the technology described herein are not limited in this respect. In some embodiments, software is used to perform the compensation. For example, AutoSpill is software that may be used to determine a compensation matrix. AutoSpill is described by Roca, C., et al. (“AutoSpill is a principled framework that simplifies the analysis of multichromatic flow cytometry data.” Nature communications 12.1 (2021): 2890.), which is incorporated by reference herein in its entirety.

At act 1106, a two-dimensional distribution of measurements of a pair of markers is obtained. In some embodiments, the measurements may include measurements obtained in the cytometry data at act 1102. In some embodiments, the pair of markers includes any suitable pair of the markers for which the cytometry data was obtained at act 1102. In some embodiments, the two-dimensional distribution is generated using any suitable quantile cutoff, as aspects of the technology described herein are not limited in this respect. For example, the quantile cutoff may be a value within 0.001 and 0.2, 0.002 and 0.15, 0.004 and 0.10, 0.006 and 0.08, 0.008 and, 0.05, or within any other suitable range, as aspects of the technology described herein are not limited in this respect. In some embodiments, the two-dimensional distribution is generated using any suitable number of bins, as aspects of the technology described herein are not limited in this respect. For example, the number of bins may be a value within 5 and 550, within 50 and 500, within 100 and 400, within 150 and 350, within 200 and 300, or within any other suitable range, as aspects of the technology described herein are not limited in this respect. For example, the two-dimensional distribution may be generated using a quantile cutoff of 0.01 and 256 bins.

At act 1108, pairwise correlations between the pair of markers (e.g., between the measurements obtained for the markers) are obtained. In some embodiments, the pairwise correlations are calculated using Pearsons's method, or any other suitable techniques for determining pairwise correlations, as aspects of the technology described herein are not limited in this respect. In some embodiments, a vector is used to represent at least some (e.g., all) of the pairwise correlations between the pair of marker measurements. In some embodiments, the vector represents values extracted from at least a portion of a matrix of correlation coefficients (e.g., Pearson correlation coefficients). For example, the vector may represent one or more values extracted from a triangular matrix (e.g., upper triangular matrix or lower triangular matrix). In some embodiments, the vector represents a number of features within 10 and 500, 50 and 400, 100 and 300, 150 and 200, or within any other suitable range, as aspects of the technology described herein are not limited in this respect. In some embodiments, the number of features represented by the vector is determined using Equation 11:

$$\frac{n ( n - 1 )}{2} - n = \frac{n ( n - 3 )}{2} \qquad ( Equation 11 )$$

where n is the number of markers in the cytometry data (e.g., the number of columns in the FCS file).

In some embodiments, the two-dimensional distribution and pairwise correlation features are processed using a neural network trained to predict an output indicative of the quality of compensation applied at act 1104. The neural network may include (i) a first neural network portion configured to extract features from the two-dimensional distribution, and (ii) a second neural network portion configured to predict the output indicative of the quality of compensation. Techniques for training the neural network are described herein.

At act 1110, the two-dimensional distribution is processed using the first neural network portion to extract features for the two-dimensional distribution. In some embodiments, the input to the neural network portion is a tensor image, with dimensions C×W×H (e.g., 1×256×256) representing the two-dimensional distribution. In some embodiments, the dimensions of the tensor image depend on the number of bins used to generate the two-dimensional distribution. For example, the height and width dimensions may be equal to the number of bins.

In some embodiments, the first portion of the neural network is used to extract features from the two-dimensional distribution. For example, the first portion of neural network may include one or more layers (e.g., convolutional layers) configured to extract the features from the input. For example, the first portion of the neural network may use the EfficientNet (e.g., Efficient-Net-B1) architecture. EfficientNet is described by Tan, M, and Le, Q. (“Efficientnet: Rethinking model scaling for convolutional neural networks.” International conference on machine learning. PMLR, 2019.), which is incorporated by reference herein in its entirety. An example architecture and example parameters of the first portion of the neural network are shown in FIG. 11D.

At act 1112, the extracted features and the pairwise correlations are processed using a second portion of the neural network model to obtain an output indicative of a quality of the compensation applied at act 1104. For example, the extracted features may be concatenated with a vector representative of the pairwise correlations between the marker pair, and the concatenated feature vector may be processed using the second portion of the neural network. In some embodiments, the second portion of the neural network includes a classification portion. For example, the second portion of the neural network includes a single-layer or multi-layer perceptron.

In some embodiments, given the concatenated feature vector, the second portion of the neural network outputs a quality associated with the compensation applied to the cytometry data. For example, the neural network may be trained to predict whether the quality is greater than or equal to a threshold quality. Additionally, or alternatively, the neural network may be trained to predict a label (e.g., whether the quality of compensation is high, low, and/or can be adjusted).

At act 1114, process 1100 includes determining whether to discard the cytometry data based on the output of the second neural network model. In some embodiments, process 1100 includes determining to discard the cytometry data if the output of the neural network indicates that the quality is lower than a threshold quality and/or outputs a label indicating that the cytometry data is low quality and/or should be discarded. In some embodiments, when it is determined that the cytometry data is to be discarded, a recommendation may be output, at act 1116, indicating same. Additionally, or alternatively, the cytometry data may be discarded.

At act 1118, process 1100 includes determining whether to adjust one or more aspects of the compensation applied to the cytometry data based on the output of the second neural network model. In some embodiments, process 1100 includes determining to adjust the cytometry data if the output of the neural network indicates that the quality is between an upper and lower bound and/or outputs a label indicating that the cytometry data can or should be adjusted. In some embodiments, when it is determined that the cytometry data is to be adjusted, a recommendation may be output, at act 1120, indicating same. Additionally, or alternatively, the cytometry data may be adjusted.

At act 1122, process 1100 includes determining whether there is another pair of markers to be analyzed for the purpose of determining the quality of the compensation applied to the cytometry data, according to some embodiments of the technology described herein. When there is another pair of markers, at least some (e.g., all) of acts 1106-1118 may be repeated for the next pair. In some embodiments, process 1100 may be repeated for at least some (e.g., all) of the marker pairs in the cytometry data.

FIG. 11B shows two examples of two-dimensional distributions of marker pairs labeled as having a compensation quality that is greater than or equal to a threshold quality, according to some embodiments of the technology described herein. Both examples show the two-dimensional distribution of the marker pair: CD66b and CD193 CCR3. These may be labeled as “high quality.”

FIG. 11C shows two examples of two-dimensional distributions of marker pairs labeled as having a compensation quality that is less than or equal to the threshold quality, according to some embodiments of the technology described herein. The two examples show the two-dimensional distributions of marker pair CD66b and CD56 and marker pair: CD56 and CD123.

Compared to the examples shown in FIG. 11B, the examples shown in FIG. 11C include “diagonal features,” indicating that the marker values may have a correlation resulting from the inaccurate application of compensation. These may be labeled as “low quality.” FIG. 11D is an example of an architecture and parameters of a neural network used to determine a quality of compensation applied to cytometry data, according to some embodiments of the technology described herein. As shown, an input representative of a two-dimensional distribution of marker pairs 1105 is processed using a first portion 1115 of the neural network to extract features 1150 for the two-dimensional distribution. The extracted features 1150 and the features representative of the pairwise correlations 1140 between the marker pairs are processed using the second portion 1160 of the neural network to obtain an output indicative of the quality of compensation applied to the cytometry data.

As described with respect to FIG. 11A, a neural network may be trained to predict a quality of compensation applied to cytometry data, given (i) input representing a two-dimensional distribution of marker pairs and (ii) input representing pairwise correlation between the marker pairs. The neural network classifier may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used. Any suitable loss function may be used such as, for example, a binary cross-entropy loss function.

In some embodiments, the neural network model is trained using training data, each training sample including (i) a two-dimensional distribution of marker pairs, (ii) pairwise correlations between the marker pairs, and (iii) a corresponding label indicating the quality of the compensation applied to the cytometry data used to generate the distribution and pairwise correlations. For example, a training sample may be labeled as “high quality” or “low quality.” Additionally, or alternatively, the labels may include a label indicating that the compensation can and/or should be adjusted (e.g., “fixlate”). In some embodiments, this label may be assigned using a soft labeling technique. In some embodiments, a soft labeling approach is used to integrate the “fixlate” category into the binary classification task. For example, the approach may involve assigning a weight to the “fixlate” class.

In some embodiments, to minimize overfitting, one or more augmentation techniques may be used to augment the training data. For example, the markers may be rearranged. Additionally, or alternatively, distribution modulation may be performed (e.g., random Gamma augmentation). Additionally, or alternatively, noise elements may be added to the training data (e.g., positive Gaussian noise, Gaussian blur, and/or random crop). Hyperparameter tuning may be used to select the strength and quantity of these augmentations.

In some embodiments, one or more techniques may be performed to enhance convergence and stabilize the training process. For example, during hyperparameter optimization, the learning rate scheduler may gradually reduce the learning rate. Additionally, or alternatively, the learning rate may incrementally increase from an initial value to a target value in the first n epochs.

### Machine Learning

In some embodiments, the machine learning model may include a decision tree classifier, a gradient boosted decision tree classifier, a neural network, a support vector machine classifier, or any other suitable type of machine learning model, as aspects of the technology described herein are not limited in this respect. In some embodiments, the machine learning model may include an ensemble of machine learning models of any suitable type (the machine learning models part of the ensemble may be termed “weak learners”). For example, the machine learning model may include an ensemble of decision tree classifiers.

As described above, in some embodiments, the machine learning model may be implemented as a decision tree classifier. Any suitable type of decision tree classifier may be used and may be trained using any suitable supervised decision tree learning technique. For example, the decision tree classifier may be trained by the iterative dichotomiser technique (e.g., the ID3 algorithm as described, for example, in Quinlan, J. R. 1986. Induction of Decision Trees. Mach. Learn. 1, 1 (March 1986), 81-106)), the C4.5 technique (e.g., as described, for example, in Quinlan, J. R. C4.5: Programs for Machine Learning. Morgan Kaufmann Publishers, 1993), the classification and regression tree (CART) technique (e.g., as described, for example, in Breiman, Leo; Friedman, J. H.; Olshen, R. A.; Stone, C. J. (1984). Classification and regression trees. Monterey, CA: Wadsworth & Brooks/Cole Advanced Books & Software). It should be appreciated that a decision tree classifier may be trained using any other suitable training method, as aspects of the technology described herein are not limited in this respect.

In some embodiments, a gradient-boosted decision tree classifier may be used. The gradient-boosted decision tree classifier may be an ensemble of multiple decision tree classifiers (sometimes called “weak learners”). The prediction (e.g., classification) generated by the gradient-boosted decision tree classifier is formed based on the predictions generated by the multiple decision trees part of the ensemble. The ensemble may be trained using an iterative optimization technique involving calculation of gradients of a loss function (hence the name “gradient” boosting). Any suitable supervised training algorithm may be applied to training a gradient-boosted decision tree classifier including, for example, any of the algorithms described in Hastie, T.; Tibshirani, R.; Friedman, J. H. (2009). “10. Boosting and Additive Trees”. The Elements of Statistical Learning (2nd ed.). New York: Springer. pp. 337-384. In some embodiments, the gradient-boosted decision tree classifier may be implemented using any suitable publicly-available gradient boosting framework such as XGBoost (e.g., as described, for example, in Chen, T., & Guestrin, C. (2016). XGBoost: A Scalable Tree Boosting System. In Proceedings of the 22nd ACM SIGKDD International Conference on Knowledge Discovery and Data Mining (pp. 785-794). New York, NY, USA: ACM.). The XGBoost software may be obtained from http://xgboost.ai, for example). Another example framework that may be employed is LightGBM (e.g., as described, for example, in Ke, G., Meng, Q., Finley, T., Wang, T., Chen, W., Ma, W., . . . Liu, T.-Y. (2017). Lightgbm: A highly efficient gradient boosting decision tree. Advances in Neural Information Processing Systems, 30, 3146-3154.). The LightGBM software may be obtained from https://lightgbm.readthedocs.io/, for example).

In some embodiments, a neural network classifier may be used. The neural network classifier may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used.

### Computer Implementation

An illustrative implementation of a computer system 1200 that may be used in connection with any of the embodiments of the technology described herein (e.g., such as the method of FIGS. 2A-B and 4A-C) is shown in FIG. 12. The computer system 1200 includes one or more processors 1210 and one or more articles of manufacture that comprise non-transitory computer-readable storage media (e.g., memory 1220 and one or more non-volatile storage media 1230). The processor 1210 may control writing data to and reading data from the memory 1220 and the non-volatile storage device 1230 in any suitable manner, as the aspects of the technology described herein are not limited to any particular techniques for writing or reading data. To perform any of the functionality described herein, the processor 1210 may execute one or more processor-executable instructions stored in one or more non-transitory computer-readable storage media (e.g., the memory 1220), which may serve as non-transitory computer-readable storage media storing processor-executable instructions for execution by the processor 1210.

Computing device 1200 may also include a network input/output (I/O) interface 1240 via which the computing device may communicate with other computing devices (e.g., over a network), and may also include one or more user I/O interfaces 1250, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel. It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

### Biological Samples

Any of the methods, systems, or other claimed elements may use or be used to analyze a biological sample from a subject. In some embodiments, a biological sample is obtained from a subject having, suspected of having cancer, or at risk of having cancer. The biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, or skin tissue), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of a tissue, in some embodiments, refers to a sample comprising cells from a tissue. In some embodiments, the sample of the tumor comprises non-cancerous cells from a tissue. In some embodiments, the sample of the tumor comprises precancerous cells from a tissue.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue, or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

Any of the biological samples described herein may be obtained from the subject using any known technique. See, for example, the following publications on collecting, processing, and storing biological samples, each of which are incorporated by reference herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011; (163):23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (i.e., a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixatuve. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris-Cl; 0.5 mM EDTA, pH 9.0)) and other coagulants, and Acids Citrate Dextronse (e.g., for blood specimens). In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −80° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

Methods of the present disclosure encompass obtaining one or more biological samples from a subject for analysis. In some embodiments, one biological sample is collected from a subject for analysis. In some embodiments, more than one (e.g., 2, 3, 4, 5, 6, 7, 8, 9, 10, 11, 12, 13, 14, 15, 16, 17, 18, 19, 20, or more) biological samples are collected from a subject for analysis. In some embodiments, one biological sample from a subject will be analyzed. In some embodiments, more than one (e.g., 2, 3, 4, 5, 6, 7, 8, 9, 10, 11, 12, 13, 14, 15, 16, 17, 18, 19, 20, or more) biological samples may be analyzed. If more than one biological sample from a subject is analyzed, the biological samples may be procured at the same time (e.g., more than one biological sample may be taken in the same procedure), or the biological samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure).

A second or subsequent biological sample may be taken or obtained from the same region (e.g., from the same tumor or area of tissue) or a different region (including, e.g., a different tumor). A second or subsequent biological sample may be taken or obtained from the subject after one or more treatments and may be taken from the same region or a different region. As a non-limiting example, the second or subsequent biological sample may be useful in determining whether the cancer in each biological sample has different characteristics (e.g., in the case of biological samples taken from two physically separate tumors in a patient) or whether the cancer has responded to one or more treatments (e.g., in the case of two or more biological samples from the same tumor or different tumors prior to and subsequent to a treatment). In some embodiments, each of the at least one biological sample is a bodily fluid sample, a cell sample, or a tissue biopsy sample.

In some embodiments, one or more biological specimens are combined (e.g., placed in the same container for preservation) before further processing. For example, a first sample of a first tumor obtained from a subject may be combined with a second sample of a second tumor from the subject, wherein the first and second tumors may or may not be the same tumor. In some embodiments, a first tumor and a second tumor are similar but not the same (e.g., two tumors in the brain of a subject). In some embodiments, a first biological sample and a second biological sample from a subject are sample of different types of tumors (e.g., a tumor in muscle tissue and brain tissue).

### Subjects

Aspects of this disclosure relate to a biological sample that has been obtained from a subject. In some embodiments, a subject is a mammal (e.g., a human, a mouse, a cat, a dog, a horse, a hamster, a cow, a pig, or other domesticated animal). In some embodiments, a subject is a human. In some embodiments, a subject is an adult human (e.g., of 18 years of age or older). In some embodiments, a subject is a child (e.g., less than 18 years of age). In some embodiments, a human subject is one who has or has been diagnosed with at least one form of cancer.

In some embodiments, a cancer from which a subject suffers is a carcinoma, a sarcoma, a myeloma, a leukemia, a lymphoma, or a mixed type of cancer that comprises more than one of a carcinoma, a sarcoma, a myeloma, a leukemia, and a lymphoma. Carcinoma refers to a malignant neoplasm of epithelial origin or cancer of the internal or external lining of the body. Sarcoma refers to cancer that originates in supportive and connective tissues such as bones, tendons, cartilage, muscle, and fat. Myeloma is cancer that originates in the plasma cells of bone marrow. Leukemias (“liquid cancers” or “blood cancers”) are cancers of the bone marrow (the site of blood cell production). Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Non-limiting examples of a mixed type of cancer include adenosquamous carcinoma, mixed mesodermal tumor, carcinosarcoma, and teratocarcinoma. In some embodiments, a subject has a tumor. A tumor may be benign or malignant. In some embodiments, a cancer is any one of the following: skin cancer, lung cancer, breast cancer, prostate cancer, colon cancer, rectal cancer, cervical cancer, and cancer of the uterus.

In some embodiments, a subject is at risk for developing cancer, e.g., because the subject has one or more genetic risk factors, or has been exposed to or is being exposed to one or more carcinogens (e.g., cigarette smoke, or chewing tobacco).

### Flow Cytometry

In some embodiments, a flow cytometry platform may be used to perform flow cytometry investigation of a fluid sample. The fluid sample may include target particles with particular particle attributes. The flow cytometry investigation of the fluid sample may provide a flow cytometry result for the fluid sample.

In some embodiments, the fluid sample may be exposed to a stain or dye that provides response radiation when exposed to investigation excitation radiation that may be measured by the radiation detection system of the flow cytometry platform. In some embodiments, a multiplicity of photodetectors is included in the flow cytometry platform. When a particle passes through the laser beam, time correlated pulses on forward scatter (FSC) and side scatter (SSC) detectors, and possibly also fluorescent emission detectors will occur. This is an “event,” and for each event the magnitude of the detector output for each detector, FSC, SSC and fluorescence detectors is stored. The data obtained comprise the signals measured for each of the light scatter parameters and the fluorescence emissions.

Flow cytometry platforms may further comprise components for storing the detector outputs and analyzing the data. For example, data storage and analysis may be carried out using a computer connected to the detection electronics. For example, the data can be stored logically in tabular form, where each row corresponds to data for one particle (or one event), and the columns correspond to each of the measured parameters. The use of standard file formats, such as an “FCS” file format, for storing data from a flow cytometer facilitates analyzing data using separate programs and/or machines. In some embodiments, the data may be displayed in 2-dimensional (2D) plots for ease of visualization, but other methods may be used to visualize multidimensional data.

In some embodiments, the parameters measured using a flow cytometer may include FSC, which refers to the excitation light that is scattered by the particle along a generally forward direction, SSC, which refers to the excitation light that is scattered by the particle in a generally sideways direction, and the light emitted from fluorescent molecules in one or more channels (frequency bands) of the spectrum, referred to as FL1, FL2, etc., or by the name of the fluorescent dye that emits primarily in that channel.

Both flow and scanning cytometers are commercially available from, for example, BD Biosciences (San Jose, Calif.). Flow cytometry is described in, for example, Landy et al. (eds.), Clinical Flow Cytometry, Annals of the New York Academy of Sciences Volume 677 (1993); Bauer et al. (eds.), Clinical Flow Cytometry: Principles and Applications, Williams & Wilkins (1993); Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Practical Shapiro, Flow Cytometry, 4th ed., Wiley-Liss (2003); all incorporated herein by reference. Fluorescence imaging microscopy is described in, for example, Pawley (ed.), Handbook of Biological Confocal Microscopy, 2nd Edition, Plenum Press (1989), incorporated herein by reference

### Mass Cytometry

In some embodiments, a mass cytometry platform may be used to perform mass cytometry investigation of a fluid sample. The fluid sample may include target particles with particular particle attributes. The mass cytometry investigation of the fluid sample may provide a mass cytometry result for the fluid sample.

In some embodiments, the fluid sample may be exposed to target-specific antibodies labeled with metal isotopes. In some embodiments, elemental mass spectrometry (e.g., inductively coupled plasma mass spectrometry (ICP-MS) and time of flight mass spectrometry (TOF-MS)) is used to detect the conjugated antibodies. For example, elemental mass spectrometry can discriminate isotopes of different atomic weights and measure electrical signals for isotopes associated with each particle or cell. Data obtained for a single cell or particle is considered an “event.”

Mass cytometry platforms may further comprise components for storing the detector outputs and analyzing the data. For example, data storage and analysis may be carried out using a computer connected to the detection elements. The use of standard file formats, such as an “FCS” file format, for storing data from a mass cytometry platform facilitates analyzing data using separate programs and/or machines.

Mass cytometry platforms are commercially available from, for example, Fluidigm (San Francisco, CA). Mass cytometry is described in, for example, Bendall et al., A deep profiler's guide to cytometry, Trends in Immunology, 33(7), 323-332 (2012) and Spitzer et al., Mass Cytometry: Single Cells, Many Features, Cell, 165(4), 780-791 (2016), both of which are incorporated by reference herein in their entirety.

### Methods of Treatment

In certain methods described herein, an effective amount of anti-cancer therapy described herein may be administered or recommended for administration to a subject (e.g., a human) in need of the treatment via a suitable route (e.g., intravenous administration).

The subject to be treated by the methods described herein may be a human patient having, suspected of having, or at risk for a cancer. Examples of a cancer are provided herein. At the time of diagnosis, the cancer may be cancer of unknown primary. The subject to be treated by the methods described herein may be a mammal (e.g., may be a human). The subject to be treated by the methods described herein may be a mammal (e.g., may be a human).

A subject having a cancer may be identified by routine medical examination, e.g., laboratory tests, biopsy, PET scans, CT scans, or ultrasounds. A subject suspected of having a cancer might show one or more symptoms of the disorder, e.g., unexplained weight loss, fever, fatigue, cough, pain, skin changes, unusual bleeding or discharge, and/or thickening or lumps in parts of the body. A subject at risk for a cancer may be a subject having one or more of the risk factors for that disorder. For example, risk factors associated with cancer include, but are not limited to, (a) viral infection (e.g., herpes virus infection), (b) age, (c) family history, (d) heavy alcohol consumption, (e) obesity, and (f) tobacco use.

The dosage of anti-cancer therapy administered to a subject may vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor formation, tumor growth, molecular category identified for the cancer using the techniques described herein) may be analyzed.

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward a cancer.

Alleviating a cancer includes delaying the development or progression of the disease or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given period and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

Conventional methods, known to those of ordinary skill in the art of medicine, may be used to administer the anti-cancer therapeutic agent to the subject, depending upon the type of disease to be treated or the site of the disease. The anti-cancer therapeutic agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, an anti-cancer therapeutic agent may be administered to the subject via injectable depot routes of administration such as using 1-, 3-, or 6-month depot injectable or biodegradable materials and methods.

In one embodiment, an anti-cancer therapeutic agent is administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568, the contents of each of which are incorporated by reference herein for this purpose.

In some embodiments, more than one anti-cancer therapeutic agent, such as an antibody and a small molecule inhibitory compound, may be administered to a subject in need of the treatment. The agents may be of the same type or different types from each other. At least one, at least two, at least three, at least four, or at least five different agents may be co-administered. Generally anti-cancer agents for administration have complementary activities that do not adversely affect each other. Anti-cancer therapeutic agents may also be used in conjunction with other agents that serve to enhance and/or complement the effectiveness of the agents.

Treatment efficacy can be assessed by methods well-known in the art, e.g., monitoring tumor growth or formation in a patient subjected to the treatment. Alternatively, or in addition to, treatment efficacy can be assessed by monitoring tumor type over the course of treatment (e.g., before, during, and after treatment).

In some embodiments, an anti-cancer therapeutic agent is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor (e.g., nivolumab (Opdivo), pembrolizumab (Keytruda), atezolizumab (Tecentriq), avelumab (Bavencio), durvalumab (Imfinzi)), a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for identifying types of cells present in a biological sample using flow cytometry performed using a panel of markers and a set of machine learning models each of which corresponds to a respective marker in the panel of markers, the method comprising:
using at least one computer hardware processor to perform:
obtaining flow cytometry data for the biological sample, the biological sample previously obtained from a subject and comprising a plurality of cells including a first cell, the flow cytometry data including flow cytometry measurements of cells in the plurality of cells that were obtained by a flow cytometry platform; and
identifying cell types of at least a subset of the plurality of cells using the set of machine learning models to obtain a plurality of cell types, the set of machine learning models including a first machine learning model corresponding to a first marker in the panel of markers and a second machine learning model corresponding to a second marker in the panel of markers, the identifying comprising:
obtaining, from the flow cytometry data, first flow cytometry measurements of the first cell obtained by the flow cytometry platform;
processing the first flow cytometry measurements using the first machine learning model to obtain first output indicating a degree to which the first marker is expressed in the first cell;
processing the first flow cytometry measurements using the second machine learning model to obtain second output indicating a degree to which the second marker is expressed in the first cell; and
identifying a cell type for the first cell using the first output indicating the degree to which the first marker is expressed in the first cell and the second output indicating the degree to which the second marker is expressed in the first cell.

2. The method of claim 1, wherein the subset of the plurality of cells comprises at least 100 cells, at least 500 cells, at least 1,000 cells, at least 2,500 cells, at least 5,000 cells, at least 10,000 cells, at least 25,000 cells, at least 75,000 cells, at least 100,000 cells, at least 150,000 cells, at least 200,000 cells, or at least 500,000 cells.

3. The method of claim 1, wherein identifying cell types of at least the subset of the plurality of cells using the set of machine learning models further comprises, for a second cell in the plurality of cells,
obtaining, from the flow cytometry data, second flow cytometry measurements of the second cell obtained by the flow cytometry platform;
processing the second flow cytometry measurements using the first machine learning model to obtain third output indicating a degree to which the first marker is expressed in the second cell;
processing the second flow cytometry measurements using the second machine learning model to obtain fourth output indicating a degree to which the second marker is expressed in the second cell; and
identifying a cell type for the second cell using the third output indicating the degree to which the first marker is expressed in the second cell and the fourth output indicating the degree to which the second marker is expressed in the second cell.

4. The method of claim 1, wherein the first machine learning model is a binary classifier.

5. The method of claim 1, wherein the first machine learning model is a decision tree classifier, a gradient-boosted decision tree classifier, or a neural network.

6. The method of claim 5, wherein the first machine learning model comprises a gradient-boosted decision tree classifier, and the gradient-boosted decision tree classifier comprises an ensemble of decision tree classifiers.

7. The method of claim 1, further comprising:
processing the first flow cytometry measurements using each of the set of machine learning models to obtain multiple outputs each indicating a degree to which a respective marker in the panel of markers is expressed in the first cell, the multiple outputs including the first output and the second output; and
identifying the cell type for the first cell using the multiple outputs.

8. The method of claim 1, wherein identifying the cell type for the first cell using the first output and the second output comprises identifying the cell type from the group of cell types listed in Table 1B, Table 2C, Table 7B, Table 8C, or Table 10C.

9. The method of claim 1,
wherein the panel of markers comprises: CD45, CD66b, and CD193 CCR3,
wherein the set of machine learning models comprises:
a machine learning model trained to predict, from flow cytometry measurements of a cell, whether CD45 is expressed by the cell, the flow cytometry measurements of the cell including expression levels for a plurality of markers including CD45, CD66b, and CD193 CCR3,
a machine learning model trained to predict, from the flow cytometry measurements of the cell, whether CD66b is expressed by the cell, and
a machine learning model trained to predict, from the flow cytometry measurements of the cell, whether CD193 CCR3 is expressed by the cell.

10. The method of claim 9, wherein identifying cell types of at least the subset of the plurality of cells using the set of machine learning models further comprises, for a second cell in the plurality of cells:
obtaining, from the flow cytometry data, second flow cytometry measurements of the second cell obtained by the flow cytometry platform;
processing the second flow cytometry measurements using the machine learning model trained to predict whether CD45 is expressed by the cell to obtain output indicating whether CD45 is expressed in the second cell;
processing the second flow cytometry measurements using the machine learning model trained to predict whether CD66b is expressed by the cell to obtain output indicating whether CD66b is expressed in the second cell;
processing the second flow cytometry measurements using the machine learning model trained to predict whether CD193 CCR3 is expressed by the cell to obtain output indicating whether CD193 CCR3 is expressed in the second cell; and
identifying a type for the second cell using the output indicating whether CD45 is expressed in the second cell, the output indicating whether CD66b is expressed in the second cell, and the output indicating whether CD193 CCR3 is expressed in the second cell.

11. The method of claim 10, wherein identifying the type for the second cell comprises:
identifying eosinophil as the type for the second cell when CD45, CD66b, and CD193 CCR3 are each expressed in the second cell;
identifying neutrophil as the type for the second cell when CD45 and CD66b are expressed in the second cell, and when CD193 CCR3 is not expressed in the second cell; and
identifying basophil as the type for the second cell when CD45 and CD193 CCR3 are expressed in the second cell, and when CD66b is not expressed in the second cell.

12. The method of claim 1, further comprising:
determining cell composition percentages of different types of cells in the biological sample based on the identified cell types.

13. The method of claim 12, wherein the subject has, is suspected of having, or is at risk of having cancer, and wherein the method further comprises:
identifying a treatment for the subject based on the determined cell composition percentages.

14. The method of claim 13, further comprising administering the identified treatment to the subject.

15. The method of claim 13, wherein identifying the treatment for subject based on the determined cell composition percentages comprises:
identifying an antibody anti-cancer agent for the subject when a cell composition percentage of peripheral blood mononuclear cells (PBMCs) is below a threshold.

16. The method of claim 13, wherein identifying the treatment for the subject based on the determined cell composition percentages comprises:
determining a ratio between a cell composition percentage of CD8+PD-1+ cells and a cell composition percentage of CD4+PD-1; and
identifying immune checkpoint blockade therapy for the subject when the determined ratio is above a threshold.

17. The method of claim 1, further comprising:
identifying a first plurality of cell types present in a first subsample of the biological sample using flow cytometry performed using a first panel of markers and a first set of machine learning models, the first plurality of cell types including a first cell type;
identifying a second plurality of cell types present in a second subsample of the biological sample using flow cytometry performed using a second panel of markers and a second set of machine learning models, the second plurality of cell types including the first cell type;
determining, for each particular cell type of at least some of the cell types included in the first plurality of cell types and the second plurality of cell types, a cell composition percentage for the particular cell type, the determining comprising:
determining a first number of cells of the particular cell type in the first plurality of cell types;
determining a second number of cells of the particular cell type in the second plurality of cell types;
normalizing the second number of cells of the particular cell type with respect to a number of cells of the first cell type included in the first plurality of cell types and a number of cells of the first cell type included in the second plurality of cell types; and
determining the cell composition percentage for the particular cell type based on the first number of cells of the particular cell type and the normalized second number of cells of the particular cell type.

18. The method of claim 1, wherein a compensation matrix was applied to the flow cytometry data, and wherein the method further comprises:
processing at least a portion of the cytometry data using a trained neural network model to obtain an output indicative of a quality of the compensation matrix; and
determining, based on the output indicative of the quality of the compensation matrix, whether to discard the cytometry data.

19. At least one non-transitory computer-readable storage medium storing processor-executable instructions that, when executed by at least one computer hardware processor, cause the at least one computer hardware processor to perform a method for identifying types of cells present in a biological sample using flow cytometry performed using a panel of markers and a set of machine learning models each of which corresponds to a respective marker in the panel of markers, the method comprising:
obtaining flow cytometry data for the biological sample, the biological sample previously obtained from a subject and comprising a plurality of cells including a first cell, the flow cytometry data including flow cytometry measurements of cells in the plurality of cells that were obtained by a flow cytometry platform; and
identifying cell types of at least a subset of the plurality of cells using the set of machine learning models to obtain a plurality of cell types, the set of machine learning models including a first machine learning model corresponding to a first marker in the panel of markers and a second machine learning model corresponding to a second marker in the panel of markers, the identifying comprising:
obtaining, from the flow cytometry data, first flow cytometry measurements of the first cell obtained by the flow cytometry platform;
processing the first flow cytometry measurements using the first machine learning model to obtain first output indicating a degree to which the first marker is expressed in the first cell;
processing the first flow cytometry measurements using the second machine learning model to obtain second output indicating a degree to which the second marker is expressed in the first cell; and
identifying a cell type for the first cell using the first output indicating the degree to which the first marker is expressed in the first cell and the second output indicating the degree to which the second marker is expressed in the first cell.

20. A system comprising:
at least one computer hardware processor; and
at least one non-transitory computer-readable storage medium storing processor-executable instructions that, when executed by the at least one computer hardware processor, cause the at least one computer hardware processor to perform a method for identifying types of cells present in a biological sample using flow cytometry performed using a panel of markers and a set of machine learning models each of which corresponds to a respective marker in the panel of markers, the method comprising:
obtaining flow cytometry data for the biological sample, the biological sample previously obtained from a subject and comprising a plurality of cells including a first cell, the flow cytometry data including flow cytometry measurements of cells in the plurality of cells that were obtained by a flow cytometry platform; and
identifying cell types of at least a subset of the plurality of cells using the set of machine learning models to obtain a plurality of cell types, the set of machine learning models including a first machine learning model corresponding to a first marker in the panel of markers and a second machine learning model corresponding to a second marker in the panel of markers, the identifying comprising:
obtaining, from the flow cytometry data, first flow cytometry measurements of the first cell obtained by the flow cytometry platform;
processing the first flow cytometry measurements using the first machine learning model to obtain first output indicating a degree to which the first marker is expressed in the first cell;
processing the first flow cytometry measurements using the second machine learning model to obtain second output indicating a degree to which the second marker is expressed in the first cell; and
identifying a cell type for the first cell using the first output indicating the degree to which the first marker is expressed in the first cell and the second output indicating the degree to which the second marker is expressed in the first cell.
